(19) United States
(12) Patent Application Publication
(10) Pub. No.: US 2018/0258408 A1
(43) Pub. Date: Sep. 13, 2018

(54) MODIFIED LYSOSOMAL PROTEIN AND PRODUCTION THEREOF

(71) Applicant: Swedish Orphan Biovitrum AB (publ), Stockholm (SE)

(72) Inventors: Erik Nordling, Dandling (SE); Patrick Strömberg, Sollentuna (SE); Stefan Svensson Gelius, Älvsjö (SE)

(21) Appl. No.: 15/764,175

(22) PCT Filed: Sep. 30, 2016
(86) PCT No.: PCT/EP2016/073452

§ 371 Date: Mar. 28, 2018

(30) Foreign Application Priority Data
Oct. 1, 2015 (EP) 15187877.4

(51) Int. Cl. C12N 9/14; C12N 9/16; C12N 9/24

(52) U.S. Cl. CPC C12N 9/14; C12N 9/16; C12Y 301/06013; A61K 38/00; C12Y 302/01076; C12Y 310/01001; C12N 9/2402

(57) ABSTRACT

Disclosed herein are a modified lysosomal protein, methods for preparing a modified lysosomal protein and therapeutic use of such a modified protein. Further disclosed herein is a method of treating a mammal afflicted with a lysosomal storage disease. In particular, the present disclosure relates to a method of preparing a modified lysosomal protein, said method comprising reacting a glycosylated lysosomal protein with an alkali metal periodate and reacting said lysosomal protein with an alkali metal borohydride for a time period of no more than 2 h, thereby modifying glycan moieties of the lysosomal protein and reducing the activity of the lysosomal protein with respect to glycan recognition receptors.

### TECHNICAL FIELD

The present disclosure relates to a modified lysosomal protein, compositions comprising a modified lysosomal protein and methods for producing a modified lysosomal protein. Furthermore, use of a modified lysosomal protein in therapy such as in treatment of a lysosomal storage disease is disclosed.

### BACKGROUND

Lysosomal Storage Disease

The lysosomal compartment functions as a catabolic machinery that degrades waste material in cells. Degradation is achieved by a number of hydrolases and transporters compartmentalized specifically to the lysosome. There are today over 40 identified inherited diseases where a link has been established between disease and mutations in genes coding for lysosomal proteins. These diseases are defined as lysosomal storage diseases (LSDs) and are characterized by a buildup of a metabolite (or metabolites) that cannot be degraded due to the insufficient degrading capacity. As a consequence of the excess lysosomal storage of the metabolite, lysosomes increase in size. How the accumulated storage material causes pathology is not fully understood but may involve mechanisms such as inhibition of autophagy and induction of cell apoptosis (Cox & Cachón-Gonzalez, J Pathol 226: 241-254 (2012)).

Enzyme/Protein Replacement Therapy

The missing function caused by a mutated or missing protein may be restored by administration and thus replacement of the mutated/missing protein with a protein from a heterologous source. This has been shown for a variety of disease fields. Within the field of hemophilia, administration of both enzymes, such as factor IX and factor VII, and proteins, such as factor VIII, that are part of activation complexes in the coagulation pathway have been successfully employed. These components are of course present in the blood and thus it is easy to administrate a protein to its site of action.

In the field of lysosomal storage diseases, storage can be reduced by administration of a lysosomal enzyme from a heterologous source. It is well established that intravenous administration of a lysosomal enzyme results in its rapid uptake by cells via a mechanism called receptor mediated endocytosis. This endocytosis is mediated by receptors on the cell surface, and in particular the two mannose-6 phosphate receptors (M6PR) have been shown to be pivotal for uptake of certain lysosomal enzymes (Neufeld; Birth Defects Orig Artic Ser 16: 77-84 (1980)). M6PR recognize phosphorylated oligomannose glycans which are characteristic for lysosomal proteins.

Based on the principle of receptor mediated endocytosis, enzyme replacement therapies (ERT) are today available for seven LSDs, (Gaucher, Fabrys, Pompe and the Mucopolysaccharidosis type I, II, IVA and VI). These therapies are efficacious in reducing lysosomal storage in various peripheral organs and thereby ameliorate some symptoms related to the pathology. Elaprase® and Aldurazyme® are examples of orphan medicinal products indicated for long-term treatment of patients with Hunter syndrome (Mucopolysaccharidosis II, MPSII) and the non-neurological symptoms of patients with Hurler/Scheie syndrome (Mucopolysaccharidosis I, MPS I). Both enzymes essentially function to reduce lysosomal storage by hydrolysis of glycosaminoglycans (GAGs) dermatan sulfate and heparan sulfate. Reduced or absent activity of any of these enzymes results in an intracellular accumulation of these GAGs, which causes a progressive and clinically heterogeneous disorder with multiple organ and tissue involvement.

A majority of the LSDs however causes build-up of lysosomal storage in the central nervous system (CNS) and consequently presents a repertoire of CNS related signs and symptoms. A major drawback with intravenously administered ERT is the poor distribution to the CNS. The CNS is protected from exposure to blood borne compounds by the blood brain barrier (BBB), formed by the CNS endothelium. The endothelial cells of the BBB exhibit tight junctions which prevent paracellular passage, show limited passive endocytosis and in addition lack some of the receptor mediated transcytotic capacity seen in other tissues. Notably, in mice M6PR mediated transport across the BBB is only observed up to two weeks after birth (Urayama et al, Mol Ther 16: 1261-1266 (2008)).

In addition to the neurological component of LSDs, peripheral pathology is to some extent also sub-optimally addressed in current enzyme replacement treatment. Patients frequently suffer from arthropathy, clinically manifested in joint pain and stiffness resulting in severe restriction of motion. Moreover, progressive changes in the thoracic skeleton may cause respiratory restriction.

Prevailing storage leading to thickening of the heart valves along with the walls of the heart can moreover result in progressive decline in cardiac function. Also pulmonary function can further regress despite enzyme replacement treatment.

Glycosylation of Lysomal Enzymes

In general, N-glycosylations can occur at an Asn-X-Ser/Thr sequence motif. To this motif the initial core structure of the N-glycan is transferred by the glycosyltransferase oligosaccharyltransferase, within the reticular lumen. This common basis for all N-linked glycans is made up of 14 residues; 3 glucose, 9 mannose, and 2 N-acetylglucosamine. This precursor is then converted into three general types of N-glycans; oligomannose, complex and hybrid (FIG. 7), by the actions of a multitude of enzymes that both trims down the initial core and adds new sugar moieties. Each mature N-glycan contains the common core Man(Man)2-GlcNAc-GlcNAc-Asn, where Asn represents the attachment point to the protein. In yeast, oligomannose glycans can be extended to contain up to 200 mannose moieties in a repetive fashion depicted at the far right in FIG. 7 (Dean, Biochimica et Biophysica Acta 1426:309-322 (1999)).

In addition, proteins directed to the lysosome carry one or more N-glycans which are phosphorylated. The phosphorylation occurs in the Golgi and is initiated by the addition of N-acetylglucosamine-1-phosphate to C-6 of mannose residues of oligomannose type N-glycans. The N-acetylglucosamine is cleaved off to generate Mannose-6-phospate (M6P) residues, that are recognized by M6PRs and will initiate the transport of the lysosomal protein to the lysosome. The resulting N-glycan is then trimmed to the point where the M6P is the terminal group of the N-glycan chain. (Essentials of Glycobiology. 2nd edition. Varki A, Cummings R D, Esko J D, et al, editors. Cold Spring Harbor (N.Y.): Cold Spring Harbor Laboratory Press; 2009.)

The binding site of the M6PR requires a terminal M6P group that is complete, as both the sugar moiety and the phosphate group is involved in the binding to the receptor (Kim et al, Curr Opin Struct Biol 19(5):534-42 (2009)).

Enzyme Replacement Therapy Targeting the Brain by Glycan Modification

A potential strategy to increase distribution of lysosomal enzyme to the CNS has been disclosed in WO 2008/109677. In this published application, chemical modification of β-glucuronidase using sodium meta-periodate and sodium borohydride is described (see also Grubb et al, Proc Natl Acad Sci USA 105: 2616-2621 (2008)). This modification, consisting of oxidation with 20 mM sodium periodate for 6.5 h, followed by quenching, dialysis and reduction with 100 mM sodium borohydride overnight (referred to hereinafter as known method), substantially improved CNS distribution of β-glucuronidase and resulted in clearance of neuronal storage in a murine model of the LSD mucopolysaccharidosis VII. Although the underlying mechanism of brain distribution is unclear, it was noted that the chemical modification disrupted glycan structure on β-glucuronidase and it was further demonstrated that receptor mediated endocytosis by M6PR was strongly reduced.

The chemical modification strategy has been investigated for other lysosomal enzymes. For example, modification according to the known method did not improve distribution to the brain of intravenously administrated protease tripeptidyl peptidase I (Meng et al, PLoS One (2012)). Neither has satisfactory results been demonstrated for sulfamidase. Sulfamidase, chemically modified according to the known method, did indeed display an increased half-life in mice but no effect in the brain of MPS-IIIIA mice. The chemically modified sulfamidase did not distribute to the brain parenchyma when given repeatedly by intravenous administration (Rozaklis et al, Exp Neurol 230: 123-130 (2011)).

Thus, there is still a need for effective ERT for treatment of LSDs with neurological engagement. Novel proteins that can be transported across the BBB while remaining functionally active would be of great value in the development of compounds suitable for systemic administration for enzyme/protein replacement therapies for the treatment of LSDs with CNS related pathology.

### DISCLOSURE OF THE INVENTION

It is an object of the present invention to provide novel modified lysosomal proteins allowing development of enzyme replacement therapies for different LSDs.

It is another object of the present invention to provide a novel modified lysosomal protein that may be transported across the blood brain barrier in mammals. In addition, said protein would advantageously have biological activity in the brain of said mammal, such as enzymatic (catalytic) activity in the brain of the mammal.

Yet another object of the present invention is to provide a novel modified lysosomal protein that has catalytic activity in peripheral tissue, in particular a peripheral tissue involved in a peripheral pathology of a LSD.

Yet another object of the present invention is to provide a novel modified lysosomal protein exhibiting improved quality and stability, such as improved structural integrity compared to lysosomal proteins modified according to prior art methods.

These and other objects, which will be apparent to a skilled person from the present disclosure, are achieved by the different aspects of the invention as defined in the appended claims and as generally disclosed herein.

There is, in one aspect of the invention, provided a modified lysosomal protein having a reduced content of unmodified glycan moieties, characterized in that no more than 50% of the unmodified glycan moieties remains intact as compared to an unmodified form of the lysosomal protein, said protein thereby having a reduced activity for glycan recognition receptors, provided that said protein is not sulfamidase. In one embodiment, said protein is not β-glucuronidase. In another embodiment, said protein is not tripeptidyl peptidase 1 (TPP1). In another embodiment, said protein is not alpha L-iduronidase.

By glycan recognition receptors is meant receptors that recognize and bind lysosomal proteins mainly via glycan moieties of the lysosomal proteins. Such receptors can, in addition to the mannose 6-phosphate receptors, be exemplified by the mannose receptor, which selectively binds proteins where glycans exhibit exposed terminal mannose residues. Lectins constitute another large family of glycan recognition receptors which can be exemplified by the terminal galactose recognizing asialoglycoprotein receptor 1 recognizing terminal galactose residues on glycans.

Unmodified or natural glycan moieties should in this respect be understood as glycan moieties naturally occurring in lysosomal protein that are post-translationally modified in the endoplasmatic reticulum and golgi compartments of eukaryotic cells. When unmodified or natural glycan moieties are described as being absent, or when a relative content of glycan moieties is given, this means that intact (or complete) natural glycan moieties cannot be detected. As demonstrated in the appended Examples, relative quantification of glycopeptides may be based on LC-MS and peak areas from reconstructed ion chromatograms. Alternative quantification methods are known to the person skilled in the art.

The modified lysosomal protein according to the invention is thus modified in that natural glycan moieties have been removed. In particular, said lysosomal protein is modified in that epitopes for glycan recognition receptors have been removed from the glycan moieties. Epitopes for glycan recognition receptors should herein be understood as representing (part of) glycan moieties recognized by such receptors and can structurally be described as a sugar moiety of mannose, mannose 6 phosphate, n-acetylglucosamine or galactose origin in the terminal end of a N-glycan. The at least partial absence of natural or unmodified glycan moieties reduces the activity of the modified lysosomal protein with respect to glycan recognition receptors. As a consequence, the receptor mediated endocytosis of the modified lysosomal protein in peripheral tissue might be reduced, which in turn may result in a reduced clearance of the modified protein from plasma when it e.g. is administrated intravenously to a mammal. As demonstrated for certain exemplary lysosomal proteins in the appended examples, a modified lysosomal protein as described herein is less prone to cellular uptake which is a consequence of removal of epitopes for glycan recognition receptors such as the two mannose-6 phosphate receptors (M6PR) (see Example 5 and 6).

From a dosing perspective, reduced clearance of modified lysosomal protein may advantageously allow for development of long-acting medicaments that can be administered to patients less frequently. In addition, modification of said protein may also allow for distribution of the modified lysosomal protein to the CNS. The modified protein as described herein may be transported across the blood brain barrier and into the brain of a mammal where it has biological activity. This advantageous property of the modified protein could potentially improve clinical outcome in a multitude of LSDs.

In one embodiment, no more than 45% of the unmodified glycan moieties remains compared to an unmodified form of the lysosomal protein, such as no more than 40%, no more than 35%, no more than 30%, no more than 35%, no more than 30%, no more than 25%, no more than 20%, no more than 15%, no more than 10%, no more than 5%, no more than 1% of the glycan moieties remains from an unmodified form of the lysosomal protein. Thus, in some embodiments, the modified lysosomal protein comprises substantially no intact natural or unmodified glycan moieties and consequently substantially no epitopes for glycan recognition receptors. This could be understood as an almost complete absence of glycan recognition epitopes. In preferred embodiments, the modified lysosomal protein comprises no (detectable) epitopes for glycan recognition receptors. The in some cases almost complete absence of said epitopes might further reduce the activity of the modified protein with respect to glycan recognition receptors and prolong plasma half life. This is probably at least partly due to the inhibition of receptor mediated uptake in peripheral tissue following chemical modification of protein (as demonstrated in the cellular uptake studies of Example 5).

In particular, the modified lysosomal protein comprises no (detectable) mannose-6-phosphate moieties, mannose moieties, n-acetylglucosamine moieties or galactose moieties that constitute epitopes for the endocytic M6PR type 1 and 2, the mannose receptor, n-acetylglucosamine binding lectins and the galactose receptor, respectively. As defined above, said epitopes, which are found on natural or unmodified glycan moieties, may be selected from mannose-6-phosphate moieties, mannose moieties, n-acetylglucosamine moieties and galactose moieties. In particular embodiments, these are absent from the modified lysosomal protein as disclosed herein.

Said natural glycan moieties of the modified lysosomal protein may be at least partly absent on the modified lysosomal protein as accounted for above. This absence may correspond to disruption, consisting of single bond breaks and double bond breaks, within the natural glycan moieties in said modified lysosomal protein. Glycan disruption by single bond break may typically be predominant. In particular, natural glycan moieties of said lysosomal protein may be disrupted by single bond breaks and double bond breaks, wherein the extent of single bond breaks may be at least 60% in oligomannose glycans. In particular, the extent of single bond breaks may be at least 65%, such as at least 70%, such as at least 75%, such as at least 80%, such as at least 82%, such as at least 85% in the oliogomannose type of glycans. The extent of single bond breaks vs double bond breaks may be determined as described in Examples 9 and 10 for an exemplary protein (sulfamidase).

In one embodiment, said modified lysosomal protein has a molecular weight of more than 95% of that of the corresponding unmodified lysosomal protein, such as more than 96% of that of the corresponding unmodified lysosomal protein, such as more than 97% of that of the corresponding unmodified lysosomal protein, such as more than 98% of that of the corresponding unmodified lysosomal protein, such as more than 99% of that of the corresponding unmodified lysosomal protein. In appended Example 4 it is shown that specific examples of the modified lysosomal proteins according to the invention are undistinguishable from the corresponding unmodified lysosomal proteins in an SDS-PAGE analysis, suggesting mainly single bond breaks, which is depicted in FIG. 8A. In appended Example 2 it is shown that lysosomal proteins modified according to the known method is smaller than the corresponding unmodified lysosomal proteins in an SDS-PAGE analysis, suggesting a higher extent of double bond breaks, which is depicted in FIG. 8A.

In one embodiment of the aspects disclosed herein, said glycan moieties are absent from at least one N-glycosylation site of said modified lysosomal protein, such as at least two, at least three, at least four, at least five, at least six, at least seven, at least eight, at least nine, at least ten, at least eleven, at least twelve, at least thirteen of the N-glycosylation sites of said lysosomal protein, preferably said glycan moieties are absent from all N-glycosylation sites. For example, this means that for a lysosomal protein having two N-glycosylation sites, at least one of the two sites lacks an intact or complete glycan moiety.

In one embodiment of the aspects disclosed herein, said modified lysosomal protein is present in a non-covalently linked form. Advantageously, said lysosomal protein has been modified without causing aggregation of the protein and/or without causing cleavage of the protein backbone into smaller peptide fragments.

In one embodiment, the modified lysosomal protein has retained catalytic activity, such as a retained catalytic activity of at least 50% of that of the corresponding unmodified lysosomal protein, such as at least 60%, at least 70%, at least 80% or at least 90% of that of the corresponding unmodified lysosomal protein. The catalytic activity may be an in vitro or in vivo catalytic activity. A method for measuring catalytic activity in vitro and a modified lysosomal protein having at least 50% catalytic activity is disclosed in Example 12.

Lysosomal proteins are usually rapidly cleared from circulation when administrated by intravenous injection. As described above, cellular uptake from the extracellular compartment is facilitated by receptors recognising the characteristic mannose and mannose 6-phosphate rich glycans of lysosomal proteins. Thus, distribution of lysosomal proteins is typically controlled by the density of these receptors on different cells. While the mannose recognizing receptors are abundantly present on tissue-resident macrophages and sinusoidal endothelial cells in the liver, the cation independent mannose 6-phosphate receptor is abundant on hepatocytes. Consequently, a major part of the dose of an intravenously administrated therapeutic enzyme may distribute to the liver, which is sub-optimal for most therapeutic applications. For example, the two therapeutic α-galactosidase A preparations used as treatment for Fabry disease both show 60-70% of the dose distributed to liver after a single dose in mice (Lee et al, Glycobiology 13: 305-313 (2003). In contrast, cells in tissues that are not very well suplied by blood and/or have low abundance of receptors are not sufficiently targeted via these uptake mechanisms. By preventing rapid uptake via the glycan-dependent routes, clearance from the circulation is significantly reduced and other slower processes facilitate uptake into cells that result in a different distribution profile. This may enable distribution of therapeutic modified lysosomal proteins to cells of tissues that are poorly exposed to unmodified lysosomal enzymes. In particular embodiments, the modified lysosomal proteins as disclosed herein may provide a better distribution in joints, connective tissue, cartilage and bone, when administrated by intravenous infusion. Also skeletal muscle, heart and lung may be better targeted. These are all tissues where a severe pathology is commonly manifested as a consequence of lysosomal storage.

In one embodiment, said modified lysosomal protein distributes to peripheral tissue when administered to a mammal. Examples of peripheral tissue are given above. Moreover, said lysosomal protein may display (retained) biologic activity, such as retained enzymatic or catalytic activity, in said peripheral tissue.

In some embodiments, the modified lysosomal protein according to aspects described herein may distribute to the brain when administered to a mammal, and may also display (retained) biological activity, such as retained enzymatic or catalytic activity, in the brain of said mammal. In one embodiment, the modified lysosomal protein has catalytic activity in the brain.

By retained biological activity is meant that the biological activity of the modified lysosomal protein is retained at least partly from an unmodified form of the lysosomal protein. In order to not completely lose activity of a lysosomal protein upon modification, modification has to be carried out carefully. Modification cannot alter the functional epitope or the active site of the protein such that the modified protein becomes inactive. Thus, the modified lysosomal protein as disclosed herein may affect lysosomal storage in the brain, visceral organs or peripheral tissue of mammals, such as to decrease lysosomal storage, for example lysosomal storage of lipids, GAGs, glycolipids, glycoprotein, amino acids or glycogen.

In particular embodiments, wherein the modified lysosomal protein is a modified sulfatase, the retained catalytic activity may for instance depend on level of preservation versus modification of a catalytic amino acid residue at the active site of sulfatase. Sulfatases are a family of proteins of common evolutionary origin that catalyze the hydrolysis of sulfate ester bonds from a variety of substrates. Thus, “catalytic activity” of a modified sulfatase as used herein may refer to hydrolysis of sulfate ester bonds, preferably in lysosomes of peripheral tissue and/or in lysosomes in the brain of a mammal. Catalytic activity of modified sulfatase may thus result in reduction of lysosomal storage, such as storage of GAGs, e.g. dermatan sulfate, chondroitin sulfate and heparan sulfate, in the brain of a mammal suffering from a lysosomal storage disease. Catalytic activity can for example be measured in an animal model, for example as described in Example 7. Glycan modification of sulfamidase, which is an exemplary sulfatase, has been disclosed in the prior art (Rozaklis et al, supra). The known method for modifying sulfamidase however resulted in a modified sulfamidase lacking catalytic activity in the brain of mice. Thus, this shows that modification of an enzyme has to be carefully performed in order not to jeopardize catalytic activity. The active site of sulfatases typically contains a conserved cysteine that is post-translationally modified to a Ca-formylglycine (FGly). This reaction takes place in the endoplasmic reticulum by the FGly generating enzyme. This FGly resuidue seems necessary for the enzyme to be active. Notably, mutation of the conserved cysteine to a serine (Ser) in arylsulfatase A and B prevents FGly formation and yields inactive enzymes (Recksiek et al, J Biol Chem 13; 273(11):6096-103 (1998)). When preservation of active site of a sulfatase is discussed herein, it should primarily be understood as preservation of the post-translational FGly in said sulfatase.

In one embodiment, the modified lysosomal protein is a lysosomal protein lacking transmembrane helices and having at least one N-glycosylation site. Examples of such lysosomal proteins are listed in the table below:

TABLE I
Non-limiting list of lysosomal proteins

| | N- | | | |
|---|---|---|---|---|
| Name (EC | Glycosylation | | | SEQ ID |
| number) | sites | Involvement in disease | Protein family | NO |
| Deoxyribonuclease- | N68; N194; | | DNase II | 1 |
| 2-alpha | N248; N272 | | | |
| (EC 3.1.22.1) | | | | |
| Beta-mannosidase | N11; N18; | Mannosidosis, beta A, | Glycoside | 2 |
| (EC 3.2.1.25) | N60; N263; | lysosomal | hydrolase 2 | |
| | N267; N280; | | | |
| | N285; N746 | | | |
| Ribonuclease T2 | N52; N82; | Leukoencephalopathy, | RNase T2 | 3 |
| (EC 3.1.27.—) | N188 | cystic, without | | |
| | | megalencephaly | | |
| Lysosomal alpha- | N84; N261; | Mannosidosis, alpha B, | Glycoside | 4 |
| mannosidase | N318; N448; | lysosomal | hydrolase 38 | |
| (EC 3.2.1.24) | N596; N602; | | | |
| | N643; N717; | | | |
| | N783; N881; | | | |
| | N940 | | | |
| Tripeptidyl- | N191; N203; | Ceroid lipofuscinosis, | Peptidase | 5 |
| peptidase 1 | N267; N294; | neuronal, 2; | S53 | |
| (EC 3.4.14.9) | N424 | Spinocerebellar ataxia, | | |
| | | autosomal recessive, 7 | | |
| Hyaluronidase-3 | N49; N195 | | Glycoside | 6 |
| (EC 3.2.1.35) | | | hydrolase 56 | |
| Cathepsin L2 | N204; N275 | | Peptidase C1 | 7 |
| (EC 3.4.22.43) | | | | |
| Ceroid- | N84; N97; | Ceroid lipofuscinosis, | CLN5 | 8 |
| lipofuscinosis | N132; N157; | neuronal, 5 | | |
| neuronal protein 5 | N209; N225; | | | |
| | N235; N306 | | | |
| Glucosylceramidase | N19; N59; | Gaucher disease | Glycoside | 9 |
| (EC 3.2.1.45) | N146; N270; | | hydrolase 30 | |
| | N462 | | | |
| Tissue alpha-L- | N210; N237; | Fucosidosis | Glycoside | 10 |
| fucosidase | N351 | | hydrolase 29 | |
| (EC 3.2.1.51) | | | | |
| Myeloperoxidase | N91; N275; | Myeloperoxidase | Peroxidase, | 11 |
| (EC 1.11.2.2) | N307; N343; | deficiency | XPO | |
| | N435; N681 | | subfamily | |
| Alpha- | N108; N161; | Fabry disease | Glycoside | 12 |
| galactosidase A | N184; N377 | | hydrolase 27 | |
| (EC 3.2.1.22) | | | | |
| Beta- | N93; N135; | GM2-gangliosidosis 1 | Glycoside | 13 |
| hexosaminidase | N273 | | hydrolase 20 | |
| subunit alpha | | | | |
| (EC 3.2.1.52) | | | | |
| Cathepsin D | N116; N245 | Ceroid lipofuscinosis, | Peptidase A1 | 14 |
| (EC 3.4.23.5) | | neuronal, 10 | | |
| Prosaposin | N64; N85; | Combined saposin | Saposin | 15 |
| | N199; N316; | deficiency; | superfamily | |
| | N410 | Leukodystrophy | | |
| | | metachromatic due to | | |
| | | saposin-B deficiency; | | |
| | | Gaucher disease, | | |
| | | atypical, due to saposin C | | |
| | | deficiency; Krabbe | | |
| | | disease, atypical, due to | | |
| | | saposin A deficiency; | | |
| | | Defects in PSAP saposin- | | |
| | | D region are found in a | | |
| | | variant of Tay-Sachs | | |
| | | disease | | |
| Beta- | N42; N100; | GM2-gangliosidosis 2 | Glycoside | 16 |
| hexosaminidase | N148; N281; | | hydrolase 20 | |
| subunit beta | N285 | | | |
| (EC 3.2.1.52) | | | | |
| Cathepsin L1 | N204 | | Peptidase C1 | 17 |
| (EC 3.4.22.15) | | | | |
| Cathepsin B | N175 | | Peptidase C1 | 18 |
| (EC 3.4.22.1) | | | | |
| Beta- | N151; N250; | Mucopolysaccharidosis 7 | Glycoside | 19 |
| glucuronidase (EC | N398; N609 | | hydrolase 2 | |
| 3.2.1.31) | | | | |
| Pro-cathepsin H | N79; N208 | | Peptidase C1 | 20 |
| (EC 3.4.22.16) | | | | |
| Non-secretory | N17; N59; | | Pancreatic | 21 |
| ribonuclease | N65; N84; | | ribonuclease | |
| (EC 3.1.27.5) | N92 | | | |
| Lysosomal alpha- | N113; N206; | Glycogen storage | Glycoside | 22 |
| glucosidase | N363; N443; | disease 2 | hydrolase 31 | |
| (EC 3.2.1.20) | N625; N855; | | | |
| | N898 | | | |
| Lysosomal | N117; N305 | Galactosialidosis | Peptidase | 23 |
| protective protein | | | S10 | |
| (EC 3.4.16.5) | | | | |
| Gamma-interferon- | N37; N69; | | GILT | 24 |
| inducible | N82 | | | |
| lysosomal thiol | | | | |
| reductase | | | | |
| (EC 1.8.—.—) | | | | |
| Tartrate-resistant | N95; N126 | Spondyloenchondro- | Metallophosphoesterase | 25 |
| acid phosphatase | | dysplasia with immune | superfamily, | |
| type 5 | | dysregulation | Purple acid | |
| (EC 3.1.3.2) | | | phosphatase | |
| Arylsulfatase A | N140; N166; | Leukodystrophy | Sulfatase | 26 |
| (EC 3.1.6.8) | N332 | metachromatic | | |
| Prostatic acid | N62; N188; | | Histidine acid | 27 |
| phosphatase | N301 | | phosphatase | |
| (EC 3.1.3.2) | | | | |
| N- | N75; N81; | Mucopolysaccharidosis | Sulfatase | 28 |
| acetylglucosamine- | N147; N162; | 3D | | |
| 6-sulfatase | N174; N243; | | | |
| (EC 3.1.6.14) | N281; N326; | | | |
| | N351; N369; | | | |
| | N386; N413; | | | |
| | N444 | | | |
| Arylsulfatase B | N152; N243; | Mucopolysaccharidosis 6 | Sulfatase | 29 |
| (EC 3.1.6.12) | N255; N330; | | | |
| | N390; N422 | | | |
| Beta-galactosidase | N3; N224; | GM1-gangliosidosis 1-3; | Glycoside | 30 |
| (EC 3.2.1.23) | N441; N475; | Mucopolysaccharidosis | hydrolase 35 | |
| | N519; N522; | 4B | | |
| | N532 | | | |
| Alpha-N- | N107; N160; | Schindler disease; | Glycoside | 31 |
| acetylgalactosaminidase | N184; N342; | Kanzaki disease | hydrolase 27 | |
| (EC | N368 | | | |
| 3.2.1.49) | | | | |
| Sphingomyelin | N40; N129; | Niemann-Pick disease A | Acid | 32 |
| phosphodiesterase | N289; N349; | & B | sphingomyelinase | |
| (EC 3.1.4.12) | N474 | | | |
| Ganglioside GM2 | N40 | GM2-gangliosidosis AB | MD-2-related | 33 |
| activator | | | lipid- | |
| | | | recognition | |
| | | | domain | |
| N(4)-(beta-N- | N15; N285 | Aspartylglucosaminuria | Ntn-hydrolase | 34 |
| acetylglucosaminyl)- | | | | |
| L-asparaginase | | | | |
| (EC 3.5.1.26) | | | | |
| Iduronate 2- | N90; N119; | Mucopolysaccharidosis 2 | Sulfatase | 35 |
| sulfatase | N221; N255; | | | |
| (EC 3.1.6.13) | N300; N488; | | | |
| | N512 | | | |
| Cathepsin S | N88 | | Peptidase C1 | 36 |
| (EC 3.4.22.27) | | | | |
| N-acetylgalactos- | N178; N397 | Mucopolysaccharidosis | Sulfatase | 37 |
| amine-6-sulfatase | | 4A | | |
| (EC 3.1.6.4) | | | | |
| Alpha-L- | N83; N163; | Mucopolysaccharidosis 1 | Glycoside | 38 |
| iduronidase (EC | N309; N345; | | hydrolase 39 | |
| 3.2.1.76) | N388; N424 | | | |
| Lysosomal acid | N13; N49; | Wolman disease; | AB hydrolase | 39 |
| lipase/cholesteryl | N78; N138; | Cholesteryl ester storage | superfamily, | |
| ester hydrolase | N250; N298 | disease | Lipase | |
| (EC 3.1.1.13) | | | | |
| Lysosomal Pro-X | N26; N80; | | Peptidase | 40 |
| carboxypeptidase | N296; N315; | | S28 | |
| (EC 3.4.16.2) | N324; N394 | | | |
| Cathepsin O | N39; N82 | | Peptidase C1 | 41 |
| (EC 3.4.22.42) | | | | |
| Cathepsin K | N88 | Pycnodysostosis | Peptidase C1 | 42 |
| (EC 3.4.22.38) | | | | |
| Palmitoyl-protein | N170; N185; | Ceroid lipofuscinosis, | Palmitoyl- | 43 |
| thioesterase 1 | N205 | neuronal, 1 | protein | |
| (PPT-1) (EC | | | thioesterase | |
| 3.1.2.22) | | | | |
| Sulfamidase | N21; N122; | Mucopolysaccharidosis | Sulfatase | 44 |
| (EC 3.10.1.1) | N131; N244; | 3A | | |
| | N393 | | | |
| Arylsulfatase D | N28; N95; | | Sulfatase | 45 |
| (ASD) (EC 3.1.6.—) | N314 | | | |
| Dipeptidyl | N5; N29; | Papillon-Lefevre | Peptidase C1 | 46 |
| peptidase 1 (EC | N95; N252 | syndrome; Haim-Munk | | |
| 3.4.14.1) | | syndrome; Periodontititis, | | |
| | | aggressive, 1 | | |
| Alpha-N- | N238; N249; | Mucopolysaccharidosis | Glycoside | 47 |
| acetylglucosaminidase | N412; N480; | 3B | hydrolase 89 | |
| (EC 3.2.1.50) | N503; N509 | | | |
| Galactocerebrosidase | N101; N337; | Leukodystrophy, globoid | Glycoside | 48 |
| (EC | N361; N514; | cell | hydrolase 59 | |
| 3.2.1.46) | N517; N560 | | | |
| Epididymal | N39; N116 | Niemann-Pick disease C2 | NPC2 | 49 |
| secretory protein | | | | |
| E1 | | | | |
| Di-N- | N155; N190; | | Glycoside | 50 |
| acetylchitobiase | N224; N261 | | hydrolase 18 | |
| (EC 3.2.1.—) | | | | |
| N- | N9; N79; | | Acid | 51 |
| acylethanolamine- | N281; N305 | | ceramidase | |
| hydrolyzing acid | | | | |
| amidase (EC | | | | |
| 3.5.1.—) | | | | |
| Hyaluronidase-1 | N78; N195; | Mucopolysaccharidosis 9 | Glycoside | 52 |
| (EC 3.2.1.35) | N329 | | hydrolase 56 | |
| Chitotriosidase-1 | N79 | | Glycoside | 53 |
| (EC 3.2.1.14) | | | hydrolase 18, | |
| | | | Chitinase | |
| | | | class II | |
| | | | subfamily | |
| Acid ceramidase | N152; N174; | Farber | Acid | 54 |
| (ACDase) | N238; N265; | lipogranulomatosis; | ceramidase | |
| | N321; N327 | Spinal muscular atrophy | | |
| | | with progressive | | |
| | | myoclonic epilepsy | | |
| Phospholipase B- | N33; N270; | | Phospholipase | 55 |
| like 1 (EC 3.1.1.—) | N328; N373; | | B-like | |
| | N488 | | | |
| Proprotein | N503 | Hypercholesterolemia, | Peptidase S8 | 56 |
| convertase | | autosomal dominant, 3 | | |
| subtilisin/kexin | | | | |
| type 9 (EC 3.4.21.—) | | | | |
| Group XV | N66; N240; | | AB hydrolase | 57 |
| phospholipase A2 | N256; N365 | | superfamily, | |
| (EC 2.3.1.—) | | | Lipase | |
| Putative | N47; N69; | | Phospholipase | 58 |
| phospholipase B- | N190; N395; | | B-like | |
| like 2 (EC 3.1.1.—) | N424; N474 | | | |
| Deoxyribonuclease- | N54; N76; | | DNase II | 59 |
| 2-beta (EC | N92; N251 | | | |
| 3.1.22.1) | | | | |
| Gamma-glutamyl | N92; N139; | | Peptidase | 60 |
| hydrolase | N179; N283 | | C26 | |
| (EC 3.4.19.9) | | | | |
| Arylsulfatase G | N101; N199; | | Sulfatase | 61 |
| (EC 3.1.6.—) | N340; N481 | | | |
| L-amino-acid | N33; N113; | | Flavin | 62 |
| oxidase | N199; N538 | | monoamine | |
| (EC 1.4.3.2) | | | oxidase, | |
| | | | FIG. 1 | |
| | | | subfamily | |
| Sialidase-1 | N139; N296; | Sialidosis | Glycoside | 63 |
| (EC 3.2.1.18) | N305 | | hydrolase 33 | |
| Legumain | N74; N150; | | Peptidase | 64 |
| (EC 3.4.22.34) | N246; N255 | | C13 | |
| Sialate O- | N84; N115; | Autoimmune disease 6 | SGNH | 65 |
| acetylesterase | N244; N267; | | hydrolase- | |
| (EC 3.1.1.53) | N378; N399 | | type esterase | |
| | | | domain | |
| Thymus-specific | N46; N148; | | Peptidase | 66 |
| serine protease | N297 | | S28 | |
| (EC 3.4.—.—) | | | | |
| Cathepsin Z | N161; N201 | | Peptidase C1 | 67 |
| (EC 3.4.18.1) | | | | |
| Cathepsin F | N141; N176; | Ceroid lipofuscinosis, | Peptidase C1 | 68 |
| (EC 3.4.22.41) | N348; N359; | neuronal, 13 | | |
| | N421 | | | |
| Prenylcysteine | N169; N296; | | Prenylcysteine | 69 |
| oxidase 1 | N326 | | oxidase | |
| (EC 1.8.3.5) | | | | |
| Dipeptidyl | N29; N65; | | Peptidase | 70 |
| peptidase 2 (EC | N294; N335; | | S28 | |
| 3.4.14.2) | N342; N407 | | | |
| Lysosomal | N33; N163; | | Palmitoyl- | 71 |
| thioesterase PPT2 | N179; N218; | | protein | |
| (EC 3.1.2.—) | N262 | | thioesterase | |
| Heparanase | N127; N143; | | Glycoside | 72 |
| (EC 3.2.1.166) | N165; N182; | | hydrolase 79 | |
| | N203; N424 | | | |
| Carboxypeptidase | N41; N159; | | Peptidase | 73 |
| Q (EC 3.4.17.—) | N333; N336; | | M28 | |
| | N376 | | | |
| Sulfatase- | N108 | Multiple sulfatase | Sulfatase- | 74 |
| modifying factor 1 | | deficiency | modifying | |
| (EC 1.8.99.—) | | | factor | |

In Table I a number of lysosomal proteins are listed. Some of the proteins might be known under other names. It should be understood that the protein listing above also encompasses any and all alternative names.

In one embodiment, the modified lysosomal protein is selected from the group consisting of deoxyribonuclease-2-alpha; beta-mannosidase; ribonuclease T2; lysosomal alpha-mannosidase (Laman); tripeptidyl-peptidase 1 (TPP-1); hyaluronidase-3 (Hyal-3); cathepsin L2; ceroid-lipofuscinosis neuronal protein 5; glucosylceramidase; tissue alpha-L-fucosidase; myeloperoxidase (MPO); alpha-galactosidase A; beta-hexosaminidase subunit alpha; cathepsin D; prosaposin; beta-hexosaminidase subunit beta; cathepsin L1; cathepsin B; beta-glucuronidase; pro-cathepsin H; cathepsin H; non-secretory ribonuclease; lysosomal alpha-glucosidase; lysosomal protective protein; gamma-interferon-inducible lysosomal thiol reductase; tartrate-resistant acid phosphatase type 5 (TR-AP); arylsulfatase A (ASA); prostatic acid phosphatase (PAP); N-acetylglucosamine-6-sulfatase; arylsulfatase B (ASB); beta-galactosidase; alpha-N-acetylgalactosaminidase; sphingomyelin phosphodiesterase; ganglioside GM2 activator; N(4)-(beta-N-acetylglucosaminyl)-L-asparaginase; iduronate 2-sulfatase; cathepsin S; N-acetylgalactosamine-6-sulfatase; alpha-L-iduronidase; lysosomal acid lipase/cholesteryl ester hydrolase (Acid cholesteryl ester hydrolase) (LAL); lysosomal Pro-X carboxypeptidase; cathepsin O; cathepsin K; palmitoyl-protein thioesterase 1 (PPT-1); sulfamidase; arylsulfatase D (ASD); dipeptidyl peptidase 1; alpha-N-acetylglucosaminidase; galactocerebrosidase (GALCERase); epididymal secretory protein E1; di-N-acetylchitobiase; N-acylethanolamine-hydrolyzing acid amidase; hyaluronidase-1 (Hyal-1); chitotriosidase-1; acid ceramidase (AC); phospholipase B-like 1; proprotein convertase subtilisin/kexin type 9; group XV phospholipase A2; putative phospholipase B-like 2; deoxyribonuclease-2-beta; gamma-glutamyl hydrolase; arylsulfatase G (ASG); L-amino-acid oxidase (LAAO) (LAO); sialidase-1; legumain; sialate O-acetylesterase; thymus-specific serine protease; cathepsin Z; cathepsin F (CATSF); prenylcysteine oxidase 1; dipeptidyl peptidase 2; lysosomal thioesterase PPT2 (PPT-2); heparanase; carboxypeptidase Q; β-glucuronidase, and sulfatase-modifying factor 1.

In certain embodiments of aspects disclosed herein, said modified lysosomal protein is a sulfatase. Said sulfatase preferably has a FGly residue at its active site. In some embodiments, said sulfatase is thus selected from arylsulfatase A; N-acetylglucosamine-6-sulfatase, arylsulfatase B; iduronate 2-sulfatase; N-acetylgalactosamine-6-sulfatase; sulfamidase; arylsulfatase D, and arylsulfatase G. In particular, said sulfatase is arylsulfatase A; N-acetylglucosamine-6-sulfatase; arylsulfatase B; iduronate 2-sulfatase; N-acetylgalactosamine-6-sulfatase or sulfamidase. Preferably, said sulfatase is arylsulfatase A. Sulfamidase might in some embodiments be excluded.

In embodiments of aspects disclosed herein, said modified lysosomal protein is a glycoside hydrolase. In some embodiments, said glycoside hydrolase is selected from alpha-galactosidase A; tissue alpha-L-fucosidase; glucosylceramidase; lysosomal alpha-glucosidase; beta-galactosidase; beta-hexosaminidase subunit alpha; beta-hexosaminidase subunit beta; galactocerebrosidase; lysosomal alpha-mannosidase; beta-mannosidase; alpha-L-iduronidase; alpha-N-acetylglucosaminidase; beta-glucuronidase; hyaluronidase-1; alpha-N-acetylgalactosaminidase; sialidase-1; di-N-acetylchitobiase; chitotriosidase-1; hyaluronidase-3, and heparanase. Preferably, said glycoside hydrolase is alpha-L-iduronidase or lysosomal alpha-mannosidase. Preferably, said glycoside hydrolase is lysosomal alpha-mannosidase.

In embodiments of aspects disclosed herein, said modified lysosomal protein is a protease. In some embodiments, said protease is selected from cathepsin D; cathepsin L2; cathepsin L1; cathepsin B; pro-cathepsin H; cathepsin S; cathepsin O; cathepsin K; dipeptidyl peptidase 1; cathepsin Z; cathepsin F; legumain; gamma-glutamyl hydrolase; tripeptidyl-peptidase 1; carboxypeptidase Q; lysosomal protective protein; lysosomal pro-X carboxypeptidase; thymus-specific serine protease; dipeptidyl peptidase 2, and proprotein convertase subtilisin/kexin type 9. In one embodiment, said protease is tripeptidyl-peptidase 1. In another embodiment, tripeptidyl-peptidase is excluded from the group of proteases listed above.

In one embodiment of the aspects as disclosed herein, said modified lysosomal protein comprises polypeptide consisting of an amino acid sequence selected from any one of SEQ ID NO:1-74, or a polypeptide having at least 90% sequence identity with an amino acid sequence selected from SEQ ID NO:1-74. In a non-limiting example, said polypeptide has at least 95% sequence identity with an amino acid sequence selected from SEQ ID NO:1-74, such as at least 98% sequence identity with an amino acid sequence selected from SEQ ID NO:1-74, such as at least 99% sequence identity with an amino acid sequence selected from SEQ ID NO:1-74.

In a specific embodiment, said modified lysosomal protein is a modified sulfatase and comprises a polypeptide consisting of an amino acid sequence selected from any one of SEQ ID NO:26; 28; 29; 35; 37; 44; 45, and 61. In a preferred embodiment, said polypeptide has an amino acid sequence is selected from SEQ ID NO: 26; SEQ ID NO: 28; SEQ ID NO: 29; SEQ ID NO: 35; SEQ ID NO: 37 and SEQ ID NO: 44. In a preferred embodiment, said polypeptide has an amino acid sequence as set out in SEQ ID NO:26.

In another embodiment, said modified lysosomal protein is a modified glycoside hydrolase and comprises a polypeptide consisting of an amino acid sequence selected from any one of SEQ ID NO: 12; SEQ ID NO: 10; SEQ ID NO: 9; SEQ ID NO: 22; SEQ ID NO: 30; SEQ ID NO: 13; SEQ ID NO: 16; SEQ ID NO: 48; SEQ ID NO: 4; SEQ ID NO: 2; SEQ ID NO: 38; SEQ ID NO: 47; SEQ ID NO: 19; SEQ ID NO: 52; SEQ ID NO: 31; SEQ ID NO: 63; SEQ ID NO: 50; SEQ ID NO: 53; SEQ ID NO: 6, and SEQ ID NO: 72. In a preferred embodiment, said polypeptide has an amino acid sequence as set out in SEQ ID NO:4 or SEQ ID NO:38.

In another embodiment, said modified lysosomal protein is a modified protease and comprises a polypeptide consisting of an amino acid sequence selected from any one of SEQ ID NO:14; SEQ ID NO:68; SEQ ID NO:5; SEQ ID NO:23; SEQ ID NO:56; SEQ ID NO:46; SEQ ID NO:42; SEQ ID NO:7; SEQ ID NO:17; SEQ ID NO:18; SEQ ID NO:20; SEQ ID NO:36; SEQ ID NO:41; SEQ ID NO:67; SEQ ID NO:64; SEQ ID NO:60; SEQ ID NO:73; SEQ ID NO:40; SEQ ID NO:66, and SEQ ID NO:70. In a preferred embodiment, said polypeptide has an amino acid sequence as set out in SEQ ID NO:5.

In a further embodiment, said polypeptide may however be extended by one or more C- and/or N-terminal amino acid(s), making the actual modified lysosomal protein sequence longer than the sequence of SEQ ID NO:1-74. Similarly, in other instances the modified lysosomal protein may have an amino acid sequence which is shorter than the amino acid sequence of SEQ ID NO:1-74, the difference in length e.g. being due to deletion(s) of amino acid residue(s) in certain position(s) of the sequence.

In one embodiment, said modified lysosomal protein is isolated.

In one embodiment, said lysosomal protein is a human lysosomal protein.

In one embodiment, said lysosomal protein prior to modification is glycosylated.

In one embodiment, said modified lysosomal protein is recombinant. In particular, lysosomal protein may be recombinantly produced in a continuous human cell line.

In one embodiment, said modified protein is expressed in mammalian, Chinese hamster ovary, plant or yeast cells. The resulting protein is thus, prior to modification, glycosylated by one or more oligomannose N-glycans.

In one aspect, there is provided a composition, comprising modified lysosomal protein having a reduced content of natural or unmodified glycan moieties, characterized in that no more than 50% of the natural or unmodified glycan moieties remains compared to an unmodified form of the lysosomal protein, thereby enabling transportation of said lysosomal protein across the blood brain barrier and into the brain of a mammal where said modified lysosomal protein has biological activity. In one embodiment, said protein is not sulfamidase, β-glucuronidase, or tripeptidyl peptidase 1 (TPP1). In another embodiment, said protein is not alpha L-iduronidase.

In particular embodiments wherein the lysosomal protein is a sulfatase, said composition may be characterized in that a Ca-formylglycine (FGly) to serine (Ser) ratio at the active site of said modified sulfatase is greater than 1. For example, said modified lysosomal protein is a sulfatase comprising a polypeptide consisting of an amino acid sequence as defined in any one of SEQ ID NO:26; 28; 29; 35; 37; 44; 45, and 61; or a polypeptide having at least 90% sequence identity with a polypeptide as defined in SEQ ID NO:26; 28; 29; 35; 37; 44; 45, and 61. Preferably, the FGly to Ser ratio is exceeds 1.5, more preferably it exceeds 2.3, more preferably 4, and most preferably the ratio is around 9. A larger ratio indicates that the catalytic activity of the modified sulfatase to a larger extent may be retained from an unmodified form of the sulfatase.

The advantages disclosed for other aspects also apply to the composition aspect. Similarly, the embodiments disclosed for other aspects also apply to the composition aspect. In particular, the embodiments related to content of glycan moieties, protein activity, and particular examples of lysosomal proteins (see Table I and lists above) are applicable also to this aspect.

In one embodiment of the composition aspect, no more than 10%, such as no more than 7.5%, no more than 5%, no more than 2.5%, no more than 1% (by weight) of said modified lysosomal protein is present in multimeric forms having a molecular weight of above 10¹⁰ kDa.

In one embodiment of the composition aspect, no more than 10% (by weight) of said modified lysosomal protein is present in covalently linked oligomeric forms, said oligomeric forms being selected from dimers, trimers, tetramers, pentamers, hexamers, heptamers and octamers. The presence of oligomeric, multimeric, or aggregated forms, can for example be determined by dynamic light scattering or by size exclusion chromatography. In this context, aggregated forms should be understood as high molecular weight protein forms composed of structures ranging from natively folded to unfolded monomers. Aggregated forms of a protein can enhance immune response to the monomeric form of the protein. The most likely explanation for an enhanced immune response is that the multivalent presentations of antigen cross link B-cell receptors and thus induce an immune response. This is a phenomenon which has been utilized in vaccine production where the antigen is presented to the host in an aggregated form to ensure a high immune response. For therapeutic proteins the dogma is the opposite; any content of high molecular weight forms should be minimized or avoided in order to minimize the immune response (Rosenberg, AAPS J, 8:E501-7 (2006)). Thus, reduction of oligomeric, multimeric and/or aggregate forms may thus provide an enzyme or protein more suitable for use in therapy.

Moreover, the occurrence of even a small amount of aggregated protein in a sample may induce further aggregation of normally folded proteins. The aggregated material generally has no or low remaining activity and poor solubility. The appearance of aggregates can be one of the factors that determine the shelf-life of a biological medicine (Wang, Int J Pharm, 185:129-88 (1999)).

The term “composition” as used herein should be understood as encompassing solid and liquid forms. A composition may preferably be a pharmaceutical composition, suitable for administration to a patient (e.g. a mammal) for example by injection or orally.

In one aspect, there is provided a modified lysosomal protein, wherein said lysosomal protein has been prepared by sequential reaction with an alkali metal periodate and an alkali metal borohydride, thereby modifying epitopes for glycan recognition receptors of the lysosomal protein and reducing the activity of the lysosomal protein with respect to said glycan recognition receptors, while retaining biological activity of said lysosomal protein. The lysosomal protein is thus modified in that its epitopes, or glycan moieties, present in its natural, glycosylated form prior to modification has been essentially inactivated by said modification. The presence of epitopes for glycan recognition receptors have thus been reduced in the modified lysosomal protein. It should be understood that the embodiments, and their advantages, disclosed in relation to the other aspects disclosed herein, such as the aspects related to modified lysosomal protein, composition and method of preparation, are embodiments also of this aspect. In particular, the various method embodiments disclosed below provide further exemplary definition of the preparation of said modified lysosomal protein in terms of specific reaction conditions. Similarly, the embodiments disclosed in relation to the modified lysosomal protein and composition aspects above provide further exemplary definition of the modified lysosomal protein.

There is, in one aspect, provided a method of preparing a modified lysosomal protein, said method comprising: a) reacting a glycosylated lysosomal protein with an alkali metal periodate, and b) reacting said lysosomal protein with an alkali metal borohydride for a time period of no more than 2 h; thereby modifying glycan moieties of the lysosomal protein and reducing the activity of the lysosomal protein with respect to glycan recognition receptors, provided that said protein is not sulfamidase.

There is, in one related aspect, provided a method of preparing a modified lysosomal protein, said method comprising: a) reacting a glycosylated lysosomal protein with an alkali metal periodate for a time period of no more than 4 h, and b) reacting said lysosomal protein with an alkali metal borohydride for a time period of no more than 2 h; thereby modifying glycan moieties of the lysosomal protein and reducing the activity of the lysosomal protein with respect to glycan recognition receptors, provided that said protein is not sulfamidase.

There is, in a related aspect, provided a method of preparing a modified lysosomal protein, said method comprising: a) reacting a glycosylated lysosomal protein with an alkali metal periodate, and b) reacting said lysosomal protein with an alkali metal borohydride, optionally for a time period of no more than 2 h; thereby modifying glycan moieties of the lysosomal protein and reducing the activity of the lysosomal protein with respect to glycan recognition receptors, wherein the active site or functional epitope of said lysosomal protein is made inaccessible to oxidative and/or reductive reactions during at least one of steps a) and b).

The term “functional epitope” should in this context be understood as the part of a protein that has an essential function in the lysosome although the protein has no enzymatic activity. An essential function could be provided e.g. by presenting the substrate to the degrading enzyme, by influencing sorting of enzymes or acting as a binding partner to a functional enzyme. The functional epitope of the protein in question is then defined by the residues of the protein involved in its function, e.g. ligand binding residues or residues involved in protein-protein binding that defines the function of the protein.

The above methods thus provide mild chemical modification of a lysosomal protein that reduces the presence of epitopes for glycan recognition receptors, said epitopes for example being represented by natural or unmodified glycan moieties as described herein. This advantageously may provide a modified lysosomal protein suitable for targeting the brain of a mammal and/or such visceral organs and/or such peripheral tissues where otherwise unmodified lysosomal proteins are poorly distributed. In particular, the method may provide lysosomal proteins with higher exposure in peripheral tissue such as joints, connective tissue, cartilage and bone, when administrated by e.g. intravenous infusion. The mild methods moreover advantageously modify said epitopes without leading to a complete loss of biological activity. In particular embodiments, the mild methods do not modify the functional epitopes of the lysosomal protein such that its biological activity is lost. When said lysosomal protein is a sulfatase, the biological activity may be a catalytic activity which is retained by retaining a FGly at the active site of the modified lysosomal protein. Thus, while improving distribution properties of the protein or enzyme, the methods do not eliminate biological, e.g. catalytic, activity. Further advantages with the modified lysosomal protein prepared by the mild methods are as accounted for above, e.g. for the lysosomal protein and composition aspects.

The methods allow for glycan modification by periodate cleavage of carbon bonds between two adjacent hydroxyl groups of the glycan (carbohydrate) moieties. In general, periodate oxidative cleavage occurs where there are vicinal diols present. The diols have to be present in an equatorial—equatorial or axial—equatorial position. If the diols are present in a rigid axial-axial position no reaction takes place (Kristiansen et al, Car. Res (2010)). The periodate treatment will break the bond between C2 and C3 and/or C3 and C4 of the M6P moiety, thus yielding a structure that is incapable of binding to a M6P-receptor. In general, other terminal hexoses will also be processed in a similar way. Non-terminal 1-4 linked residues are cleaved between C2 and C3 only, whereas non-terminal (1-3) linked residues are resistant to cleavage. In FIG. 7, the points of possible modification are marked with asterisks in the three general types of N-glycans; oligomannose, complex and hybrid N-glycans. As further demonstrated in appended Examples, the methods as disclosed herein provides a modified lysosomal protein in which the natural glycan moieties have been disrupted by a limited number of bond breaks. Typically, modification by use of the known method gives rise to more extensive disruption, as has been demonstrated in comparative experiments for the polypeptide sulfamidase. Periodate used in step a) may disrupt the structure of the glycan moieties naturally occurring on lysosomal protein. The remaining glycan structure of the modified lysosomal protein may have been at least partially disrupted in that at least one periodate catalyzed cleavage, i.e. at least one single bond break, has occurred in each of the naturally occurring glycan moieties. The presently disclosed methods may predominantly result in a single-type of bond breaks in sugar moieties of the glycan moieties of the lysosomal protein (see FIG. 8). The difference between the known method and the methods as disclosed herein with respect to the tendency of double bond breaks vs single bond breaks can for example be observed on SDS-PAGE where a tendency towards predominantly double bond breaks leads to a more pronounced loss in molecular weight of the monomeric protein. In a modified protein wherein predominantly single bond breaks have occurred in the glycan moieties, the loss in molecular weight of the monomeric protein is less pronounced or even negligible as compared to an unmodified form of the protein. A repertoire of modified glycan moieties predominantly exhibiting single-type of bond breaks may in turn be beneficial for the distribution and activity of the lysosomal protein in the brain in a living animal after intravenous administration.

The methods of preparing a modified lysosomal protein, and the modified lysosomal protein as described herein, are improved over prior art methods and compounds. Primarily, the novel modified lysosomal protein may be distributed to and display biological activity in the mammalian brain. Examples 2 and 4 moreover provide comparisons between lysosomal proteins modified according to known methods and lysosomal proteins modified according to the methods as disclosed herein. The results in these examples show that lysosomal proteins modified according to known methods display alterations of the amino acid sequence, polypeptide chain cleavages and protein aggregation. It has in particular been observed that in sulfatases, containing a catalytic FGly residue at the active site, the known method of modification leads to conversion of the FGly residue to a Ser residue. Thus, the methods as disclosed herein moreover may provide a modified lysosomal protein with improved quality and stability in terms of e.g. structural integrity.

In one embodiment of the method aspects, said alkali metal periodate oxidizes cis-glycol groups of the glycan moieties to aldehyde groups.

In one embodiment of the method aspects, said alkali metal borohydride reduces said aldehydes to alcohols.

In one embodiment of the method aspects, step a) and step b) are performed in sequence without performing an intermediate step. By performing step b) immediately after step a), or after an optional quenching step a2) as described below, any intermediate step such as to remove reactive reagents by e.g. dialysis, ultrafiltration, precipitation or buffer exchange, is omitted, and long exposure of lysosomal protein to reactive aldehyde intermediates is thus avoided. Proceeding with step b) after step a), or optionally a2), the overall reaction duration is also advantageously reduced.

In the following paragraphs, specific embodiments of step a) are disclosed. It should be understood that unless defined otherwise specific embodiments of aspects disclosed herein can be combined.

In one embodiment, said alkali metal periodate is sodium meta-periodate.

In one embodiment, said reaction of step a) is performed for a time period of no more than 4 h, such as no more than 3 h, such as no more than 2 h, such as no more than 1 h, such as around 0.5 h. In certain embodiments, the reaction of step a) is performed for no more than 0.5 h, such as around 20 minutes. The reaction preferably has a duration of around 3 h, 2 h, 1 h, or less than 1 h. A duration of step a) of no more than 4 hours may efficiently inactivate epitopes for glycan recognition receptors. In addition, a relatively limited duration of no more than 4 h is hypothesized to give rise to a limited degree of strand-breaks of the polypeptide chain.

In one embodiment, said periodate is used at a (final) concentration of no more than 20 mM, such as no more than 15 mM, such as around 10 mM. The periodate may be used at a concentration of 8-20 mM, preferably around 10 mM. Alternatively, periodate is used at a concentration of less than 20 mM, such as between 10 and 19 mM. Lower concentration of alkali metal periodate, such as sodium meta-periodate, may reduce the degree of strand-breaks of the polypeptide chain, as well as associated oxidation on amino acids side-chains, such as oxidation of methionine residues.

In one embodiment, said reaction of step a) is performed at ambient temperature, and preferably at a temperature of between 0 and 22° C. In a preferred embodiment, the reaction of said step a) is performed at a temperature of 0-8° C., such as at a temperature of 0-4° C. In a preferred embodiment, the reaction of step a) is performed at a temperature of around 8° C., at a temperature of around 4° C. or at a temperature of around 0° C.

In one embodiment, said reaction of step a) is performed at a pH of 3 to 7. This pH should be understood as the pH at the initiation of the reaction. In particular embodiments, the pH used in step a) is 3-6, such as 4-5. In specific embodiments, the pH used in step a) is around 6, around 5, or around 4. By lowering the pH of step a), the concentration of periodate or the reaction time of step a) may be reduced.

In one embodiment, said periodate is sodium meta-periodate and is used at a (final) concentration of no more than 20 mM, such as no more than 15 mM, such as around 10 mM. In one embodiment, said sodium meta-periodate is used at a concentration of 8-20 mM. In preferred embodiments, sodium meta-periodate is used at a concentration of around 10 mM.

In one embodiment, said periodate is sodium meta-periodate and is used at a (final) concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h. A concentration of 20 mM periodate and a reaction duration of no more than 4 h may advantageously result in less strand-break and oxidation.

In one embodiment, said periodate is sodium meta-periodate and is used at a (final) concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h at a temperature of between 0 and 22° C., such as around 8° C., such as around 0° C.

In one embodiment, said periodate is used at a concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h, at a temperature of between 0 and 22° C., such as a temperature of 0-8° C., such as a temperature of 0-4° C., such as around 8° C., such as around 0° C.

In one embodiment, said periodate is sodium meta-periodate and said reaction of step a) is performed for a time period of no more than 4 h, such as no more than 3 h, such as no more than 2 h, such as no more than 1 h, such as around 0.5 h at a temperature of between 0 and 22° C., such as a temperature of 0-8° C., such as a temperature of 0-4° C., such as around 8° C., such as around 0° C.

In one embodiment, said periodate is sodium meta-periodate which is used at a concentration of no more than 20 mM, such as no more than 15 mM, such as around 10 mM, and said reaction of step a) is performed at a temperature of between 0 and 22° C., such as a temperature of 0-8° C., such as a temperature of 0-4° C., such as around 8° C., such as around 0° C.

In one embodiment, said periodate is sodium meta-periodate which is used at a concentration around 10 mM, and said reaction of step a) is performed at a temperature of around 8° C. and for a time period of no more than 2 h.

In one embodiment, said periodate is sodium meta-periodate which is used at a concentration of around 10 mM, and said reaction of step a) is performed at a temperature of 0-8° C. and for a time period of no more than 3 h.

In the following paragraphs, specific embodiments of step b) are disclosed. It should be understood that unless defined otherwise, specific embodiments can be combined, in particular specific embodiments of step a) and step b).

In one embodiment, said borohydride is used at a concentration of between 10 and 80 mM.

In one embodiment, said alkali metal borohydride is sodium borohydride.

In some instances, the conditions used for step b) have been found to partly depend on the conditions used for step a). While the amount of borohydride used in step b) is preferably kept as low as possible, the molar ratio of borohydride to periodate is in such instances 0.5-4 to 1. Thus, borohydride may in step b) be used in a molar excess of 4 times the amount of periodate used in step a). In one embodiment, said borohydride is used at a (final) molar concentration of no more than 4 times the (final) concentration of said periodate. For example, borohydride may be used at a concentration of no more than 3 times the concentration of said periodate, such as no more than 2.5 times the concentration of said periodate, such as no more than 2 times the concentration of said periodate, such as no more than 1.5 times the concentration of said periodate, such as at a concentration roughly corresponding to the concentration of said periodate. However, in particular embodiments borohydride is used at a concentration corresponding to half of the periodate concentration, or 0.5 times the periodate concentration. Thus, when periodate is used at a concentration of around 20 mM, borohydride might be used at a concentration of no more than 80 mM, or even at a concentration between 10 and 80 mM, such as at a concentration of between 10 and 50 mM. If periodate is used at a concentration of between 10 and 20 mM, borohydride might be used at a concentration of between 5 and 80 mM, such as for example 50 mM. Similarly, if periodate is used at a concentration of around 10 mM, borohydride might be used at a concentration of no more than 40 mM, such as for example no more than 25 mM. Moreover, in such an embodiment, borohydride may preferably be used at a concentration of between 12 mM and 50 mM. In embodiments where the lysosomal protein is a sulfatase, the concentration of borohydride may influence the degree of preservation of a catalytic amino acid residue at the active site.

In one embodiment, said reaction of step b) is performed for a time period of no more than 1.5 h, such as no more than 1 h, such as no more than 0.75 h, such as around 0.5 h. The reaction duration is preferably around 1 h, or less than 1 h. In some instances, the reaction of step b) has a duration of approximately 0.25 h. In further embodiments, the reaction of step b) may be performed for a time period of from 0.25 h to 2 h. As accounted for above, the duration of the reduction step may affect the biological activity of the lysosomal protein, in particular the catalytic activity of an enzyme such as a sulfatase. A relatively short reaction duration may moreover favorably influence the overall structural integrity of the protein/enzyme. In particular, protein aggregation resulting in high molecular weight forms of lysosomal protein as well as strand-break occurrence may at least partly be related to reaction time.

In one embodiment, said reaction of step b) is performed at a temperature of between 0 and 8° C. Reaction temperature for step b) may at least partly affect biological activity of the reaction product. Thus, it may be advantageous to perform step b) at a temperature of below 8° C. The temperature is preferably around 0° C.

In one embodiment, said alkali metal borohydride is sodium borohydride which is used at a concentration of 0.5-4 times the concentration of said periodate, such as at a concentration of no more than 2.5 times the concentration of said periodate.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed for a time period of no more than 1 h, such as around 0.5 h.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed for a time period of no more than 1 h, such as around 0.5 h, at a temperature of between 0 and 8° C.

In one embodiment, said alkali metal borohydride is used at a concentration of 0.5-4 times the concentration of said periodate, such as at a concentration of no more than 2.5 times the concentration of said periodate, and said reaction of step b) is performed for a time period of no more than 1 h, such as around 0.5 h, at a temperature of between 0 and 8° C.

In one embodiment, said alkali metal borohydride is sodium borohydride, and said reaction of step b) is performed for a time period of no more than 1 h, such as around 0.5 h, at a temperature of between 0 and 8° C.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed at a temperature of between 0 and 8° C.

In one embodiment, said alkali metal borohydride is sodium borohydride which is used at a concentration of 0.5-4 times the concentration of said periodate, such as at a concentration of 2.5 times the concentration of said periodate, and said reaction of step b) is performed at a temperature of around 0° C. for a time period of around 0.5 h.

In one embodiment, said periodate is sodium meta-periodate and said alkali metal borohydride is sodium borohydride.

In one embodiment, each of step a) and step b) is individually performed for a time period of no more than 2 h, such as no more than 1 h, such as around 1 h or around 0.5 h. Optionally, said borohydride is used at a concentration of 0.5-4 times the concentration of said periodate, preferably 0.5-2.5 times the concentration of said periodate. In certain embodiments, said borohydride is used at a concentration of 0.5 times the concentration of periodate, or at a concentration of 2.5 times the concentration of said periodate.

In one embodiment, step a) is performed for a time period of no more than 3 h and step b) is performed for no more than 1 h. Optionally, said borohydride is used at a concentration of no more than 4 times the concentration of said periodate, preferably no more than 2.5 times the concentration of said periodate.

In one embodiment, step a) is performed for a time period of no more than 0.5 h and step b) is performed for no more than 1.5 h. Optionally, said borohydride is used at a concentration of no more than 4 times the concentration of said periodate, preferably no more than 2.5 times the concentration of said periodate.

The person skilled in the art is aware of ways to control the reaction duration of a chemical reaction, such as the reaction duration of each of step a) and b). Thus, in one embodiment, said method aspects further comprises a2) quenching of the reaction resulting from step a). Said quenching for example has a duration of less than 30 minutes, such as less than 15 minutes. In some instances, said quenching is performed immediately after step a). Quenching may for example be performed by addition of ethylene glycol, or another diol, such as for example cis-cyclo-heptane-1,2-diol. Preferably, step b) follows immediately after the quenching. This may minimize the period of exposure for lysosomal protein to reactive aldehyde groups. Reactive aldehydes can promote inactivation and aggregation of the protein.

In one embodiment, said methods further comprises b2) quenching of the reaction resulting from step b). This quenching may for example be conducted by addition of a molecule that contains a ketone or aldehyde group, such as cyclohexanone or acetone, said molecule preferably being soluble in water, or by lowering the pH below 6 of the reaction mixture by addition of acetic acid or another acid. An optional quenching step allows for a precise control of reaction duration for step b).

Thus, in one embodiment, at least one of steps a) and b) is/are performed in the presence of a protective ligand. In particular, step a) may be performed in presence of a protective ligand. A ligand, such as a substrate to said lysosomal protein, may protect the functional epitope or active site of the protein during the steps of oxidation and reduction, and optionally the quenching step(s). The ligand can alternatively be an inhibitor of the protein.

In another embodiment, steps a) and b) of the method are performed while the lysosomal protein is immobilized on a resin. Thus, the lysosomal protein may initially be immobilized on a resin or medium. Then the reactions of steps a) and b), and optionally a2) and b2), may be conducted while the protein is immobilized onto the resin or medium. Suitable resins or mediums are known to the skilled person. For example, anion exchange media or affinity media may be used.

In one embodiment of the method aspects, at least one of steps a) and b) is performed in the presence of a protective ligand, and steps a) and b) are performed while said lysosomal protein is immobilized on a resin.

In one embodiment, steps a) and b) of the method are performed in a continuous process. In particular, steps a), a2), b), and b2) may be performed in a continuous process. The term “continuous process” as used herein should be understood as a process that is continuously operated and wherein reagents are continuously fed to the process unit. By adding the reagents, such as the alkali metal periodate and the alkali metal borohydride, to a stream comprising the lysosomal protein, the reaction can be carried out in a continuous mode. A continuous process can for example be carried out in a multi-pump HPLC system.

The methods as disclosed herein thus provide a modified lysosomal protein having improved properties. It is expected that the conditions for chemical modification of lysosomal protein provides minimal negative impact on structural integrity of the lysosomal protein polypeptide chain, and simultaneously results in substantial absence of natural or unmodified glycan epitopes. Exemplary embodiments of the method are depicted in FIGS. 1B, 1C and 1D.

In a related aspect, there is provided a method of producing a protein, said method comprising:

expressing said protein in mammalian, plant or yeast cells, thereby providing a glycosylated protein, and

modifying epitopes for glycan recognition receptors on said glycosylated protein, thereby reducing the activity of the protein with respect to said glycan recognition receptors.

In one embodiment, said modifying is conducted by sequential reaction with an alkali metal periodate and an alkali metal borohydride. Examples of plant and yeast expression system are known to the skilled person but may include an expression system of species such as Saccharomyces cerevisiae, Pichia Pastoris and Ogataea minuta. An example of a mammalian cell line is a CHO cell line. Other embodiments of said method are disclosed above.

In one aspect, there is provided a modified lysosomal protein obtainable by a method of the above defined method aspects, provided that said protein is not sulfamidase.

In one embodiment of the aspects disclosed herein, said modified lysosomal protein, said lysosomal protein composition or modified lysosomal protein obtainable by any one of the method aspects, is for use in therapy.

In one embodiment of the aspects as disclosed herein, said modified lysosomal protein, said lysosomal protein composition or modified lysosomal protein obtainable by any one of the method aspects, is for use in treatment of a mammal afflicted with a lysosomal storage disease.

In one embodiment of the aspects disclosed herein, said mammalian brain is the brain of a human being. In a related embodiment, said mammal is thus a human. Thus, in one embodiment, said mammalian brain is the brain of a mouse. In a related embodiment, said mammal is thus a mouse.

In one aspect, use of a modified lysosomal protein in the manufacture of a medicament is provided, for crossing the blood brain barrier to treat a lysosomal storage disease, in a mammalian brain, said modification comprises having glycan moieties chemically modified by sequential treatment of the protein with an alkali metal periodate and an alkali metal borohydride, thereby reducing the activity of the lysosomal protein with respect to glycan recognition receptors, such as mannose and mannose-6-phosphate cellular delivery systems, while retaining biological activity of said lysosomal protein, under the proviso that said lysosomal protein is not sulfamidase, β-glucuronidase, tripeptidyl peptidase 1 (TPP1) or alpha L-iduronidase.

In one aspect, use of a modified lysosomal protein in the manufacture of a medicament is provided, for (enhanced) distribution to affected visceral organs and/or peripheral tissue in a mammal to treat a lysosomal storage disease in said affected visceral organs and/or peripheral tissue, said modification comprises having glycan moieties chemically modified by sequential treatment of the protein with an alkali metal periodate and an alkali metal borohydride, thereby reducing the activity of the modified lysosomal protein with respect to glycan recognition receptors, such as mannose and mannose-6-phosphate cellular delivery systems, while retaining biological activity of said lysosomal protein. In certain embodiments, said lysosomal protein is not sulfamidase, 6-glucuronidase, tripeptidyl peptidase 1 (TPP1) or alpha L-iduronidase.

In one embodiment of the aspects as disclosed herein, said lysosomal storage disease is selected from mannosidosis beta A; lysosomal; leukoencephalopathy; cystic; without megalencephaly (LCWM); mannosidosis, alpha B; lysosomal (MANSA); ceroid lipofuscinosis, neuronal 2 (CLN2); spinocerebellar ataxia; autosomal recessive 7 (SCAR7); ceroid lipofuscinosis, neuronal; 5 (CLNS); Gaucher disease (GD); fucosidosis (FUCA1D); myeloperoxidase deficiency (MPOD); Fabry disease (FD); GM2-gangliosidosis 1 (GM2G1); ceroid lipofuscinosis, neuronal, 10 (CLN10); combined saposin deficiency (CSAPD); Leukodystrophy metachromatic due to saposin-B deficiency (MLD-SAPB); Gaucher disease, atypical, due to saposin C deficiency (AGD); Krabbe disease, atypical, due to saposin A deficiency (AKRD); defects in PSAP saposin-D region are found in a variant of Tay-Sachs disease (GM2-gangliosidosis); GM2-gangliosidosis 2 (GM2G2); mucopolysaccharidosis 7 (MPS7); glycogen storage disease 2 (GSD2); galactosialidosis (GSL); spondyloenchondrodysplasia with immune dysregulation (SPENCDI); leukodystrophy metachromatic (MLD); mucopolysaccharidosis 3D (MPS3D); mucopolysaccharidosis 6 (MPS6); GM1-gangliosidosis 1 (GM1G1); GM1-gangliosidosis 2 (GM1G2); GM1-gangliosidosis 3 (GM1G3); mucopolysaccharidosis 4B (MPS4B); Schindler disease (SCHIND); Kanzaki disease (KANZD); Niemann-Pick disease A (NPDA); Niemann-Pick disease B (NPDB); GM2-gangliosidosis AB (GM2GAB); aspartylglucosaminuria (AGU); mucopolysaccharidosis 2 (MPS2); mucopolysaccharidosis 4A (MPS4A); mucopolysaccharidosis 1H (MPS1H); mucopolysaccharidosis 1H/S (MPS1H/S); mucopolysaccharidosis 1S (MPS1S); Wolman disease (WOD); cholesteryl ester storage disease (CESD); pycnodysostosis (PKND); ceroid lipofuscinosis, neuronal, 1 (CLN1); mucopolysaccharidosis 3A (MPS3A); Papillon-Lefevre syndrome (PLS); Haim-Munk syndrome (HMS); periodontititis, aggressive, 1 (API); mucopolysaccharidosis 3B (MPS3B); leukodystrophy, globoid cell (GLD); Niemann-Pick disease C2 (NPC2); mucopolysaccharidosis 9 (MPS9); Farber lipogranulomatosis (FL); spinal muscular atrophy with progressive myoclonic epilepsy (SMAPME); hypercholesterolemia, autosomal dominant, 3 (HCHOLA3); sialidosis (SIALIDOSIS); autoimmune disease 6 (A156); ceroid lipofuscinosis, neuronal, 13 (CLN13), and multiple sulfatase deficiency (MSD).

In one embodiment, said modified lysosomal protein, lysosomal protein composition, or modified lysosomal protein obtainable by the method aspect for use in therapy reduces lysosomal storage in the brain of said mammal. In particular, said storage is reduced by at least 30% in e.g. an animal model, such as at least 35%, at least 40%, at least 50%, or at least 60%.

In one aspect there is provided a method of treating a mammal afflicted with a lysosomal storage disease, comprising administering to the mammal a therapeutically effective amount of a modified lysosomal protein, said modified lysosomal protein being selected from:

a) a modified lysosomal protein as described in, or obtainable from, aspects and embodiments disclosed herein, and

b) a lysosomal protein composition as described in aspects and embodiments herein.

In one embodiment thereof, said treatment results in clearance of about at least 50% lysosomal storage from the brain of a mammal after administration of 10 doses of modified lysosomal protein over a time period of 70 days.

The invention will be further illustrated by the following non-limiting examples.

### BRIEF DESCRIPTION OF THE DRAWINGS

FIG. 1 is a picture outlining the differences between the methods for chemical modification developed by the inventors, disclosed in Example 3, and the known method, disclosed in WO 2008/109677.

FIG. 2A shows a SDS-PAGE gel of sulfamidase (lane 1), sulfamidase modified according to the known method (lane 2), iduronate 2-sulfatase (lane 3) and iduronate 2-sulfatase modified according to the known method (lane 4), alpha-L-iduronidase (lane 5) and alpha-L-iduronidase modified according to the known method (lane 6). Four protein bands, denoted 1-4, generated by the glycan modification procedure of sulfamidase were identified (lane 2).

FIG. 2B shows SDS-PAGE gels of sulfamidase, iduronate 2-sulfatase and alpha-L-iduronidase modified according to the known method (lane 1, 3 and 5) and modified according to the new methods disclosed herein (lane 2, 4, 6, 7 and 8).

FIG. 3A shows a SEC chromatogram of sulfamidase modified according to the known method.

FIG. 3B shows a SEC chromatogram of sulfamidase modified according to new method 1 as disclosed herein. Marked by an arrow is the peak of multimeric forms of modified sulfamidase.

FIG. 4A shows scattering intensity measured by dynamic light scattering of sulfamidase modified according to the known method.

FIG. 4B shows scattering intensity measured by dynamic light scattering of sulfamidase modified according to new method 1 as described herein.

FIG. 5 is a diagram visualizing the receptor mediated endocytosis in MEF-1 cells of unmodified recombinant sulfamidase, sulfamidase modified according to the known method and sulfamidase modified according to new method 1 and 4 as described herein.

FIG. 6A shows the results from in vivo treatment of MPS IIIA deficient mice. The diagram shows clearance of heparan sulfate storage in the brain of mice after i.v. dosing every other day (13 doses) of sulfamidase modified according to new method 1 at 30 mg/kg.

FIG. 6B shows the results from in vivo treatment of MPS IIIA deficient mice. The diagram shows clearance of heparan sulfate storage in the liver of mice after i.v. dosing every other day (13 doses) of sulfamidase modified according to new method 1 at 30 mg/kg.

FIG. 6C shows the results from in vivo treatment of MPS IIIA deficient mice. The diagram shows clearance of heparan sulfate storage in the brain of mice after i.v. dosing once weekly (10 doses) of sulfamidase modified according to new method 1 at 30 mg/kg and 10 mg/kg, respectively.

FIG. 7 is a schematic drawing of the three archetypal N-glycan structures generally present in proteins of mammalian origin and the typical N-glycan present in yeast proteins. The left glycan represents the oligomannose type, the second from the left the complex type, and the second from the right the hybrid type. The one on the far right is the polymannose type of yeast proteins. In the Figure the following compounds are depicted: black filled diamonds correspond to N-acetylneuraminic acid; black filled circles correspond to mannose; squares correspond to N-acetylglucosamine; black filled triangle corresponds to fucose; circle corresponds to galactose. Sugar moieties marked with an asterisk can be modified by periodate/borohydride treatment disclosed herein.

FIG. 8A is a schematic drawing illustrating predicted bond breaks on mannose after chemical modification.

FIG. 8B is a schematic drawing illustrating a model of a Man-6 glycan. The sugar moieties suscpetible to bond breaks upon oxidation with periodate are indicated. Grey circles correspond to mannose, black squares correspond to N-acetylglucosamine, T13 corresponds to the tryptic peptide NITR of sulfamidase (SEQ ID NO:44) with the N-glycosylation site N(131) included.

FIG. 9 represents mass spectra of doubly charged ions corresponding to tryptic peptide T13 of sulfamidase (SEQ ID NO:44) with Man-6 glycan attached to N(131) (T13+Man-6 glycan), prior to (A) and after chemical modification (B-D) according to previously known method (S.=single bond breaks; D.=double bond breaks; e.g. D.x3=3 double bond breaks).

FIG. 10A is a diagram visualizing the extent of bond breaking of the tryptic peptide T13+Man-6 glycan after chemical modification of sulfamidase (SEQ ID NO:44) according to the previously known method (black bar), new method 1 (black dots), new method 3 (white), and new method 4 (cross-checkered).

FIG. 10B is a diagram visualizing the relative abundance of single bond breaks in the tryptic peptide T13+Man-6 glycan after chemical modification of sulfamidase (SEQ ID NO:44) according to the previously known method (black bar), new method 1 (black dots), new method 3 (white), and new method 4 (cross-checkered).

FIG. 11 is a diagram showing the activity of iduronate 2-sulfatase as well as iduronate 2-sulfatase modified according to new method 10 and 11.

### EXAMPLES

The Examples which follow disclose the development of modified lysosomal proteins, exemplified by sulfamidase, alpha-L-iduronidase and iduronate 2-sulfatase.

Materials and Methods

The recombinant alpha-L-iduronidase used in the Examples below was the medicinal product Aldurazyme® whereas the recombinant iduronate 2-sulfatase was the medicinal product Elaprase®. Both were purchased from a pharmacy (Apoteket farmaci, Sweden), stored according to the manufacturer's specifications and treated under sterile conditions.

The sulfamidase was produced by cloning and transient expression in HEK293 cells using the pcDNA3.1(+) vector and in CHO with the Quattromed Cell Factory (QMCF) episomal expression system (Icosagen AS) using the pQMCF1 vector. Sulfamidase was captured from medium by anion exchange chromatography (AlEX) on a Q sepharose column (GE Healthcare) equilibrated with 20 mM Tris, 1 mM EDTA, pH 8.0 and eluted by a NaCl gradient. Captured sulfamidase was further purified by 4-Mercapto-Ethyl-Pyridine (MEP) chromatography; sulfamidase containing fractions were loaded on a MEP HyperCel chromatography column and subsequently eluted by isocratic elution in 50 mM NaAc, 0.1 M NaCl, 1 mM EDTA, 1 mM DTT, pH 4.6. Final polishing was achieved by cation exchange chromatography (CIEX) on a SP Sepharose FF (GE Healthcare) column equilibrated in 25 mM NaAc, 2 mM DTT, pH 4.5. A NaCl gradient was used for elution.

### Example 1

### Chemical Modification of the Lysosomal Proteins Sulfamidase, Alpha-L-Iduronidase and Iduronate 2-Sulfatase According to Previously Known Method

Chemical Modification According to WO 2008/109677:

In order to modify glycan moieties, above mentioned lysosomal proteins were initially incubated with 20 mM sodium meta-periodate at 0° C. for 6.5 h in 20 mM sodium phosphate, 137 mM NaCl (pH 6.0). Glycan oxidation was quenched by addition of ethylene glycol to a final concentration of 192 mM. Quenching was allowed to proceed for 15 min at 0° C. before performing dialysis against 20 mM sodium phosphate, 137 mM NaCl (pH 6.0) over night at 4° C. Following dialysis, reduction was performed by addition of sodium borohydride to the reaction mixture at a final concentration of 100 mM. The reduction reaction was allowed to proceed over night. Finally, enzyme preparations were dialyzed against 20 mM sodium phosphate, 137 mM NaCl (pH 6.0). All incubations were performed in the dark.

### Example 2

### Analyses of the Lysosomal Proteins Sulfamidase, Alpha-L-Iduronidase and Iduronate 2-Sulfatase Modified According to Known Method

Material and Methods

SDS-PAGE Analysis:

The lysosomal enzymes modified according to the known method as described in Example 1 was subjected to SDS-PAGE analysis with protein loaded on NuPAGE 4-12% Bis-Tris gels. Seeblue 2 plus marker was used for molecular weight calibration and the gels were stained with Instant Blue (C.B.S Scientific).

Glycan Analysis by LC/MS of Tryptic Fragments:

The glycosylation patterns were determined by LC/MS of tryptic fragments of the three lysosomal proteins of Example 1. Prior to glycopeptide analysis, proteins were reduced, alkylated and digested with trypsin. Reduction of the protein was done by incubation in 5 μl DTT 10 mM in 50 mM NH₄HCO₃ at 60° C. for 1 h (70° C. for alpha-L-iduronidase). Subsequent alkylation with 5 μl iodoacetamide 55 mM in 50 mM NH₄HCO₃ was performed at room temperature (RT) and in darkness for 45 min. Lastly, the tryptic digestion was performed by addition of 30 μl of 50 mM NH₄HCO₃, 5 mM CaCl₂, pH 8, and 0.2 μg/μl trypsin in 50 mM acetic acid (protease:protein ratio 1:20 (w/w)). Digestion was allowed to take place over night at 37° C.

Possible glycosylation variants of the tryptic peptide fragments were investigated by glycopeptide analysis. This was performed by liquid chromatography followed by mass spectrometry (LC-MS) on an Agilent 1200 HPLC system coupled to an Agilent 6510 Quadrupole time-of-flight mass spectrometer (Q-TOF-MS). Both systems were controlled by MassHunter Workstation. LC separation was performed by the use of a Waters XSELECT CSH 130 C18 column (150×2.1 mm), the column temperature was set to 40° C. Mobile phase A consisted of 5% acetonitrile, 0.1% propionic acid, and 0.02% TFA, and mobile phase B consisted of 95% acetonitrile, 0.1% propionic acid, and 0.02% TFA. A gradient of from 0% to 10% B for 10 minutes, then from 10% to 70% B for another 25 min was used at a flow rate of 0.2 mL/min. The injection volume was 10 μl. The Q-TOF was operated in positive-electrospray ion mode. During the course of data acquisition, the fragmentor voltage, skimmer voltage, and octopole RF were set to 90, 65, and 650 V, respectively. Mass range was between 300 and 2800 m/z.

The following analyses were conducted only for sulfamidase preparations.

Dynamic Light Scattering (DLS) Analysis of Sulfamidase:

The modified sulfamidase was degassed by centrifugation at 12000 rpm for 3 min at room temperature (RT). DLS experiments were performed on a DynaPro Titan instrument (Wyatt Technology Corp) using 25% laser power with 3 replicates of 75 μL each.

Analysis by Size Exclusion Chromatography (SEC) of Sulfamidase:

The modified enzyme was analyzed by analytical size exclusion chromatography, performed on a AKTAmicro system (GE Healthcare). A Superdex 200 PC 3.2/30 column with a flow rate of 40 μL/min of formulation buffer was used. The sample volume was 10 μL and contained 10 μg enzyme.

In-Gel Digestion and MALDI-TOF MS Analysis of Sulfamidase:

The SDS-PAGE analysis revealed some extra bands, which were excised, destained and processed by in-gel digestion with trypsin. Digestion was performed over night at 37° C. The supernatant was transferred to a new tube and extracted with 60% acetonitrile, 0.1% TFA (3×20 min) at RT. The resulting supernatants were evaporated in a Speed Vac to near dryness. The concentrated solution was mixed 1:1 with alpha-cyano-4-hydroxycinnamic acid solution (10 mg/mL) and 0.6 μL was applied on a MALDI plate. Molecular masses of the tryptic peptide fragments were determined using a Sciex 5800 matrix-assisted laser desorption/ionization-time-of-flight mass spectrometer (MALDI-TOF/TOF MS). The analyses were performed in positive ion reflectron mode with a laser energy of 3550 and 400 shots.

Preservation of Sulfamidase Active Site:

Any effect of the chemical modification on the active site of sulfamidase was investigated by the use of LC-MS and LC-MS/MS analyses. The samples were prepared according to the LC-MS method described under section Glycosylation analysis. The resulting tryptic peptides containing cysteine 50 variants (cysteine50 (alkylated), oxidized cysteine 50, FGly50 and Ser50) were all semiquantified using peak area calculations from reconstructed ion chromatograms. The identity of the peptides was confirmed by MSMS sequencing. The MSMS parameters were as follows: the collision energies were set to 10, 15, and 20V, scan range 100-1800 m/z, and scan speed 1 scan/sec.

Results

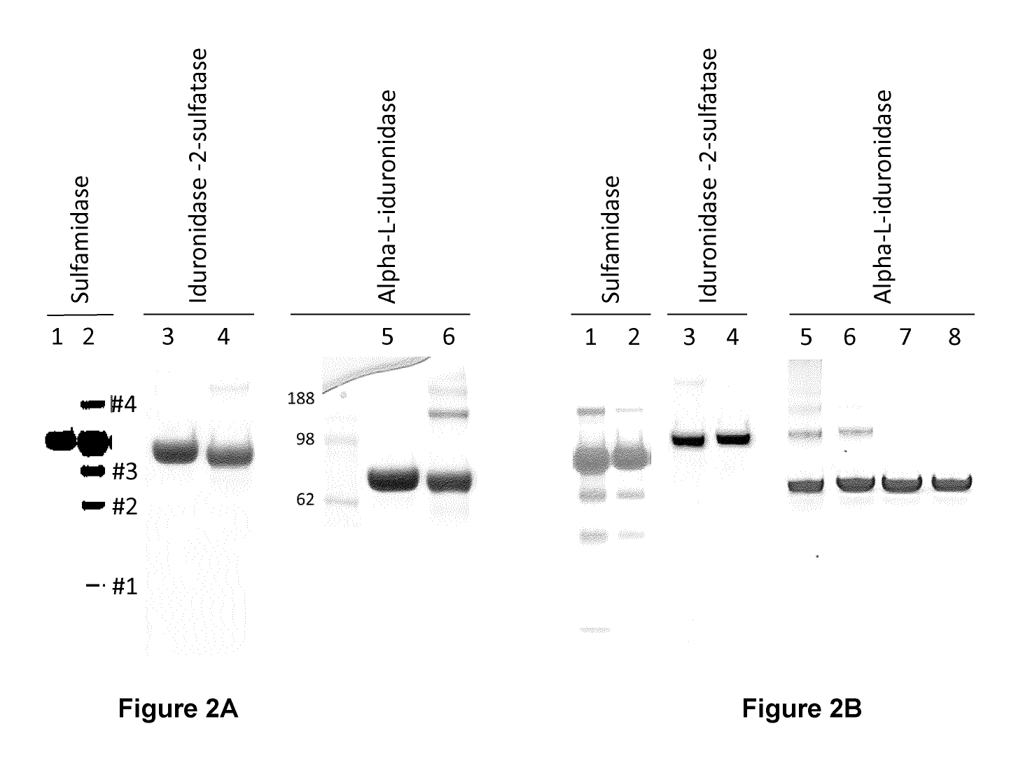

As apparent by SDS-PAGE analysis, several major peptides of sizes distinct from that of the full length proteins were formed as a result of the chemical modification according to the known method (FIG. 2A). Peptide bands of lower molecular weight, representing peptide cleavage products are apparent for all three lysosomal proteins, although it was most prominent for sulfamidase. By MALDI-TOF MS analysis, four gel bands #1-4 observed on SDS-PAGE (FIG. 2A, lane 2) could be identified as fragments of sulfamidase generated by strand breaks during the chemical modification. The gel bands #1 and #2 were determined as two C-terminal truncations with molecular masses of 6 kDa and 30 kDa, gel band #3 as one 41 kDa N-terminal truncation.

It was also found that the chemical modification according the known method introduces oxidation on several methionine residues on sulfamidase, in particular on methionine 184 and methionine 443, which were almost completely oxidized. Methionine 226 (found in a tryptic peptide corresponding to amino acid residues 226-238) was oxidized to a much lower degree, but this oxidation appeared to give rise to a more unstable protein than unmodified sulfamidase as such, generating the 41 kDa N-terminal truncation. Thus, oxidation of methionine 226 and strand breaks seemed to be correlated, as observed in the MS analysis.

Notably, bands of higher molecular weight were apparent for all three lysosomal proteins indicating covalent multimerisation as a consequence of chemical modification according to the known method. For sulfamidase, the predominant band could be identified as a dimer of a molecular weight of 111 kDa (FIG. 2A, lane 2, band #4). Most severe multimerisation was seen for alpha-L-iduronidase (FIG. 2A, lane 6).

Thus, it was found that chemical modification of sulfamidase in accordance to the known method (WO 2008/109677) not only modifies glycans but also generates polypeptide strand breaks, covalent multimerisation and oxidation of amino acid residues crucial for structural integrity of the enzyme.

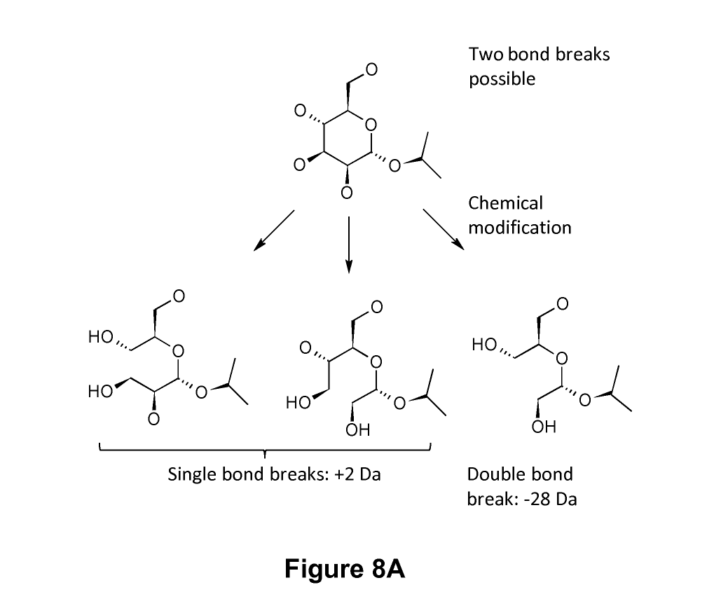

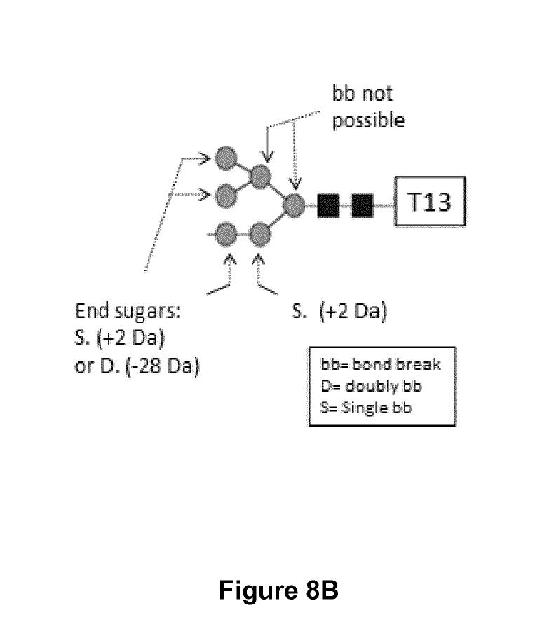

SDS-PAGE analysis also clearly showed a common lowering of the position of the main monomeric band for all three lysosomal proteins when compared to unmodified protein (FIG. 2A lane 1 vs lane 2; lane 3 vs lane 4; lane 5 vs lane 6). This suggests a loss of molecular weight of approximately 500-1500 Da and is expected for a chemical reaction where glycan moieties predominantly are modified by double bond breaks (FIG. 8).

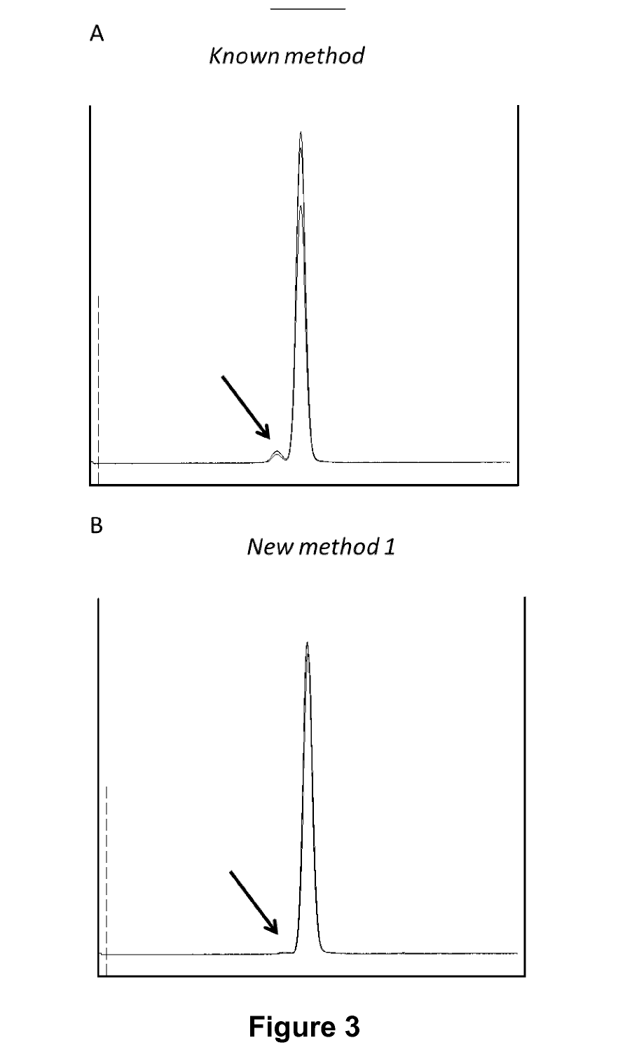

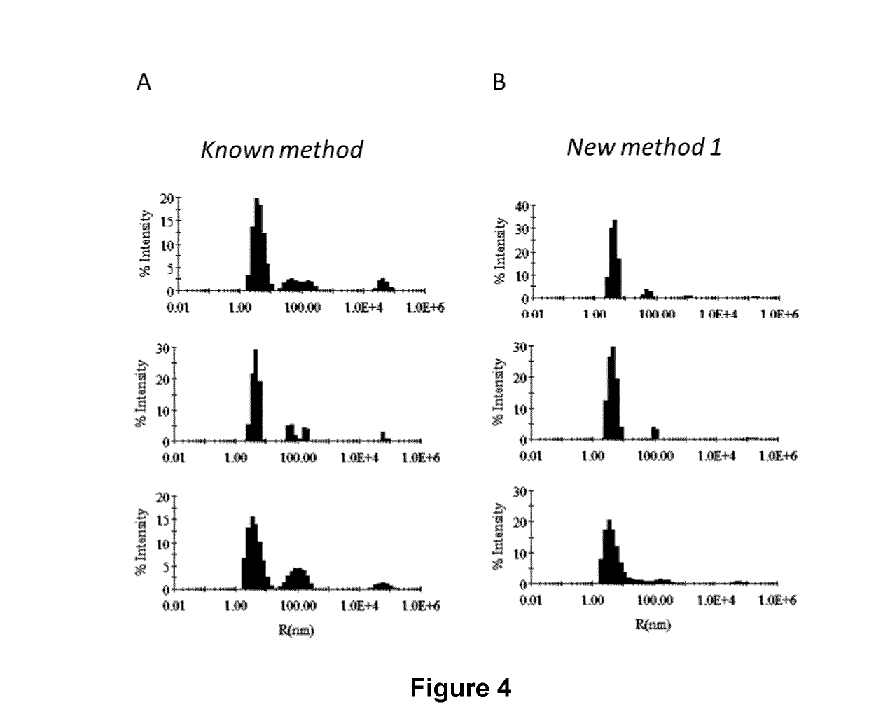

Further analysis of sulfamidase by SEC revealed that the chemical modification procedure according to the known method promoted aggregation of sulfamidase, as demonstrated as a pre-peak in the chromatogram of FIG. 3A. The peak height of the pre-peak in the chromatogram was found to be approximately 3% of the height of the main peak. The DLS analysis moreover revealed that the same material contained 15-20% of protein of the total protein content in high molecular weight forms (i.e. above 10¹⁰ kDa) (FIG. 4A).

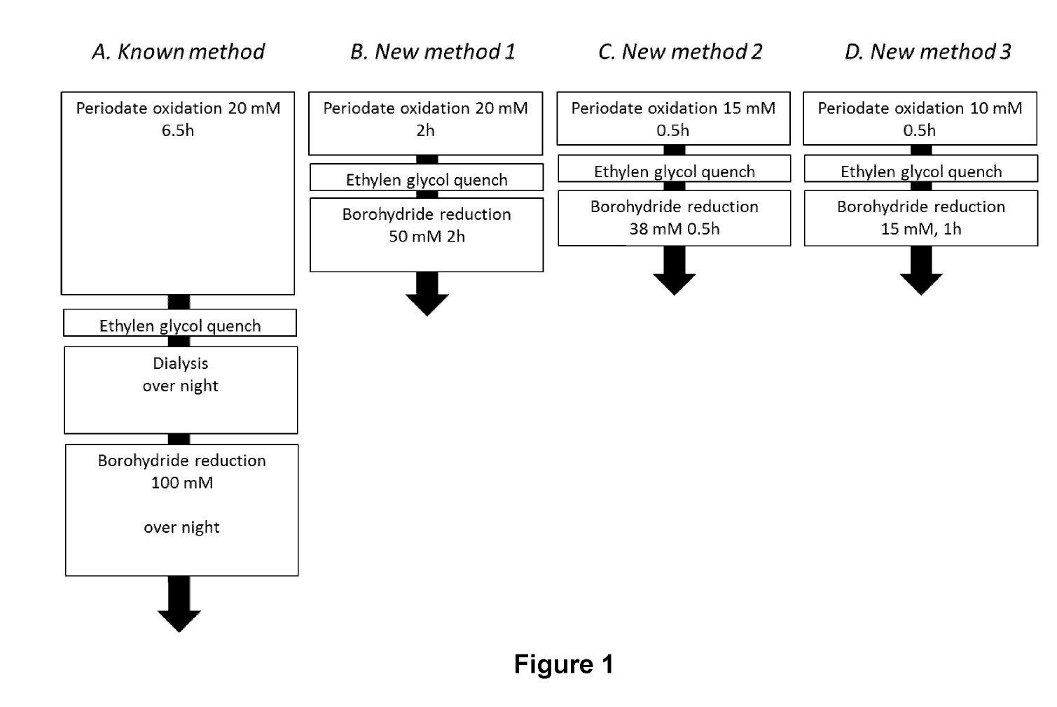

Moreover, by the use of LC-MSMS, the reduction step (FIG. 1A) was found to reduce the FGly residue at the active site position 50 of sulfamidase (SEQ ID NO:44) to Ser. Ser in this position is not compatible with efficient catalysis (Recksiek et al, J Biol Chem 273(11):6096-103 (1998)). The relative amount Ser produced from FGly was estimated based on peak area measurements of the doubly charged ions in the mass spectrum, corresponding to the two tryptic peptide fragments containing FGly50 and Ser50. The peak areas were based on MS response without correction for ionization efficiency. Table 2 below shows that the conversion of FGly to Ser is approximately 56% according to the known method (see also Example 4, Table 3).

TABLE 2
Conversion of FGly to Ser at active site

| Chemical modification of sulfamidase | Ser formation (%) | FGly/Ser ratio |
|---|---|---|
| None | 0 | |
| WO 2008/109677 | 56.0 ± 0.3 (n = 3) | ca 0.79 |

Thus, the known chemical modification procedure, in addition to the modifications mentioned above, causes reduction of an amino acid residue crucial for catalytic activity of sulfamidase. The FGly residue is present in all sulfatases and is crucial for enzymatic activity.

Glycan analysis by LC/MS of tryptic fragments, confirmed that no natural glycans were present in the lysosomal proteins studied after chemical modification, indicative of complete modification of the glycans.

### Example 3

### New Methods for Chemical Modification of the Lysosomal Enzymes Sulfamidase, Alpha-L-Iduronidase and Iduronate 2-Sulfatase

Chemical Modification According to New Method 1:

The above mentioned lysosomal proteins were initially incubated at 20 mM sodium meta-periodate at 0° C. in the dark for 120 min in phosphate buffers having a pH of 6.0. Glycan oxidation was quenched by addition of ethylene glycol to a final concentration of 192 mM. Quenching was allowed to proceed for 15 min at 6° C. before sodium borohydride was added to the reaction mixtures to a final concentration of 50 mM. After incubation at 0° C. for 120 min in the dark, the resulting protein preparations were ultrafiltrated against 20 mM sodium phosphate, 100 mM NaCl, pH 6.0. The new method 1 for chemical modification is depicted in FIG. 1B.

Chemical Modification According to New Method 2:

The above mentioned lysosomal proteins were initially incubated at 15 mM sodium meta-periodate at 0° C. for 0.5 h in 20 mM sodium phosphate, 137 mM NaCl (pH 6.0). Glycan oxidations were quenched by addition of ethylene glycol to a final concentration of 96 mM. Quenching was allowed to proceed for 15 min at 0° C. Thereafter sodium borohydride was added to the reaction mixtures to a final concentration of 38 mM and the resulting mixtures were held at 0° C. for 0.5 h. Finally, the enzyme preparations were ultrafiltrated against 20 mM sodium phosphate, 137 mM NaCl (pH 6.0). All incubations were performed in the dark. The new method 2 for chemical modification is depicted in FIG. 1C.

Chemical Modification According to New Method 3:

The above mentioned lysosomal proteins were initially incubated at 10 mM sodium meta-periodate at 0° C. for 0.5 h in 20 mM sodium phosphate, 137 mM NaCl (pH 6.0). Glycan oxidations were quenched by addition of ethylene glycol to a final concentration of 96 mM. Quenching was allowed to proceed for 15 min at 0° C. Thereafter sodium borohydride was added to the reaction mixtures to a final concentration of 15 mM and the resulting mixtures were held at 0° C. for 1 h. Finally, the enzyme preparations were ultrafiltrated against 20 mM sodium phosphate, 137 mM NaCl (pH 6.0). All incubations were performed in the dark. The new method 3 for chemical modification is depicted in FIG. 1D.

Here follow examples of new methods evaluated and exemplified with one specific lysosomal enzyme.

New Method 4:

Exemplified for sulfamidase. Performed as New method 1 with the exception that the concentration of sodium borohydride in the reduction step was 10 mM.

New Method 5:

Exemplified for sulfamidase. Sulfamidase was oxidized by incubation with 10 mM sodium meta-periodate at 0° C. in the dark for 180 min in acetate buffer having an initial pH of between 4.5 to 6. Glycan oxidation was quenched by addition of ethylene glycol to a final concentration of 192 mM. Quenching was allowed to proceed for 15 min at 6° C. before sodium borohydride was added to the reaction mixture to a final concentration of 25 mM. After incubation at 0° C. for 60 min in the dark, the resulting sulfamidase preparation was ultrafiltrated against 10 mM sodium phosphate, 100 mM NaCl, pH 7.4.

New Method 6:

Exemplified for sulfamidase. Sulfamidase was oxidized by incubation with 10 mM sodium meta-periodate at 8° C. in the dark for 60 min in acetate buffer having an intial pH of 4.5. Glycan oxidation was quenched by addition of ethylene glycol to a final concentration of 192 mM. Quenching was allowed to proceed for 15 min at 6° C. before sodium borohydride was added to the reaction mixture to a final concentration of 25 mM. After incubation at 0° C. for 60 min in the dark, the resulting sulfamidase preparation was ultrafiltrated against 10 mM sodium phosphate, 100 mM NaCl, pH 7.4.

New Method 7:

Exemplified for sulfamidase. Sulfamidase was oxidized by incubation with 10 mM sodium meta-periodate at 8° C. in the dark for 60 min in acetate buffer having an intial pH of 4.5. Glycan oxidation was quenched by addition of ethylene glycol to a final concentration of 192 mM. Quenching was allowed to proceed for 15 min at 6° C. before sodium borohydride was added to the reaction mixture to a final concentration of 25 mM. After incubation at 0° C. for 30 min in the dark, the resulting sulfamidase preparation was ultrafiltrated against 10 mM sodium phosphate, 100 mM NaCl, pH 7.4.

New Method 8:

Exemplified for alpha-L-iduronidase. Alpha-L-iduronidase was initially incubated at 15 mM sodium meta-periodate at 0° C. for 20 min in 20 mM sodium phosphate, 137 mM NaCl (pH 6.0). Glycan oxidation was quenched by addition of ethylene glycol to a final concentration of 96 mM. Quenching was allowed to proceed for 15 min at 0° C. Thereafter sodium borohydride was added to the reaction mixture to a final concentration of 37 mM and the resulting mixture was held at 0° C. for 1 h. Finally, the enzyme preparation was ultrafiltrated against 20 mM sodium phosphate, 137 mM NaCl (pH 6.0). All incubations were performed in the dark.

New Method 9:

Exemplified for alpha-L-iduronidase. Reaction conditions were as described for new method 8, with the single exception that periodate oxidation was performed in the presence of 100 μM 4-methylumbeliferone iduronide, functioning as a protecting ligand during the oxidation step.

Results

As already accounted for elsewhere herein, sodium meta-periodate is an oxidant that converts cis-glycol groups of carbohydrates to aldehyde groups, whereas borohydride is a reducing agent that reduces the aldehydes to more inert alcohols. The carbohydrate structure is thus irreversibly destroyed.

In order to provide an improved method for chemical modification of glycans, in particular a procedure that provides a modified lysosomal protein with improved properties, a significant number of reaction conditions were evaluated. It could be concluded that both oxidation by sodium meta-periodate and reduction by sodium borohydride introduced polypeptide modifications and aggregation; properties that negatively impact on catalytic activity and immunogenic propensity.

Conditions were discovered for an improved chemical modification procedure (Exemplified by new method 1-9). Surprisingly, the structural integrity and activity of the lysosomal proteins could be retained given that the step of sodium borohydride reduction was following directly after quenching of the sodium meta-periodate oxidation and reactant concentrations and time for reactions were kept balanced and significantly lower/shorter as compared to the known method. The new methods omit buffer change and long exposure of the lysosomal protein to reactive aldehyde intermediates. Examples of the new chemical modification procedures are depicted in FIG. 1B-1D.

### Example 4

### Analyses of Sulfamidase, Alpha-L-Iduronidase and Iduronate 2-Sulfatase Modified According to New Methods

The experimental methods described in Example 2 were used to analyze lysosomal proteins modified according to the new methods.

Results

Peptide bands of lower molecular weight, representing peptide cleavage products were apparent also for material modified according to the new methods but at a significantly lower extent (FIG. 2B, lane 1 vs lane 2; lane 3 vs lane 4; lane 5 vs lane 6, 7 and 8). As for alpha-L-iduronidase modified according to new methods 8, only the monomeric band was apparent (FIG. 2B lane 7). Importantly, the use of a ligand protecting the active site (new method 9, FIG. 2B lane 8) was compatible with the procedure and resulted in modified alpha-L-iduronidase that by SDS-PAGE analysis was indistinguishable from that where the ligand was omitted (new method 8).

In conclusion, process related impurities, limiting the quality and safety of a medicament produced by the modification methods, are significantly reduced by the new methods as compared to the previously known method.

Glycan analysis of selected tryptic peptide fragment showed that no, or in some cases less than 5%, naturally occurring glycan structures were present after chemical modification, indicative of complete or close to complete modification of the glycans.

Further analysis of sulfamidase by SEC showed that the sulfamidase modified according to the new method 1 contained less aggregates compared to the sulfamidase modified by the known method. This is demonstrated in the chromatograms of FIG. 3, where the high molecular weight form is present in the chromatogram as a pre-peak. The peak height of the pre-peak in FIG. 3B is 0.5%, relative the main peak height, thus representing a decrease compared to peak height (3%) in FIG. 3A. This is also the case for sulfamidase modified by new method 5 and 6 (data not shown). The DLS analysis (FIG. 4B) confirmed the results from SEC analysis: the sulfamidase produced according to the new method contained 5% protein in high molecular weight forms (above 10¹⁰ kDa). It could thus be concluded that formation of aggregated sulfamidase is limited by the new method.

Sulfamidase was further studied by evaluation of degree of active site preservation: The reduction of FGly to Ser in position 50 at the active site of sulfamidase was determined by LC-MS/MS and the tryptic peptides containing FGly and Ser were positively identified. The relative amount of the peptide fragments was analyzed with LC-MS by measuring the peak areas from reconstructed ion chromatograms of the doubly charged ions (without correction for ionization efficiency). The samples generated by four of the new methods described in Example 3 for the chemical modification were prepared and analyzed in duplicates or triplicates (Table 3)

TABLE 3
Conversion of FGly to Ser at active site

| Chemical modification of | | |
|---|---|---|
| sulfamidase | Ser formation (%) | FGly/Ser ratio |
| None | 0 | |
| New method 1 | 45.4 ± 0.9 (n = 3) | 1.2 |
| New method 4 | 11.5 ± 1 (n = 3) | 7.7 |
| New method 5 | 44.1 ± 2 (n = 2) | 1.2 |
| New method 6 | 34.4 ± 2 (n = 2) | 1.9 |

Loss of active site FGly is limited considerably by the new methods. The four new methods of modifying glycans on sulfamidase significantly decreased the amount of Ser formation, from 56% using the procedure described in WO 2008/109677 (see Table 2, Example 2), to 45%, 44%, and 34% (new method 1, 5, and 6, respectively, Table 3). The Ser formation of the new method 4 was about 11%, thus indicating that the conversion of FGly to Ser was highly dependent on sodium borohydride concentration.

### Example 5

### Receptor Mediated Endocytosis of Chemically Modified Lysosomal Proteins In Vitro

Material and Methods

Sulfamidase was prepared as described and modified according to the known method and new methods 1 and 4 (Example 1 and 3). Endocytosis was evaluated in MEF-1 fibroblasts expressing M6P receptors. The MEF-1 cells were incubated for 24 h in DMEM medium supplemented with 75 nM of sulfamidase. The cells were washed twice in DMEM and once in 0.9% NaCl prior to cell lysis using 1% Triton X100. Lysate sulfamidase activity and total protein content were determined and lysate specific activity was calculated. Activity was monitored by fluorescence intensity at 460 nm using 0.25 mM 4-methylumbelliferyl-alpha-D-N-sulphoglucosaminide as substrate in 14.5 mM diethylbarbituric acid, 14.5 mM sodium acetate, 0.34% (w/v) NaCl, and 0.1% BSA. Total protein concentration was determined using the BCA kit (Pierce) with BSA as standard. Data are presented as mean+SD (n=4).

Results

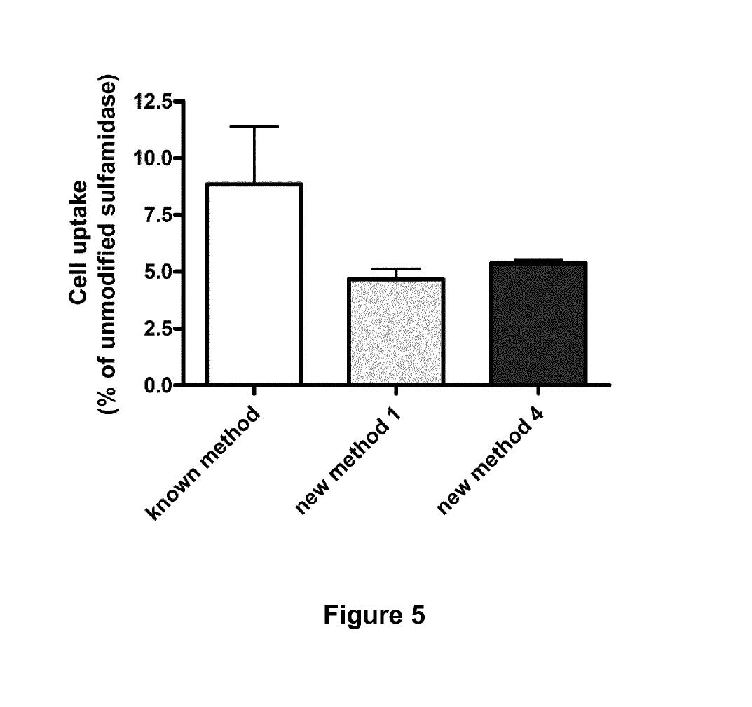

Sulfamidase activity could be detected in cell homogenate for all preparations evaluated in the endocytosis assay. Modified sulfamidase prepared by the known method as well as the new methods 1 and 4 showed specific activities in cell homogenate below 10% of that obtained with unmodified recombinant sulfamidase (FIG. 5). The activity retained in cells first loaded with and then grown in the absence of sulfamidase for 2 days were comparable for all preparations showing that chemical modification do not negatively impact on lysosomal stability.

It can therefore be concluded that chemical modification render sulfamidase less prone to cellular uptake which is a consequence of removal of epitopes for glycan recognition receptors as M6PR. On a macroscopic level, this loss of molecular interactions translates into a reduced clearance from plasma when administrated intravenously. The reduced clearance of the protein could allow for less frequent dosing for the patients. Similar results were obtained with modified alpha-L-iduronidase and iduronate 2-sulfatase (Data not shown).

### Example 6

### In Vivo Plasma/Serum Clearance of Lysosomal Proteins Sulfamidase, Alpha-L-Iduronidase and Iduronate 2-Sulfatase Modified According to New Methods

Material and Methods

In Life Phase:

Plasma/Serum clearance (CL) was investigated for the unmodified and modified lysosomal proteins sulfamidase, alpha-L-iduronidase and iduronate 2-sulfatase in mice (C57BL/6J). Mice were given an intravenous single dose administration in the tail vein. Blood samples were taken at different time points up to 24 h post dose (3 mice per time point) and plasma/serum was prepared. The plasma/serum levels of lysosomal enzymes were analyzed by electrochemiluminescence (ECL) immunoassay. Plasma/serum clearance was calculated using WinNonlin software version 6.3 (Non-compartmental analysis, Phoenix, Pharsight Corp., USA). For sulfamidase and sulfamidase modified according to new method 1 the dose was 10 mg/kg formulated at 2 mg/mL and administered at 5 mL/kg. For iduronate 2-sulfatase and iduronate 2-sulfatase modified according to new method 2 the dose was 1 mg/kg formulated at 0.2 mg/mL and administered at 5 mL/kg. For alpha-L-iduronidase and alpha-L-iduronidase modified according to new method 3 the dose was 3 mg/kg formulated at 0.6 mg/mL and administered at 5 mL/kg.

Quantification of Sulfamidase and Modified Sulfamidase by ECL:

Sulfamidase and modified sulfamidase in plasma PK samples were determined by ECL immunoassay using the Meso Scale Discovery (MSD) platform. A Streptavidin coated MSD plate was blocked with 5% Blocker-A in PBS. The plate was washed and different dilutions of standard and PK samples were distributed in the plate. A mixture of a biotinylated anti-sulfamidase mouse monoclonal antibody and Sulfo-Ru-tagged rabbit anti-sulfamidase antibodies was added and the plate was incubated at RT. Complexes of sulfamidase and labelled antibodies will bind to the Streptavidin coated plate via the biotinylated mAb. After washing, the amount of bound complexes was determined by adding a read buffer to the wells and the plate was read in a MSD S12400 instrument. The recorded ECL counts were proportional to the amount of sulfamidase in the sample and evaluated against a relevant sulfamidase standard.

Quantification of Alpha-L-Iduronidase and Modified Alpha-L-Iduronidase by ECL:

Alpha-L-iduronidase and modified alpha-L-iduronidase in plasma PK samples were determined by ECL immunoassay using the Meso Scale Discovery (MSD) platform. The wells of a 96 well streptavidin gold plate (#L155A-1, MesoScaleDiscovery (MSD)) were blocked with 1% Fish Gelatin in Phosphate buffer saline (PBS), washed with wash buffer (PBS+0.05% Tween-20) and incubated with a biotinylated, affinity purified goat-a-human alpha-L-iduronidase polyclonal antibody (BAF2449, R&D) after washing different dilutions of standard and PK samples in sample diluent (1% Fish Gelatin in PBS+0.05% Tween 20+1% C57BL6 serum pool) were incubated in the plate at 700 rpm shake and RT for 2 h. The plate was washed and a alpha-L-iduronidase specific Rutenium (SULFO-TAG, MSD) tagged goat polyclonal antibody (AF2449, R&D) was added and allowed to bind to the captured alpha-L-iduronidase or chemically modified alpha-L-iduronidase. The plate was washed and 2× Read Buffer (MSD) was added. The plate content was analyzed using a MSD Sector 2400 Imager Instrument. The instrument applies a voltage to the plate electrodes, and the SULFO-TAG label, bound to the electrode surface via the formed immune complex, will emit light. The instrument measures the intensity of the emitted light which is proportional to the amount of alpha-L-iduronidase or chemically modified alpha-L-iduronidase in the sample. The amount of alpha-L-iduronidase or chemically modified alpha-L-iduronidase was determined against a relevant alpha-L-iduronidase or chemically modified alpha-L-iduronidase standard.

Quantification of Iduronate 2-Sulfatase and Modified Iduronate 2-Sulfatase by ECL:

Iduronate 2-sulfatase and modified iduronate 2-sulfatase in plasma PK samples were determined by ECL immunoassay using the Meso Scale Discovery (MSD) platform. The wells of a 96 well streptavidin gold plate (#L155A-1, MesoScaleDiscovery (MSD)) were blocked with 1 Fish Gelatin in Phosphate buffer saline (PBS), washed with wash buffer (PBS+0.05% Tween-20) and incubated with a biotinylated, affinity purified goat-a-human iduronate 2-sulfatase polyclonal antibody (BAF2449, R&D) after washing different dilutions of standard and PK samples in sample diluent (1% Fish Gelatin in PBS+0.05% Tween 20+1% C57BL6 serum pool) were incubated in the plate at 700 rpm shake and RT for 2 h. The plate was washed and a iduronate 2-sulfatase specific Rutenium (SULFO-TAG, MSD) tagged goat polyclonal antibody (AF2449, R&D) was added and allowed to bind to the captured iduronate 2-sulfatase or chemically modified iduronate 2-sulfatase. The plate was washed and 2× Read Buffer (MSD) was added. The plate content was analyzed using a MSD Sector 2400 Imager Instrument. The instrument applies a voltage to the plate electrodes, and the SULFO-TAG label, bound to the electrode surface via the formed immune complex, will emit light. The instrument measures the intensity of the emitted light which is proportional to the amount of iduronate 2-sulfatase or chemically modified iduronate 2-sulfatase in the sample. The amount of iduronate 2-sulfatase or chemically modified iduronate 2-sulfatase was determined against a relevant iduronate 2-sulfatase or chemically modified iduronate-2-sulfatase standard.

Results

The plasma/serum clearance in mice of modified sulfamidase, iduronate 2-sulfatase and alpha-L-iduronidase as compared to unmodified counterparts were reduced significantly, see Table 4 below. This is probably at least partly due to the inhibition of receptor mediated uptake in peripheral tissue following chemical modification.

TABLE 4
Plasma/Serum clearance of lysosomal proteins sulfamidase, alpha-L-
iduronidase and iduronate 2-sulfatase

| | | Plasma/Serum |
|---|---|---|
| | Dose | CL |
| Test article | (mg/kg) | (mL/(h · kg)) |
| sulfamidase (SEQ ID NO: 44) | 10 | 170 |
| modified sulfamidase (New method 1) | 10 | 14 |
| iduronate 2-sulfatase (SEQ ID NO: 35) | 1 | 60 |
| modified iduronate-2-sulfatase (New method 2) | 1 | 14 |
| alpha-L-iduronidase (SEQ ID NO: 38) | 3 | 130 |
| modified alpha-L-iduronidase (new method 3) | 3 | 45 |

### Example 7

### In Vivo Effect of Modified Sulfamidase on Brain Heparan Sulfate Storage

Materials and Methods

The effect of intravenously (i.v.) administrated modified sulfamidase produced as described in the general material and methods section, in Quattromed Cell Factory (QMCF) episomal expression system (Icosagen AS) and modified according to new method 1 of Example 3 on brain heparan sulfate storage in vivo was investigated.

Test Article Preparation:

Modified sulfamidase was formulated at 6 mg/mL, sterile filtrated and frozen at −70° C. until used. Frozen modified sulfamidase and corresponding vehicle solution were thawed on the day of injection at RT for minimum one hour up to two hours before use. Chlorpheniramine was dissolved in isotonic saline to a concentration of 0.5 mg/mL, and stored at −20° C.

Animals:

Male mice having a spontaneous homozygous mutation at the mps3a gene, B6.Cg-Sgsh^{mps3a}/PstJ (MPS IIIA)(Jackson Laboratories, ME, USA), were used. The animals were housed singly in cages at 23±1° C. and 40-60% humidity, and had free access to water and standard laboratory chow. The 12/12 h light/dark cycle was set to lights on at 7 pm. The animals were conditioned for at least two weeks before initiating the study. Wild-type siblings from the same breeding unit were also included as controls. In study A, mice were 23-24 weeks old whereas mice were 9-10 weeks old in study B.

Experimental Procedure Study A:

Modified sulfamidase at 30 mg/kg (n=8) and vehicle (n=7) were administered intravenously to MPS IIIA mice every other day for twenty-five days (13 injections). Chlorpheniramine was dosed (2.5 mg/kg) subcutaneously 30-45 min before administration of modified sulfamidase or vehicle. Dosing started approximately at 07.00 in the morning. The test article and vehicle were administered at 5 mL/kg. The final administration volume was corrected for the actual body weight at each dosing occasion. This scheme was repeated for vehicle. The study was finished 2 h after the last injection. Untreated age-matched wild-type mice (n=5) were included in conjunction with the test article-treated groups. The mice were anaesthetized by isoflurane. Blood was withdrawn from retro-orbital plexus bleeding. Perfusion followed by flushing 20 mL saline through the left ventricle of the heart. Tissues were dissected (brain, liver, spleen, lung, and heart), weighed and frozen rapidly in liquid nitrogen. The tissues and blood were prepared to measure hexosamine N-sulfate [α-1,4] uronic acid (HNS-UA) levels using LC-MS/MS. HNS-UA is a disaccharide marker of heparan sulfate storage, and thus a decrease in HNS-UA levels reflects degradation of heparan sulfate. The HNS-UA data were calculated in relative units vs. internal standard, expressed per mg tissue and normalized to the average of the control group. The data were analyzed by one-way ANOVA test and if overall significance was demonstrated also by Bonferroni's multiple comparison post-hoc test for test of significance between groups (*P<0.05, **P<0.01, ***P<0.001).

Experimental Procedure Study B:

Modified sulfamidase at 30 mg/kg (n=6), 10 mg/kg (n=6) and vehicle (n=6) were administered intravenously to MPS IIIA mice once weekly for 10 weeks (10 injections). Chlorpheniramine was dosed (2.5 mg/kg) subcutaneously 30-45 min before administration of modified sulfamidase or vehicle. The final administration volume was corrected for the actual body weight at each dosing occasion. This scheme was repeated for vehicle. The study was finished 24 h after the last injection. Untreated age-matched wild-type mice (n=6) were included in conjunction with the test article-treated groups. The mice were anaesthetized by isoflurane. Blood was withdrawn from retro-orbital plexus bleeding. Perfusion followed by flushing 20 mL saline through the left ventricle of the heart. Tissues were dissected (brain, liver, spleen), weighed and frozen rapidly in liquid nitrogen. The tissues and blood were prepared to measure HNS-UA levels using LC-MS/MS. The HNS-UA data were calculated in relative units vs. internal standard, expressed per mg tissue and normalized to the average of the control group. The data were analyzed by one-way ANOVA test and if overall significance was demonstrated also by Bonferroni's multiple comparison post-hoc test for test of significance between groups (*P<0.05, **P<0.01, ***P<0.001).

LC-MS/MS Analysis of HNS-UA in Tissue Samples:

Liquid chromatography tandem mass spectrometry (LC-MS/MS) analysis of hexosamine N-sulfate [α-1,4] uronic acid (HNS-UA) in tissue samples was conducted partly according to methods described by Fuller et al (Pediatr Res 56: 733-738 (2004)) and Ramsay et al (Mol Genet Metab 78:193-204 (2003)). The tissues (90-180 mg) were homogenized in substrate buffer (29 mM diethylbarbituric acid, 29 mM sodium acetate, 0.68% (w/v) NaCl, 100 mL water, pH 6.5) using a Lysing Matrix D device (MP Biomedicals, LLC, Ohio, US). Homogenization was performed for 25 s in a Savant FastPrep FP120/Bio101 homogenizer (LabWrench, ON, Canada) and the homogenate was subsequently centrifuged in an Eppendorf centrifuge 5417R at 10000 rcf. The supernatant was evaporated to near dryness. 150 μL derivatizing solution (250 mM 3-methyl-1-phenyl-2-pyrazolin-5-one (PMP), 400 mM NH₃, pH 9.1) and 5 μL of the internal standard Chondroitin disaccharide Δdi-4S sodium (ΔUA-GalNAc4S, 0.1 mg/mL) stock solution was added. The derivatization was performed at 70° C. for 90 min under agitation and then the solutions were acidified with 200 μL of 800 mM formic acid. Deionized water was added to the samples to a final volume of 500 μL, and extraction was performed with chloroform (3×500 μL) to remove excess PMP. Centrifugation was performed at 13000×g for 5 min and the upper phase was transferred to a new vial. To remove any excess of formic acid and NH₄COOH, the aqueous phase was evaporated to dryness in a speed vac (Savant Instruments Inc., Farmingdale, N.Y.). The samples were reconstituted to a total of 100 μL of 5% acetonitrile/0.1% acetic acid/0.02% TFA.

LC-MS/MS analysis was performed on Waters Ultra Performance Liquid Chromatography (UPLC), coupled to Sciex API 4000 triple quadrupole mass spectrometer. Instrument control, data acquisition and evaluation were done with Analyst software.

LC separation was performed by the use of an Acquity C18 CSH column (50×2.1 mm, 1.7 μm). Mobile phase A consisted of 5% acetonitrile/0.5% formic acid, and mobile phase B consisted of 95% acetonitrile/0.5% formic acid. A gradient from 1% to 99% B in 7 min was used at a flow rate of 0.35 mL/min. The injection volume was 10 μL. The API 4000 was operated in electrospray negative ion multiple reaction monitoring (MRM) mode. The ion spray voltage was operated at 4.5 kV, and the source temperature was 450° C. Argon was used as collision gas. Collision energy was 34 V. The MRM transitions were 764.4/331.2 (PMP-HNS-UA) and 788.3/534.3 (PMP-internal standard). The relative amount of the HNS-UA was calculated with respect to the level of the internal standard.

Results

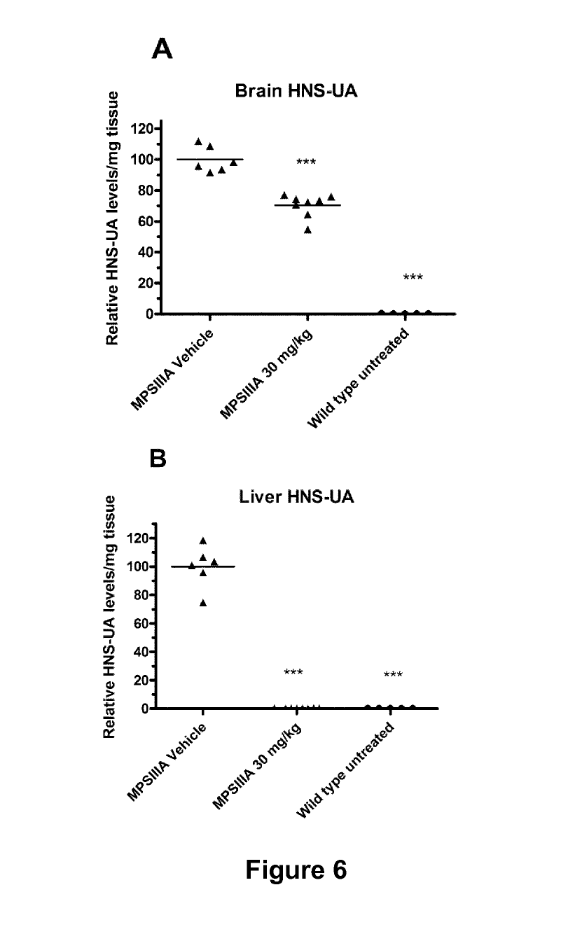

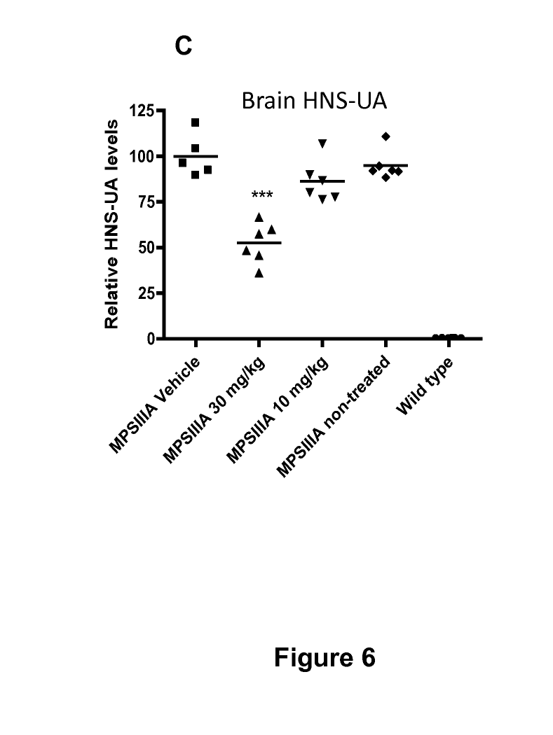

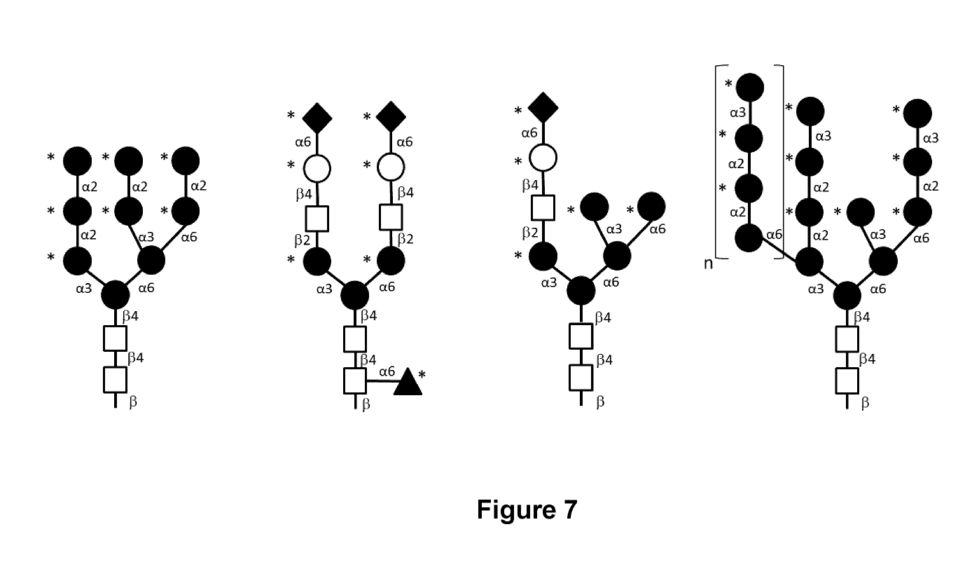

The results from study A shown in FIG. 6A illustrates that sulfamidase modified according to the new method 1 decreased the levels of HNS-UA in the brain by 30% following repeated intravenous administration every other day for 25 days (13 doses) at 30 mg/kg.

In addition, treatment with the modified sulfamidase totally abolished HNS-UA levels in liver (FIG. 6B) and lung (not shown).

The results from study B are shown in FIG. 6C and illustrates that modified sulfamidase according to the new method 1 decreased the levels of HNS-UA in the brain by 48% and 14% following repeated intravenous administration once weekly for 10 weeks at 30 mg/kg and 10 mg/kg, respectively.

These results thus demonstrate that a sulfamidase protein modified according to the new method 1 described herein causes, after long-term treatment, a robust reduction of HNS-UA levels in brain as well as an essentially complete reduction of HNS-UA levels in peripheral organs.

### Example 8

### Optimization of Sulfamidase Modification

The chemical modification process can generally be divided into two parts where the oxidation step is the first step, denoted R1 hereinafter, and the reduction is the second step, denoted R2. To optimize the two steps a full factor design of experiment (DoE) investigating the effect of temperature, concentration and time for the two steps was set up.

Materials and Methods

Sulfamidase produced as described in Example 1 in Quattromed Cell Factory (QMCF) episomal expression system (Icosagen AS) were modified essentially as described in Example 3 for new method 1, however parameters subjected to investigation were varied in accordance with Table 5 (below). The investigation of R1 was carried out with the same reduction and parameters work-up as described in Example 3 (new method 1). The end-points for the analysis are degree of oxidation of glycans described in Example 2, and the level of cell uptake of the modified protein, described in Example 5.

TABLE 5
Parameters varied in R1 and R2

| Variable | R1 | R2 |
|---|---|---|
| T (° C.) | 0, 8, 22 | 0, 8, 22 |
| t (min) | 30, 60, 120 | 30, 60, 120 |
| c (mol/L) | 10, 20, 40 | 1.2x (c in R1), 2.5x (c in R1), 5x (c in R1) |

The number of parameters and the type of design selected yields ten experiments for each step, the results of which were evaluated using the MODDE 10 software (Umetrics AB).

In addition the influence of the second quenching step was tested on sulfamidase produced with the R1 parameters 8° C., 60 min and 20 mM sodium meta-periodate. Two additional reactions were run in parallel to the DoE experiment and quenched using 0.1 M acetone or by addition of acetic acid until a pH of 6.0 or lower was obtained. The final work-up followed the scheme for the other reactions. The sulfamidase thus produced was evaluated using the SDS-PAGE method described in Example 2.

The R2 experiments were conducted with sulfamidase modified according to the parameters found to be optimal after the analysis of the DoE of R1.

Results

The R1 results are summarized in table 6 below:

TABLE 6
R1 experiments and results

| | | | | | | | Cell uptake |
|---|---|---|---|---|---|---|---|
| | | | Remaining original | | | | % of |
| | | | (natural) N-Glycan | | | | unmodified |
| Varied parameters | | | (%) | | | | sulfamidase |
| T (° C.) | t (min) | c (mmol/L) | N (21) | N (131) | N (244) | N (393) | uptake |
| 0 | 30 | 10 | 0 | 0.9 | 0 | 0 | 12 |
| 0 | 120 | 10 | 0 | 0.4 | 0 | 0 | 9.5 |
| 22 | 30 | 10 | 0 | 0.2 | 0 | 0 | 7.1 |
| 22 | 120 | 10 | 0 | 0.1 | 0 | 0 | 8.5 |
| 0 | 30 | 40 | 0 | 0.3 | 0 | 0 | 3.8 |
| 0 | 120 | 40 | 0 | 0.1 | 0 | 0 | 3.5 |
| 22 | 30 | 40 | 0 | 0.1 | 0 | 0 | 2.7 |
| 22 | 120 | 40 | 0 | 0.01 | 0 | 0 | 3.5 |
| 8 | 60 | 20 | 0 | 0.2 | 0 | 0 | 6.1 |
| 8 | 60 | 20 | 0 | 0.2 | 0 | 0 | 6.5 |

In addition, a glycosylation analysis according to Example 2 was conducted for sulfamidase modified according to the known method. No remaining original N-glycans were detected at the N-glycosylation sites N(21), N(131), N(244), and N(393).

The MODDE evaluation of R1 (oxidation) showed that an optimum for R1 at a temperature of around 8° C., a reaction duration of around 1 h and a concentration of around 10 mmol/L of sodium meta-periodate. The overall protein health (e.g. structural integrity) seems to benefit from the lowest oxidant concentration as possible that still limits the cellular uptake via glycan recognition receptors to the level of new method 1 (see Example 5 for details).

Among the various conditions disclosed for R1 reaction time was considered as an important parameter for degree of glycan modification. In addition, periodate concentration may influence degree of glycan modification.

The R2 (reduction) design thus used the above identified preferred parameters for R1, i.e. used for oxidation of sulfamidase. The critical end-point for R2 is FGly content since it was found to influence the activity of the modified sulfamidase (cf Examples 2 and 4). See Table 7 below for results. The relative amount of the peptide fragments containing FGly50 and Ser50 was analyzed with LC-MS by measuring the peak areas from reconstructed ion chromatograms (without correction for ionization efficiency).

TABLE 7
Summary of DoE for R2 and confirmatory experiments

| | | | Active site | |
|---|---|---|---|---|
| Varied parameters | | | Ser formation | FGly/Ser |
| t (min) | T (° C.) | c (mmol/L) | (%) | Ratio |
| 30 | 0 | 12 | 10 | 9.0 |
| 90 | 0 | 12 | 11 | 8.1 |
| 30 | 22 | 12 | 15 | 5.7 |
| 90 | 22 | 12 | 17 | 4.9 |
| 30 | 0 | 50 | 40 | 1.5 |
| 90 | 0 | 50 | 50 | 1.0 |
| 30 | 22 | 50 | 64 | 0.6 |
| 90 | 22 | 50 | 72 | 0.4 |
| 60 | 8 | 25 | 42 | 1.4 |
| 30 | 0 | 20 | 25 | 3.0 |
| 30 | 0 | 50 | 45 | 1.2 |
| 60 | 0 | 15 | 15 | 5.7 |
| 60 | 0 | 25 | 33 | 2.0 |
| 60 | 8 | 12 | 15 | 5.7 |
| 60 | 8 | 50 | 62 | 0.6 |

The DoE for R2 showed that the Ser formation is related to concentration of sodium borohydride and temperature. Taking into account Ser formation and the presence of high molecular weight forms (data not shown, the results are analogous with the ones received for new method 4 in Example 3), the preferred conditions for R2 are a temperature of around 0° C., a reaction duration of around 1 h or less, and a sodium borohydride concentration of more than 12 mmol/L and up to and including 50 mmol/L.

It was confirmed on SDS-PAGE (data not shown) that the sulfamidase produced in a reaction where the reduction step was quenched was comparable with the sulfamidase produced without quenching. This indicates that the introduction of the second quenching step do not negatively affect the quality of the material by either quenching with 0.1 M acetone or by lowering the pH to below 6 by addition of acetic acid.

### Example 9

### Analysis of Glycan Structure after Chemical Modification of Sulfamidase According to Previously Known Method

Material and Methods

Chemical Modification According to the Known Method:

The chemical modification of sulfamidase according to the known method was performed as described in Example 1.

Glycosylation Analysis:

The analysis of glycan structure on sulfamidase after chemical modification was performed according to the LC-MS method described in Example 2.

Resulting modifications on the glycan moieties on the four tryptic peptide fragments containing the N glycosylation sites N(21), N(131), N(244) and N(393) described in Example 2 were investigated by LC-MS analysis.

Results

Glycosylation Analysis:

The type of glycosylation found on the four glycosylation sites prior to the chemical modification was predominantly complex glycans on N(21) and N(393), and oligomannose type of glycans on N(131) and N(244).

After the chemical modification, detailed characterization of the modified glycan structure was performed on the most abundant chemically modified glycopeptides (less abundant glycans were not detectable due to significant decrease in sensitivity as a result of increased heterogeneity of the glycans after chemical modification). In this Example, the modification on Man-6 glycan after chemical modification according to the known method is investigated.

Periodate treatment of glycans cleaves carbon bonds between two adjacent hydroxyl groups of the carbohydrate moieties and alter the molecular mass of the glycan chain. FIG. 8A illustrates an example of predicted bond breaks on mannose after chemical modification. FIG. 8B depicts a model of Man-6 glycan showing the theoretical bond breaks that may take place after oxidation with sodium periodate.

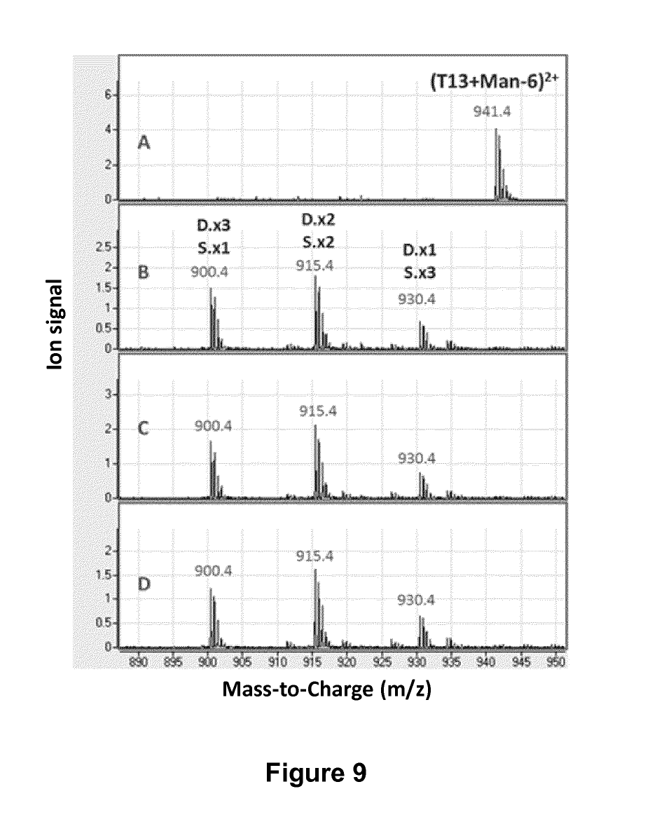

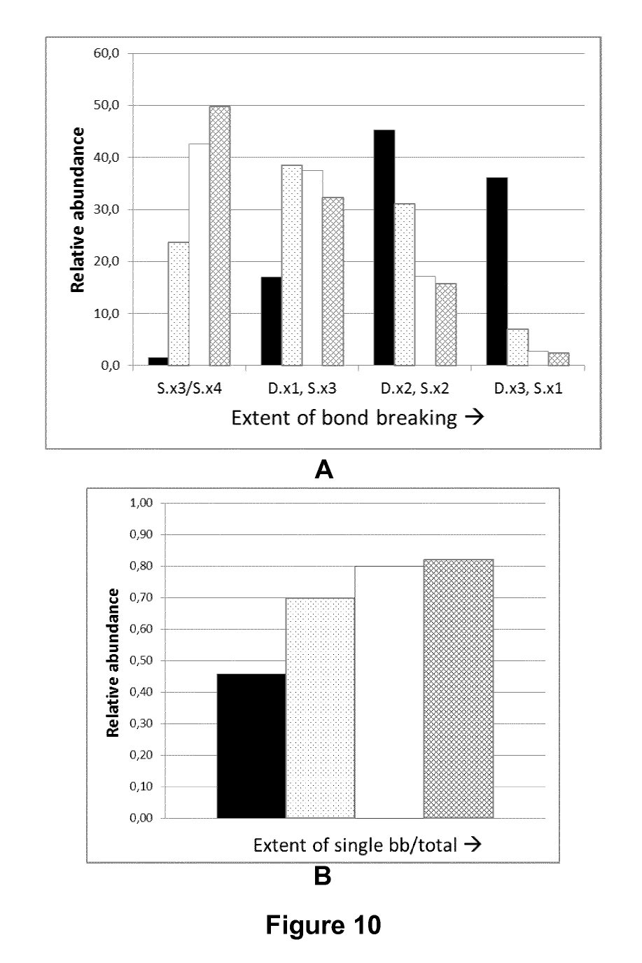

In FIG. 9 are shown mass spectra of the tryptic peptide NITR with Man-6 glycan attached to N(131) (T13+Man-6 glycan), prior to and after chemical modification according to the previously known method. Ions corresponding to the chemically modified glycopeptide with various degree of bond breaking were identified. For Man-6 glycan, there can theoretically be a maximum of 3 double bond breaks and one single bond break. When the modification was performed according to the known method, the most intense ion signal in the mass spectrum was found to be corresponding to 2 double bond breaks and 2 single bond breaks, while the second most intense ion signal corresponded to 3 double bond breaks and one single bond break, which is the most extensive bond breaks possible. A diagram visualizing the extent of bond breaking found on T13+Man-6 glycan after chemical modification according to the known method is shown in FIG. 10A (due to isotopic distribution from the ions observed, the results are approximative but comparable). The reproducibility of the chemical modification was tested by using three different batches of chemically modified sulfamidase produced according to the previously known method. The ions corresponding to different degree of bond breaking showed very similar distribution in the MS spectra from the three different batches.

### Example 10

### Analysis of Glycan Structure after Chemical Modification of Sulfamidase According to New Methods 1, 4, and 5

New Methods 1, 4, and 5:

The chemical modifications of sulfamidase according to the new methods were performed as described in Example 3.

Glycosylation Analysis:

The glycosylation analysis was performed according to the LC-MS method described in Example 2. Resulting modifications on the glycan variants of the four tryptic peptide fragments containing the N glycosylation sites N(21), N(131), N(244) and N(393) were investigated by LC-MS analysis.

Results

Glycosylation Analysis:

Detailed characterization of the modified glycan profile on sulfamidase, chemically modified according to new methods 1, 4, and 5, was performed on the most abundant chemically modified glycopeptides. In this Example 10, the modification on Man-6 glycan after chemical modification according to new methods 1, 4, and 5, was investigated.

Ions corresponding to the chemically modified glycopeptide T13+Man-6 glycan with various degree of bond breaking were identified. Theoretically there can be a maximum of 3 double bond breaks and one single bond break (see FIG. 8B a model of Man-6 glycan showing the bond breaks possible to occur after oxidation with sodium periodate). When the modification was performed according to the new method 1, the most intense ion signal in the mass spectrum was found to be corresponding to one double bond break and 3 single bond breaks, while the second most intense ion signal corresponded to 2 double bond breaks and 2 single bond breaks. When the modification was performed according to new methods 3 and 4, the bond breaks on Man-6 glycan were even further shifted to preferentially single bond breaks. In FIG. 10A is shown a diagram visualizing the extent of bond breaking of the tryptic peptide T13+Man-6 glycan after chemical modification.

The reproducibility of the chemical modification was tested by using triplicates (new method 1) or duplicates (new methods 3) of chemically modified sulfamidase.

When comparing the Man-6 glycan modifications resulting from sulfamidase chemically modified according to the known method with the Man-6 glycan modifications resulting from sulfamidase chemically modified according to the new methods 1, 4, and 5, there was a large difference in degree of bond breaking. This is illustrated in FIG. 10A, where the distribution of the different degrees of bond breaking is plotted for the four methods (due to isotopic distribution from the ions observed, the results are approximative, but comparable).

FIG. 10B shows the relative abundance of single bond breaks for the methods used. The previously known method provides a modified sulfamidase having 45% single bond breaks in the investigated Man-6-glycan, while the new methods 1, 3, and 4 have 70, 80, and 82% single bond breaks, respectively, after chemical modification.

### Example 11

### Analyses of Enzymatic Activity of Iduronate 2-Sulfatase Modified According to Known Method

Material and Methods

Catalytic activity of iduronate 2-sulfatase modified according to known method as described in Example 1 was assessed by incubating preparations of iduronate 2-sulfatase with the substrate 4-Methylumbeliferone iduronide-sulphate. The concentration of substrate in the reaction mixture was 50 μM and the assay buffer was 50 mM sodium acetate, 0.005% Tween 20, 0.1% BSA, 0.025% Anapoe X-100, 1.5 mM sodium azide, pH 5. After the incubation, further desulphation was inhibited by addition of a stop buffer containing 0.4 M sodium phosphate, 0.2 M citrate pH 4.5. A second 24 hour incubation with iduronate 2-sulfatase (assay concentration 0.83 μg/mL) was performed to hydrolyze the product (4-methylumbeliferone iduronide) and release 4-Methylumbeliferone, which was monitored by fluorescence at 460 nm after quenching the reaction with 0.5 M sodium carbonate, 0.025% Triton X-100, pH 10.7.

Results

The activity of iduronate 2-sulfatase modified according to the known method was below 50% of that of unmodified iduronate 2-sulfatase (results not shown).

### Example 12

### Analyses of Enzymatic Activity of Iduronate 2-Sulfatase Modified According to New Methods

Material and Methods

Iduronate 2-sulfatase was modified according to new methods 10 and 11, which are as Example 3 but with the difference that the sodium borohydride reaction mixtures were held at 0° C. for 0.5 h. In new method 11, further the periodate oxidation was performed in the presence of 0.5 mg/mL heparin. Catalytic activity of iduronate 2-sulfatase modified according to new methods 10 and 11 was determined according to the procedure described in Example 11.

Results

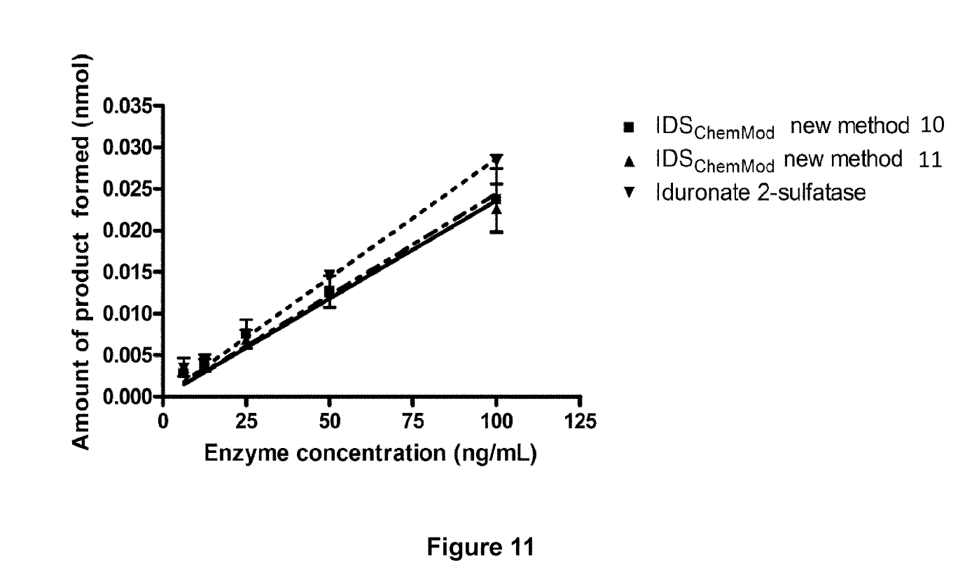

Iduronate 2-sulfatase prepared according to new method 10 and 11 showed an activity that was comparable to that of unmodified iduronate 2-sulfatase (FIG. 11).

### Example 13

### Chemical Modification of Alpha-L-Iduronidase in the Presence of an Active Site Protecting Ligand

As described in Example 3 new method 9, the oxidation (step a)) was performed in the presence of different ligands. The ligands used were 4-methylumbeliferone iduronide, 5-fluoro-α-l-idopyranosyluronic acid fluoride, heparin, heparin sulphate and D-Saccaric acid 1.4-lactone, respectively.

Enzymatic activity was measured as described in “Standardization of α-L-iduronidase Enzyme Assay with Michaelis-Menten Kinetics. Ou L, Herzog T L, Carrie M. Wilmot CM3, and Chester B. Whitley C B. Mol Genet Metab. 2014 111: 113-115”.

Results

When 5-fluoro-α-l-idopyranosyluronic acid fluoride was used as a protecting ligand during step a) a 52% lower catalytic activity was obtained for the modified alpha-L-iduronidase compared to when step a) was performed without a protecting ligand i.e. according to new method 8. When other inhibitors known in the literature such as D-Saccaric acid 1.4-lactone was used a 25% decrease in catalytic activity was obtained for the modified alpha-L-iduronidase. A similar trend of decrease in catalytic activity was noted for substrates such as 4-MU-iduronide, heparin or heparin sulphate (data not shown).

### Example 14

### Chemical Modification of Alpha-L-Iduronidase Immobilized on a Gel Matrix

The modification method as described herein, and in particular, new method 3 of Example 3, was performed while alpha-L-iduronidase was immobilized on a gel matrix. Alpha-L-iduronidase was immobilized by loading the SOURCE™ 15S Strong Cation Exchange column with a 20 mM sodium phosphate buffer with 20 mM NaCl and a pH of 6.7.

Aldurazyme was incubated with 250 μL Source 15S gel matrix for 1 hour. After that the gel matrix was gently pelleted and concentration of protein in supernatant was determined to be below 10% of that before incubation with gel. One sample was stored stored in a refrigerator one day before proceeding with chemical modification. A second incubation was made just prior to chemical modification.

Following loading of alpha-L-iduronidase, the column was equilibrated with solutions for step a), quenching of step a), step b), and quenching of step b) in a consecutive fashion. Elution of chemically modified alpha-L-iduronidase is performed by washing the column with a buffer containing 100 mM sodium phosphate and 700 mM sodium chloride with a pH of 5.6.

Enzymatic activity was measured as described in “Standardization of α-L-iduronidase Enzyme Assay with Michaelis-Menten Kinetics. Ou L, Herzog T L, Carrie M. Wilmot CM3, and Chester B. Whitley C B. Mol Genet Metab. 2014 111: 113-115”.

Results Binding in Batch Mode to Source 15S

Performing the chemical modification while Aldurazyme was immobilized on a gel matrix gave an 8% increased catalytic activity of the resulting modified alpha-L-iduronidase compared to when the modification was performed in solution.

### Example 15

### Chemical Modification of Alpha-L-Iduronidase Immobilized on a Gel Matrix and in the Presence of a Protecting Ligand

The modification method as described herein, and in particular, new method 3 of Example 3, was performed while alpha-L-iduronidase was immobilized on a gel matrix and in the presence of a ligand. The ligands used were 5-fluoro-α-l-idopyranosyluronic acid fluoride and D-Saccaric acid 1.4-lactone, respectively.

Alpha-L-iduronidase was immobilized by loading the Source 15S Strong Cation Exchange column with a 20 mM sodium phosphate buffer with 20 mM NaCl and a pH of 6.7. Aldurazyme was incubated with 250 μL Source 15S gel matrix for 1 hour. After that the gel matrix was gently pelleted and concentration of protein in supernatant was determined to be below 10% of that before incubation with gel. One sample was stored stored in a refrigerator one day before proceeding with chemical modification. A second incubation was made just prior to chemical modification. Following loading of alpha-L-iduronidase, the column was equilibrated with solutions for step a), quenching of step a), step b), and quenching of step b) in a consecutive fashion. Elution of chemically modified alpha-L-iduronidase was performed by washing the column with a buffer containing 100 mM sodium phosphate and 700 mM sodium chloride with a pH of 5.6.

Enzymatic activity was measured as described in “Standardization of α-L-iduronidase Enzyme Assay with Michaelis-Menten Kinetics. Ou L, Herzog T L, Carrie M. Wilmot CM3, and Chester B. Whitley C B. Mol Genet Metab. 2014 111: 113-115”.

Results

The combined approach of using a inhibitor to protect the active site in combination with immobilization of aldurazyme on a gel matrix gave the surprising finding that 5-fluoro-α-l-idopyranosyluronic acid fluoride in combination of immobilization on a Source 15S Strong Cation Exchange column yielded an increase of 37% of catalytic activity of the resulting modified alpha-L-iduronidase compared to when the modification was performed in solution without a protective ligand. The corresponding result when using the inhibitor D-Saccaric acid 1.4-lactone was a 25% decrease in catalytic activity compared to when the modification was performed in solution without a protective ligand.

### Example 16

### Distribution of Modified Iduronate 2-Sulfatase to Brain of Iduronate 2-Sulfatase Deficient Mice

Materials and Methods

The distribution of intravenously (iv) administrated modified iduronate 2-sulfatase produced according to new method 2 of Example 4 to brain in vivo was investigated.

Test Article Preparation:

Modified iduronate 2-sulfatase was formulated at 2 mg/mL, sterile filtrated and frozen at −70° C. until used.

Animals:

Male mice, IDS-KO (B6N.Cg-Idstm1Muen/J)(Jackson Laboratories, ME, USA), were used. The animals were housed singly in cages at 23±1° C. and 40-60% humidity, and had free access to water and standard laboratory chow. The 12/12 h light/dark cycle was set to lights on at 7 pm. The animals were conditioned for at least two weeks before initiating the study. The mice were given an intravenous administration in the tail vein of 10 mg/kg modified iduronate 2-sulfatase. The study was finished 24 h after the last injection. The mice were anaesthetized by isoflurane. Blood was withdrawn from retro-orbital plexus bleeding. Perfusion followed by flushing 20 mL saline through the left ventricle of the heart. Brain was dissected weighed and frozen rapidly in liquid nitrogen. Brain homogenates where prepared and activity was assessed using the method described in example 2 with addition of 10 mM lead acetate in the assay buffer as adjustment to the protocoll.

Results: Activity of modified iduronate 2-sulfatase in perfused brain homogenates of IDS-KO mice could be confirmed. An average activity of 1.8±0.4 μM/min (n=4) was determined under the assay conditions used.

1. A method of preparing a modified lysosomal protein, said method comprising:
a) reacting a glycosylated lysosomal protein with an alkali metal periodate for a time of no more than 4 h; and
b) reacting said lysosomal protein with an alkali metal borohydride for a time period of no more than 2 h;
thereby modifying glycan moieties of the lysosomal protein and reducing the activity of the lysosomal protein with respect to glycan recognition receptors, provided that said protein is not sulfamidase.

2. The method of claim 1, wherein step b) is further characterized by at least one of:
i) said alkali metal borohydride is sodium borohydride;
ii) said borohydride is used at a concentration of no more than 4 times the concentration of said periodate;
iii) said reaction is performed for a time period of no more than 2 h; and
iv) said reaction is performed at a temperature of between 0 and 8° C.

3. The method according to claim 1, wherein step a) is further characterized by at least one of:
i) said alkali metal periodate is sodium meta-periodate;
ii) said periodate is used at a concentration of no more than 20 mM;
iii) said reaction is performed at a temperature of between 0 and 22° C.;
iv) said reaction is performed for a time period of no more than 3 h; and
v) said reaction of step a) is performed at a pH of 3-7.

4. The method according to claim 1, wherein step a) is performed for a time period of no more than 3 h and step b) is performed for no more than 1 h, and said borohydride optionally is used at a concentration of no more than 4 times the concentration of said periodate.

5. The method according to claim 1, wherein step a) and step b) are performed in sequence without performing any dialysis, ultrafiltration, precipitation or buffer exchange.

6. A method of preparing a modified lysosomal protein, said method comprising:
a) reacting a glycosylated lysosomal protein with an alkali metal periodate, and
b) reacting said lysosomal protein with an alkali metal borohydride;
thereby modifying glycan moieties of the lysosomal protein and reducing the activity of the lysosomal protein with respect to glycan recognition receptors, wherein the active site or functional epitope of said lysosomal protein is made inaccessible to oxidative and/or reductive reactions during at least one of steps a) and b).

7. The method according to claim 6, wherein step b) is further characterized by at least one of:
i) said alkali metal borohydride is sodium borohydride;
ii) said borohydride is used at a concentration of no more than 4 times the concentration of said periodate;
iii) said reaction is performed for a time period of no more than 2 h; and
iv) said reaction is performed at a temperature of between 0 and 8° C.

8. The method according to claim 6, wherein step a) is further characterized by at least one of:
i) said alkali metal periodate is sodium meta-periodate;
ii) said periodate is used at a concentration of no more than 20 mM;
iii) said reaction is performed at a temperature of between 0 and 22° C.;
iv) said reaction is performed for a time period of no more than 3 h; and
v) said reaction of step a) is performed at a pH of 3-7.

9. The method according to claim 6, wherein step a) is performed for a time period of no more than 3 h and step b) is performed for no more than 1 h, and said borohydride optionally is used at a concentration of no more than 4 times the concentration of said periodate.

10. The method according to claim 6, wherein step a) and step b) are performed in sequence without performing any dialysis, ultrafiltration, precipitation or buffer exchange.

11. The method according to claim 1, wherein said modified lysosomal protein is a sulfatase; a glycoside hydrolase, or a protease.

12. The method according to claim 1, wherein the lysosomal protein is selected from deoxyribonuclease-2-alpha; beta-mannosidase; ribonuclease T2; lysosomal alpha-mannosidase; alpha L-iduronidase; tripeptidyl-peptidase 1; hyaluronidase-3; cathepsin L2; ceroid-lipofuscinosis neuronal protein 5; glucosylceramidase; tissue alpha-L-fucosidase; myeloperoxidase; alpha-galactosidase A; beta-hexosaminidase subunit alpha; cathepsin D; prosaposin; beta-hexosaminidase subunit beta; cathepsin L1; cathepsin B; beta-glucuronidase; pro-cathepsin H; non-secretory ribonuclease; lysosomal alpha-glucosidase; lysosomal protective protein; gamma-interferon-inducible lysosomal thiol reductase; tartrate-resistant acid phosphatase type 5; arylsulfatase A; prostatic acid phosphatase; N-acetylglucosamine-6-sulfatase; arylsulfatase B; beta-galactosidase; alpha-N-acetylgalactosaminidase; sphingomyelin phosphodiesterase; ganglioside GM2 activator; N(4)-(beta-N-acetylglucosaminyl)-L-asparaginase; iduronate 2-sulfatase; cathepsin S; N-acetylgalactosamine-6-sulfatase; lysosomal acid lipase/cholesteryl ester hydrolase; lysosomal Pro-X carboxypeptidase; cathepsin O; cathepsin K; palmitoyl-protein thioesterase 1; arylsulfatase D; dipeptidyl peptidase 1; alpha-N-acetylglucosaminidase; galactocerebrosidase; epididymal secretory protein E1; di-N-acetylchitobiase; N-acylethanolamine-hydrolyzing acid amidase; hyaluronidase-1; chitotriosidase-1; acid ceramidase; phospholipase B-like 1; proprotein convertase subtilisin/kexin type 9; group XV phospholipase A2; putative phospholipase B-like 2; deoxyribonuclease-2-beta; gamma-glutamyl hydrolase; arylsulfatase G; L-amino-acid oxidase; sialidase-1; legumain; sialate O-acetylesterase; thymus-specific serine protease; cathepsin Z; cathepsin F; prenylcysteine oxidase 1; dipeptidyl peptidase 2; lysosomal thioesterase PPT2; heparanase; carboxypeptidase Q; β-glucuronidase, and sulfatase-modifying factor 1.

13. The method according to claim 1, wherein at least one of steps a) and b) of the method is/are performed in the presence of a protective ligand.

14. The method according to claim 1 wherein steps a) and b) of the method are performed while the lysosomal protein is immobilized on a resin.

15. A modified lysosomal protein having a reduced content of unmodified glycan moieties, characterized in that no more than 50% of the glycan moieties remain unmodified as compared to an unmodified form of the lysosomal protein, said protein thereby having a reduced activity for glycan recognition receptors, provided that said protein is not sulfamidase, β-glucuronidase, tripeptidyl peptidase 1 (TPP1) or alpha L-iduronidase.

16. The modified lysosomal protein according to claim 15, said protein being selected from deoxyribonuclease-2-alpha; beta-mannosidase; ribonuclease T2; lysosomal alpha-mannosidase; hyaluronidase-3; cathepsin L2; ceroid-lipofuscinosis neuronal protein 5; glucosylceramidase; tissue alpha-L-fucosidase; myeloperoxidase; alpha-galactosidase A; beta-hexosaminidase subunit alpha; cathepsin D; prosaposin; beta-hexosaminidase subunit beta; cathepsin L1; cathepsin B; pro-cathepsin H; non-secretory ribonuclease; lysosomal alpha-glucosidase; lysosomal protective protein; gamma-interferon-inducible lysosomal thiol reductase; tartrate-resistant acid phosphatase type 5; arylsulfatase A; prostatic acid phosphatase; N-acetylglucosamine-6-sulfatase; arylsulfatase B; beta-galactosidase; alpha-N-acetylgalactosaminidase; sphingomyelin phosphodiesterase; ganglioside GM2 activator; N(4)-(beta-N-acetylglucosaminyl)-L-asparaginase; iduronate 2-sulfatase; cathepsin S; N-acetylgalactosamine-6-sulfatase; lysosomal acid lipase/cholesteryl ester hydrolase; lysosomal Pro-X carboxypeptidase; cathepsin O; cathepsin K; palmitoyl-protein thioesterase 1; arylsulfatase D; dipeptidyl peptidase 1; alpha-N-acetylglucosaminidase; galactocerebrosidase; epididymal secretory protein E1; di-N-acetylchitobiase; N-acylethanolamine-hydrolyzing acid amidase; hyaluronidase-1; chitotriosidase-1; acid ceramidase; phospholipase B-like 1; proprotein convertase subtilisin/kexin type 9; group XV phospholipase A2; putative phospholipase B-like 2; deoxyribonuclease-2-beta; gamma-glutamyl hydrolase; arylsulfatase G; L-amino-acid oxidase; sialidase-1; legumain; sialate O-acetylesterase; thymus-specific serine protease; cathepsin Z; cathepsin F; prenylcysteine oxidase 1; dipeptidyl peptidase 2; lysosomal thioesterase PPT2; heparanase; carboxypeptidase Q, and sulfatase-modifying factor 1.

17. The modified lysosomal protein according to claim 15, wherein no more than 45% of the glycan moieties remain unmodified compared to an unmodified form of the lysosomal protein.

18. The modified lysosomal protein according to claim 15, wherein unmodified glycan moieties of said lysosomal protein are disrupted by single bond breaks and double bond breaks, the extent of single bond breaks being at least 60% in oligomannose glycans.

19. The modified lysosomal protein according to claim 15, wherein said unmodified glycan moieties are absent from at least one N-glycosylation site of said lysosomal protein.

20. The modified lysosomal protein according to claim 15, wherein said lysosomal protein has retained catalytic activity of that of the corresponding unmodified lysosomal protein.

21. A modified lysosomal protein obtainable by the method according to claim 1, provided that said protein is not sulfamidase.

22. (canceled)

23. (canceled)

24. A method of treating a mammal afflicted with a lysosomal storage disease comprising administering to the mammal a therapeutically effective amount of a modified lysosomal protein according to claim 15.


SEQUENCE LISTING (74 sequences)

SEQ ID NO: 1 - protein, 342 aa, HOMO SAPIENS
CYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 2 - protein, 862 aa, HOMO SAPIENS
AAELSYSLRGNWSICNGNGSLELPGAVPGCVHSALFQQGLIQDSYYRFNDLNYRWVSLDNWTYSKEFKIPFEISKWQKVNLILEGVDTVSKILFNEVTIGETDNMFNRYSFDITNVVRDVNSIELRFQSAVLYAAQQSKAHTRYQVPPDCPPLVQKGECHVNFVRKEQCSFSWDWGPSFPTQGIWKDVRIEAYNICHLNYFTFSPIYDKSAQEWNLEIESTFDVVSSKPVGGQVIVAIPKLQTQQTYSIELQPGKRIVELFVNISKNITVETWWPHGHGNQTGYNMTVLFELDGGLNIEKSAKVYFRTVELIEEPIKGSPGLSFYFKINGFPIFLKGSNWIPADSFQDRVTSELLRLLLQSVVDANMNTLRVWGGGIYEQDEFYELCDELGIMVWQDFMFACALYPTDQGFLDSVTAEVAYQIKRLKSHPSIIIWSGNNENEEALMMNWYHISFTDRPIYIKDYVTLYVKNIRELVLAGDKSRPFITSSPTNGAETVAEAWVSQNPNSNYFGDVHFYDYISDCWNWKVFPKARFASEYGYQSWPSFSTLEKVSSTEDWSFNSKFSLHRQHHEGGNKQMLYQAGLHFKLPQSTDPLRTFKDTIYLTQVMQAQCVKTETEFYRRSRSEIVDQQGHTMGALYWQLNDIWQAPSWASLEYGGKWKMLHYFAQNFFAPLLPVGFENENTFYIYGVSDLHSDYSMTLSVRVHTWSSLEPVCSRVTERFVMKGGEAVCLYEEPVSELLRRCGNCTRESCVVSFYLSADHELLSPTNYHFLSSPKEAVGLCKAQITAIISQQGDIFVFDLETSAVAPFVWLDVGSIPGRFSDNGFLMTEKTRTILFYPWEPTSKNELEQSFHVTSLTDIY

SEQ ID NO: 3 - protein, 232 aa, HOMO SAPIENS
DKRLRDNHEWKKLIMVQHWPETVCEKIQNDCRDPPDYWTIHGLWPDKSEGCNRSWPFNLEEIKDLLPEMRAYWPDVIHSFPNRSRFWKHEWEKHGTCAAQVDALNSQKKYFGRSLELYRELDLNSVLLKLGIKPSINYYQVADFKDALARVYGVIPKIQCLPPSQDEEVQTIGQIELCLTKQDQQLQNCTEPGEQPSPKQEVWLANGAAESRGLRVCEDGPVFYPPPKKTKH

SEQ ID NO: 4 - protein, 962 aa, HOMO SAPIENS
GGYETCPTVQPNMLNVHLLPHTHDDVGWLKTVDQYFYGIKNDIQHAGVQYILDSVISALLADPTRRFIYVEIAFFSRWWHQQTNATQEVVRDLVRQGRLEFANGGWVMNDEAATHYGAIVDQMTLGLRFLEDTFGNDGRPRVAWHIDPFGHSREQASLFAQMGFDGFFFGRLDYQDKWVRMQKLEMEQVWRASTSLKPPTADLFTGVLPNGYNPPRNLCWDVLCVDQPLVEDPRSPEYNAKELVDYFLNVATAQGRYYRTNHTVMTMGSDFQYENANMWFKNLDKLIRLVNAQQAKGSSVHVLYSTPACYLWELNKANLTWSVKHDDFFPYADGPHQFWTGYFSSRPALKRYERLSYNFLQVCNQLEALVGLAANVGPYGSGDSAPLNEAMAVLQHHDAVSGTSRQHVANDYARQLAAGWGPCEVLLSNALARLRGFKDHFTFCQQLNISICPLSQTAARFQVIVYNPLGRKVNWMVRLPVSEGVFVVKDPNGRTVPSDVVIFPSSDSQAHPPELLFSASLPALGFSTYSVAQVPRWKPQARAPQPIPRRSWSPALTIENEHIRATFDPDTGLLMEIMNMNQQLLLPVRQTFFWYNASIGDNESDQASGAYIFRPNQQKPLPVSRWAQIHLVKTPLVQEVHQNFSAWCSQVVRLYPGQRHLELEWSVGPIPVGDTWGKEVISRFDTPLETKGRFYTDSNGREILERRRDYRPTWKLNQTEPVAGNYYPVNTRIYITDGNMQLTVLTDRSQGGSSLRDGSLELMVHRRLLKDDGRGVSEPLMENGSGAWVRGRHLVLLDTAQAAAAGHRLLAEQEVLAPQVVLAPGGGAAYNLGAPPRTQFSGLRRDLPPSVHLLTLASWGPEMVLLRLEHQFAVGEDSGRNLSAPVTLNLRDLFSTFTITRLQETTLVANQLREAASRLKWTTNTGPTPHQTPYQLDPANITLEPMEIRTFLASVQWKEVDG

SEQ ID NO: 5 - protein, 544 aa, HOMO SAPIENS
SYSPEPDQRRTLPPGWVSLGRADPEEELSLTFALRQQNVERLSELVQAVSDPSSPQYGKYLTLENVADLVRPSPLTLHTVQKWLLAAGAQKCHSVITQDFLTCWLSIRQAELLLPGAEFHHYVGGPTETHVVRSPHPYQLPQALAPHVDFVGGLHRFPPTSSLRQRPEPQVTGTVGLHLGVTPSVIRKRYNLTSQDVGSGTSNNSQACAQFLEQYFHDSDLAQFMRLFGGNFAHQASVARVVGQQGRGRAGIEASLDVQYLMSAGANISTWVYSSPGRHEGQEPFLQWLMLLSNESALPHVHTVSYGDDEDSLSSAYIQRVNTELMKAAARGLTLLFASGDSGAGCWSVSGRHQFRPTFPASSPYVTTVGGTSFQEPFLITNEIVDYISGGGFSNVFPRPSYQEEAVTKFLSSSPHLPPSSYFNASGRAYPDVAALSDGYWVVSNRVPIPWVSGTSASTPVFGGILSLINEHRILSGRPPLGFLNPRLYQQHGAGLFDVTRGCHESCLDEEVEGQGFCSGPGWDPVTGWGTPNFPALLKTLLNP

SEQ ID NO: 6 - protein, 397 aa, HOMO SAPIENS
QPLPQVPERPFSVLWNVPSAHCEARFGVHLPLNALGIIANRGQHFHGQNMTIFYKNQLGLYPYFGPRGTAHNGGIPQALPLDRHLALAAYQIHHSLRPGFAGPAVLDWEEWCPLWAGNWGRRRAYQAASWAWAQQVFPDLDPQEQLYKAYTGFEQAARALMEDTLRVAQALRPHGLWGFYHYPACGNGWHSMASNYTGRCHAATLARNTQLHWLWAASSALFPSIYLPPRLPPAHHQAFVRHRLEEAFRVALVGHRHPLPVLAYVRLTHRRSGRFLSQDDLVQSIGVSAALGAAGVVLWGDLSLSSSEEECWHLHDYLVDTLGPYVINVTRAAMACSHQRCHGHGRCARRDPGQMEAFLHLWPDGSLGDWKSFSCHCYWGWAGPTCQEPRPGPKEAV

SEQ ID NO: 7 - protein, 317 aa, HOMO SAPIENS
VPKFDQNLDTKWYQWKATHRRLYGANEEGWRRAVWEKNMKMIELHNGEYSQGKHGFTMAMNAFGDMTNEEFRQMMGCFRNQKFRKGKVFREPLFLDLPKSVDWRKKGYVTPVKNQKQCGSCWAFSATGALEGQMFRKTGKLVSLSEQNLVDCSRPQGNQGCNGGFMARAFQYVKENGGLDSEESYPYVAVDEICKYRPENSVANDTGFTVVAPGKEKALMKAVATVGPISVAMDAGHSSFQFYKSGIYFEPDCSSKNLDHGVLVVGYGFEGANSNNSKYWLVKNSWGPEWGSNGYVKIAKDKNNHCGIATAASYPNV

SEQ ID NO: 8 - protein, 312 aa, HOMO SAPIENS
IPSRRHWPVPYKRFDFRPKPDPYCQAKYTFCPTGSPIPVMEGDDDIEVFRLQAPVWEFKYGDLLGHLKIMHDAIGFRSTLTGKNYTMEWYELFQLGNCTFPHLRPEMDAPFWCNQGAACFFEGIDDVHWKENGTLVQVATISGNMFNQMAKWVKQDNETGIYYETWNVKASPEKGAETWFDSYDCSKFVLRTFNKLAEFGAEFKNIETNYTRIFLYSGEPTYLGNETSVFGPTGNKTLGLAIKRFYYPFKPHLPTKEFLLSLLQIFDAVIVHKQFYLFYNFEYWFLPMKFPFIKITYEEIPLPIRNKTLSGL

SEQ ID NO: 9 - protein, 497 aa, HOMO SAPIENS
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 10 - protein, 435 aa, HOMO SAPIENS
QPPRRYTPDWPSLDSRPLPAWFDEAKFGVFIHWGVFSVPAWGSEWFWWHWQGEGRPQYQRFMRDNYPPGFSYADFGPQFTARFFHPEEWADLFQAAGAKYVVLTTKHHEGFTNWPSPVSWNWNSKDVGPHRDLVGELGTALRKRNIRYGLYHSLLEWFHPLYLLDKKNGFKTQHFVSAKTMPELYDLVNSYKPDLIWSDGEWECPDTYWNSTNFLSWLYNDSPVKDEVVVNDRWGQNCSCHHGGYYNCEDKFKPQSLPDHKWEMCTSIDKFSWGYRRDMALSDVTEESEIISELVQTVSLGGNYLLNIGPTKDGLIVPIFQERLLAVGKWLSINGEAIYASKPWRVQWEKNTTSVWYTSKGSAVYAIFLHWPENGVLNLESPITTSTTKITMLGIQGDLKWSTDPDKGLFISLPQLPPSAVPAEFAWTIKLTGVK

SEQ ID NO: 11 - protein, 697 aa, HOMO SAPIENS
AAPAVLGEVDTSLVLSSMEEAKQLVDKAYKERRESIKQRLRSGSASPMELLSYFKQPVAATRTAVRAADYLHVALDLLERKLRSLWRRPFNVTDVLTPAQLNVLSKSSGCAYQDVGVTCPEQDKYRTITGMCNNRRSPTLGASNRAFVRWLPAEYEDGFSLPYGWTPGVKRNGFPVALARAVSNEIVRFPTDQLTPDQERSLMFMQWGQLLDHDLDFTPEPAARASFVTGVNCETSCVQQPPCFPLKIPPNDPRIKNQADCIPFFRSCPACPGSNITIRNQINALTSFVDASMVYGSEEPLARNLRNMSNQLGLLAVNQRFQDNGRALLPFDNLHDDPCLLTNRSARIPCFLAGDTRSSEMPELTSMHTLLLREHNRLATELKSLNPRWDGERLYQEARKIVGAMVQIITYRDYLPLVLGPTAMRKYLPTYRSYNDSVDPRIANVFTNAFRYGHTLIQPFMFRLDNRYQPMEPNPRVPLSRVFFASWRVVLEGGIDPILRGLMATPAKLNRQNQIAVDEIRERLFEQVMRIGLDLPALNMQRSRDHGLPGYNAWRRFCGLPQPETVGQLGTVLRNLKLARKLMEQYGTPNNIDIWMGGVSEPLKRKGRVGPLLACIIGTQFRKLRDGDRFWWENEGVFSMQQRQALAQISLPRIICDNTGITTVSKNNIFMSNSYPRDFVNCSTLPALNLASWREAS

SEQ ID NO: 12 - protein, 398 aa, HOMO SAPIENS
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 13 - protein, 507 aa, HOMO SAPIENS
LWPWPQNFQTSDQRYVLYPNNFQFQYDVSSAAQPGCSVLDEAFQRYRDLLFGSGSWPRPYLTGKRHTLEKNVLVVSVVTPGCNQLPTLESVENYTLTINDDQCLLLSETVWGALRGLETFSQLVWKSAEGTFFINKTEIEDFPRFPHRGLLLDTSRHYLPLSSILDTLDVMAYNKLNVFHWHLVDDPSFPYESFTFPELMRKGSYNPVTHIYTAQDVKEVIEYARLRGIRVLAEFDTPGHTLSWGPGIPGLLTPCYSGSEPSGTFGPVNPSLNNTYEFMSTFFLEVSSVFPDFYLHLGGDEVDFTCWKSNPEIQDFMRKKGFGEDFKQLESFYIQTLLDIVSSYGKGYVVWQEVFDNKVKIQPDTIIQVWREDIPVNYMKELELVTKAGFRALLSAPWYLNRISYGPDWKDFYIVEPLAFEGTPEQKALVIGGEACMWGEYVDNTNLVPRLWPRAGAVAERLWSNKLTSDLTFAYERLSHFRCELLRRGVQAQPLNVGFCEQEFEQT

SEQ ID NO: 14 - protein, 394 aa, HOMO SAPIENS
SALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 15 - protein, 508 aa, HOMO SAPIENS
GPVLGLKECTRGSAVWCQNVKTASDCGAVKHCLQTVWNKPTVKSLPCDICKDVVTAAGDMLKDNATEEEILVYLEKTCDWLPKPNMSASCKEIVDSYLPVILDIIKGEMSRPGEVCSALNLCESLQKHLAELNHQKQLESNKIPELDMTEVVAPFMANIPLLLYPQDGPRSKPQPKDNGDVCQDCIQMVTDIQTAVRTNSTFVQALVEHVKEECDRLGPGMADICKNYISQYSEIAIQMMMHMQPKEICALVGFCDEVKEMPMQTLVPAKVASKNVIPALELVEPIKKHEVPAKSDVYCEVCEFLVKEVTKLIDNNKTEKEILDAFDKMCSKLPKSLSEECQEVVDTYGSSILSILLEEVSPELVCSMLHLCSGTRLPALTVHVTQPKDGGFCEVCKKLVGYLDRNLEKNSTKQEILAALEKGCSFLPDPYQKQCDQFVAEYEPVLIEILVEVMDPSFVCLKIGACPSAHKPLLGTEKCIWGPSYWCQNTETAAQCNAVEHCKRHVWN

SEQ ID NO: 16 - protein, 514 aa, HOMO SAPIENS
ARAPSVSAKPGPALWPLPLSVKMTPNLLHLAPENFYISHSPNSTAGPSCTLLEEAFRRYHGYIFGFYKWHHEPAEFQAKTQVQQLLVSITLQSECDAFPNISSDESYTLLVKEPVAVLKANRVWGALRGLETFSQLVYQDSYGTFTINESTIIDSPRFSHRGILIDTSRHYLPVKIILKTLDAMAFNKFNVLHWHIVDDQSFPYQSITFPELSNKGSYSLSHVYTPNDVRMVIEYARLRGIRVLPEFDTPGHTLSWGKGQKDLLTPCYSRQNKLDSFGPINPTLNTTYSFLTTFFKEISEVFPDQFIHLGGDEVEFKCWESNPKIQDFMRQKGFGTDFKKLESFYIQKVLDIIATINKGSIVWQEVFDDKAKLAPGTIVEVWKDSAYPEELSRVTASGFPVILSAPWYLDLISYGQDWRKYYKVEPLDFGGTQKQKQLFIGGEACLWGEYVDATNLTPRLWPRASAVGERLWSSKDVRDMDDAYDRLTRHRCRMVERGIAAQPLYAGYCNHENM

SEQ ID NO: 17 - protein, 316 aa, HOMO SAPIENS
TLTFDHSLEAQWTKWKAMHNRLYGMNEEGWRRAVWEKNMKMIELHNQEYREGKHSFTMAMNAFGDMTSEEFRQVMNGFQNRKPRKGKVFQEPLFYEAPRSVDWREKGYVTPVKNQGQCGSCWAFSATGALEGQMFRKTGRLISLSEQNLVDCSGPQGNEGCNGGLMDYAFQYVQDNGGLDSEESYPYEATEESCKYNPKYSVANDTGFVDIPKQEKALMKAVATVGPISVAIDAGHESFLFYKEGIYFEPDCSSEDMDHGVLVVGYGFESTESDNNKYWLVKNSWGEEWGMGGYVKMAKDRRNHCGIASAASYPTV

SEQ ID NO: 18 - protein, 322 aa, HOMO SAPIENS
RSRPSFHPLSDELVNYVNKRNTTWQAGHNFYNVDMSYLKRLCGTFLGGPKPPQRVMFTEDLKLPASFDAREQWPQCPTIKEIRDQGSCGSCWAFGAVEAISDRICIHTNAHVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYNSYSVSNSEKDIMAEIYKNGPVEGAFSVYSDFLLYKSGVYQHVTGEMMGGHAIRILGWGVENGTPYWLVANSWNTDWGDNGFFKILRGQDHCGIESEVVAGIPRTDQYWEKI

SEQ ID NO: 19 - protein, 629 aa, HOMO SAPIENS
LQGGMLYPQESPSRECKELDGLWSFRADFSDNRRRGFEEQWYRRPLWESGPTVDMPVPSSFNDISQDWRLRHFVGWVWYEREVILPERWTQDLRTRVVLRIGSAHSYAIVWVNGVDTLEHEGGYLPFEADISNLVQVGPLPSRLRITIAINNTLTPTTLPPGTIQYLTDTSKYPKGYFVQNTYFDFFNYAGLQRSVLLYTTPTTYIDDITVTTSVEQDSGLVNYQISVKGSNLFKLEVRLLDAENKVVANGTGTQGQLKVPGVSLWWPYLMHERPAYLYSLEVQLTAQTSLGPVSDFYTLPVGIRTVAVTKSQFLINGKPFYFHGVNKHEDADIRGKGFDWPLLVKDFNLLRWLGANAFRTSHYPYAEEVMQMCDRYGIVVIDECPGVGLALPQFFNNVSLHHHMQVMEEVVRRDKNHPAVVMWSVANEPASHLESAGYYLKMVIAHTKSLDPSRPVTFVSNSNYAADKGAPYVDVICLNSYYSWYHDYGHLELIQLQLATQFENWYKKYQKPIIQSEYGAETIAGFHQDPPLMFTEEYQKSLLEQYHLGLDQKRRKYVVGELIWNFADFMTEQSPTRVLGNKKGIFTRQRQPKSAAFLLRERYWKIANETRYPHSVAKSQCLENSLFT

SEQ ID NO: 20 - protein, 313 aa, HOMO SAPIENS
AELCVNSLEKFHFKSWMSKHRKTYSTEEYHHRLQTFASNWRKINAHNNGNHTFKMALNQFSDMSFAEIKHKYLWSEPQNCSATKSNYLRGTGPYPPSVDWRKKGNFVSPVKNQGACGSCWTFSTTGALESAIAIATGKMLSLAEQQLVDCAQDFNNHGCQGGLPSQAFEYILYNKGIMGEDTYPYQGKDGYCKFQPGKAIGFVKDVANITIYDEEAMVEAVALYNPVSFAFEVTQDFMMYRTGIYSSTSCHKTPDKVNHAVLAVGYGEKNGIPYWIVKNSWGPQWGMNGYFLIERGKNMCGLAACASYPIPLV

SEQ ID NO: 21 - protein, 134 aa, HOMO SAPIENS
KPPQFTWAQWFETQHINMTSQQCTNAMQVINNYQRRCKNQNTFLLTTFANVVNVCGNPNMTCPSNKTRKNCHHSGSQVPLIHCNLTTPSPQNISNCRYAQTPANMFYIVACDNRDQRRDPPQYPVVPVHLDRII

SEQ ID NO: 22 - protein, 925 aa, HOMO SAPIENS
GHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 23 - protein, 452 aa, HOMO SAPIENS
APDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 24 - protein, 224 aa, HOMO SAPIENS
SPLQALDFFGNGPPVNYKTGNLYLRGPLKKSNAPLVNVTLYYEALCGGCRAFLIRELFPTWLLVMEILNVTLVPYGNAQEQNVSGRWEFKCQHGEEECKFNKVEACVLDELDMELAFLTIVCMEEFEDMERSLPLCLQLYAPGLSPDTIMECAMGDRGMQLMHANAQRTDALQPPHEYVPWVTVNGKPLEDQTQLLTLVCQLYQGKKPDVCPSSTSSLRSVCFK

SEQ ID NO: 25 - protein, 304 aa, HOMO SAPIENS
ATPALRFVAVGDWGGVPNAPFHTAREMANAKEIARTVQILGADFILSLGDNFYFTGVQDINDKRFQETFEDVFSDRSLRKVPWYVLAGNHDHLGNVSAQIAYSKISKRWNFPSPFYRLHFKIPQTNVSVAIFMLDTVTLCGNSDDFLSQQPERPRDVKLARTQLSWLKKQLAAAREDYVLVAGHYPVWSIAEHGPTHCLVKQLRPLLATYGVTAYLCGHDHNLQYLQDENGVGYVLSGAGNFMDPSKRHQRKVPNGYLRFHYGTEDSLGGFAYVEISSKEMTVTYIEASGKSLFKTRLPRRARP

SEQ ID NO: 26 - protein, 489 aa, HOMO SAPIENS
RPPNIVLIFADDLGYGDLGCYGHPSSTTPNLDQLAAGGLRFTDFYVPVSLCTPSRAALLTGRLPVRMGMYPGVLVPSSRGGLPLEEVTVAEVLAARGYLTGMAGKWHLGVGPEGAFLPPHQGFHRFLGIPYSHDQGPCQNLTCFPPATPCDGGCDQGLVPIPLLANLSVEAQPPWLPGLEARYMAFAHDLMADAQRQDRPFFLYYASHHTHYPQFSGQSFAERSGRGPFGDSLMELDAAVGTLMTAIGDLGLLEETLVIFTADNGPETMRMSRGGCSGLLRCGKGTTYEGGVREPALAFWPGHIAPGVTHELASSLDLLPTLAALAGAPLPNVTLDGFDLSPLLLGTGKSPRQSLFFYPSYPDEVRGVFAVRTGKYKAHFFTQGSAHSDTTADPACHASSSLTAHEPPLLYDLSKDPGENYNLLGGVAGATPEVLQALKQLQLLKAQLDAAVTFGPSQVARGEDPALQICCHPGCTPRPACCHCPDPHA

SEQ ID NO: 27 - protein, 354 aa, HOMO SAPIENS
KELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 28 - protein, 516 aa, HOMO SAPIENS
VFGVAAGTRRPNVVLLLTDDQDEVLGGMTPLKKTKALIGEMGMTFSSAYVPSALCCPSRASILTGKYPHNHHVVNNTLEGNCSSKSWQKIQEPNTFPAILRSMCGYQTFFAGKYLNEYGAPDAGGLEHVPLGWSYWYALEKNSKYYNYTLSINGKARKHGENYSVDYLTDVLANVSLDFLDYKSNFEPFFMMIATPAPHSPWTAAPQYQKAFQNVFAPRNKNFNIHGTNKHWLIRQAKTPMTNSSIQFLDNAFRKRWQTLLSVDDLVEKLVKRLEFTGELNNTYIFYTSDNGYHTGQFSLPIDKRQLYEFDIKVPLLVRGPGIKPNQTSKMLVANIDLGPTILDIAGYDLNKTQMDGMSLLPILRGASNLTWRSDVLVEYQGEGRNVTDPTCPSLSPGVSQCFPDCVCEDAYNNTYACVRTMSALWNLQYCEFDDQEVFVEVYNLTADPDQITNIAKTIDPELLGKMNYRLMMLQSCSGPTCRTPGVFDPGYRFDPRLMFSNRGSVRTRRFSKHLL

SEQ ID NO: 29 - protein, 497 aa, HOMO SAPIENS
SGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 30 - protein, 654 aa, HOMO SAPIENS
LRNATQRMFEIDYSRDSFLKDGQPFRYISGSIHYSRVPRFYWKDRLLKMKMAGLNAIQTYVPWNFHEPWPGQYQFSEDHDVEYFLRLAHELGLLVILRPGPYICAEWEMGGLPAWLLEKESILLRSSDPDYLAAVDKWLGVLLPKMKPLLYQNGGPVITVQVENEYGSYFACDFDYLRFLQKRFRHHLGDDVVLFTTDGAHKTFLKCGALQGLYTTVDFGTGSNITDAFLSQRKCEPKGPLINSEFYTGWLDHWGQPHSTIKTEAVASSLYDILARGASVNLYMFIGGTNFAYWNGANSPYAAQPTSYDYDAPLSEAGDLTEKYFALRNIIQKFEKVPEGPIPPSTPKFAYGKVTLEKLKTVGAALDILCPSGPIKSLYPLTFIQVKQHYGFVLYRTTLPQDCSNPAPLSSPLNGVHDRAYVAVDGIPQGVLERNNVITLNITGKAGATLDLLVENMGRVNYGAYINDFKGLVSNLTLSSNILTDWTIFPLDTEDAVRSHLGGWGHRDSGHHDEAWAHNSSNYTLPAFYMGNFSIPSGIPDLPQDTFIQFPGWTKGQVWINGFNLGRYWPARGPQLTLFVPQHILMTSAPNTITVLELEWAPCSSDDPELCAVTFVDRPVIGSSVTYDHPSKPVEKRLMPPPPQKNKDSWLDHV

SEQ ID NO: 31 - protein, 394 aa, HOMO SAPIENS
LDNGLLQTPPMGWLAWERFRCNINCDEDPKNCISEQLFMEMADRMAQDGWRDMGYTYLNIDDCWIGGRDASGRLMPDPKRFPHGIPFLADYVHSLGLKLGIYADMGNFTCMGYPGTTLDKVVQDAQTFAEWKVDMLKLDGCFSTPEERAQGYPKMAAALNATGRPIAFSCSWPAYEGGLPPRVNYSLLADICNLWRNYDDIQDSWWSVLSILNWFVEHQDILQPVAGPGHWNDPDMLLIGNFGLSLEQSRAQMALWTVLAAPLLMSTDLRTISAQNMDILQNPLMIKINQDPLGIQGRRIHKEKSLIEVYMRPLSNKASALVFFSCRTDMPYRYHSSLGQLNFTGSVIYEAQDVYSGDIISGLRDETNFTVIINPSGVVMWYLYPIKNLEMSQQ

SEQ ID NO: 32 - protein, 583 aa, HOMO SAPIENS
LSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 33 - protein, 170 aa, HOMO SAPIENS
HLKKPSQLSSFSWDNCDEGKDPAVIRSLTLEPDPIIVPGNVTLSVMGSTSVPLSSPLKVDLVLEKEVAGLWIKIPCTDYIGSCTFEHFCDVLDMLIPTGEPCPEPLRTYGLPCHCPFKEGTYSLPKSEFVVPDLELPSWLTTGNYRIESVLSSSGKRLGCIKIAASLKGI

SEQ ID NO: 34 - protein, 323 aa, HOMO SAPIENS
SSPLPLVVNTWPFKNATEAAWRALASGGSALDAVESGCAMCEREQCDGSVGFGGSPDELGETTLDAMIMDGTTMDVGAVGDLRRIKNAIGVARKVLEHTTHTLLVGESATTFAQSMGFINEDLSTTASQALHSDWLARNCQPNYWRNVIPDPSKYCGPYKPPGILKQDIPIHKETEDDRGHDTIGMVVIHKTGHIAAGTSTNGIKFKIHGRVGDSPIPGAGAYADDTAGAAAATGNGDILMRFLPSYQAVEYMRRGEDPTIACQKVISRIQKHFPEFFGAVICANVTGSYGAACNKLSTFTQFSFMVYNSEKNQPTEEKVDCI

SEQ ID NO: 35 - protein, 525 aa, HOMO SAPIENS
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 36 - protein, 315 aa, HOMO SAPIENS
QLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANKGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 37 - protein, 496 aa, HOMO SAPIENS
APQPPNILLLLMDDMGWGDLGVYGEPSRETPNLDRMAAEGLLFPNFYSANPLCSPSRAALLTGRLPIRNGFYTTNAHARNAYTPQEIVGGIPDSEQLLPELLKKAGYVSKIVGKWHLGHRPQFHPLKHGFDEWFGSPNCHFGPYDNKARPNIPVYRDWEMVGRYYEEFPINLKTGEANLTQIYLQEALDFIKRQARHHPFFLYWAVDATHAPVYASKPFLGTSQRGRYGDAVREIDDSIGKILELLQDLHVADNTFVFFTSDNGAALISAPEQGGSNGPFLCGKQTTFEGGMREPALAWWPGHVTAGQVSHQLGSIMDLFTTSLALAGLTPPSDRAIDGLNLLPTLLQGRLMDRPIFYYRGDTLMAATLGQHKAHFWTWTNSWENFRQGIDFCPGQNVSGVTTHNLEDHTKLPLIFHLGRDPGERFPLSFASAEYQEALSRITSVVQQHQEALVPAQPQLNVCNWAVMNWAPPGCEKLGKCLTPPESIPKKCLWSH

SEQ ID NO: 38 - protein, 626 aa, HOMO SAPIENS
APHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 39 - protein, 376 aa, HOMO SAPIENS
GKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 40 - protein, 475 aa, HOMO SAPIENS
LRPALRALGSLHLPTNPTSLPAVAKNYSVLYFQQKVDHFGFNTVKTFNQRYLVADKYWKKNGGSILFYTGNEGDIIWFCNNTGFMWDVAEELKAMLVFAEHRYYGESLPFGDNSFKDSRHLNFLTSEQALADFAELIKHLKRTIPGAENQPVIAIGGSYGGMLAAWFRMKYPHMVVGALAASAPIWQFEDLVPCGVFMKIVTTDFRKSGPHCSESIHRSWDAINRLSNTGSGLQWLTGALHLCSPLTSQDIQHLKDWISETWVNLAMVDYPYASNFLQPLPAWPIKVVCQYLKNPNVSDSLLLQNIFQALNVYYNYSGQVKCLNISETATSSLGTLGWSYQACTEVVMPFCTNGVDDMFEPHSWNLKELSDDCFQQWGVRPRPSWITTMYGGKNISSHTNIVFSNGELDPWSGGGVTKDITDTLVAVTISEGAHHLDLRTKNALDPMSVLLARSLEVRHMKNWIRDFYDSAGKQH

SEQ ID NO: 41 - protein, 298 aa, HOMO SAPIENS
DSRAPFTPTWPRSREREAAAFRESLNRHRYLNSLFPSENSTAFYGINQFSYLFPEEFKAIYLRSKPSKFPRYSAEVHMSIPNVSLPLRFDWRDKQVVTQVRNQQMCGGCWAFSVVGAVESAYAIKGKPLEDLSVQQVIDCSYNNYGCNGGSTLNALNWLNKMQVKLVKDSEYPFKAQNGLCHYFSGSHSGFSIKGYSAYDFSDQEDEMAKALLTFGPLVVIVDAVSWQDYLGGIIQHHCSSGEANHAVLITGFDKTGSTPYWIVRNSWGSSWGVDGYAHVKMGSNVCGIADSVSSIFV

SEQ ID NO: 42 - protein, 314 aa, HOMO SAPIENS
LYPEEILDTHWELWKKTHRKQYNNKVDEISRRLIWEKNLKYISIHNLEASLGVHTYELAMNHLGDMTSEEVVQKMTGLKVPLSHSRSNDTLYIPEWEGRAPDSVDYRKKGYVTPVKNQGQCGSCWAFSSVGALEGQLKKKTGKLLNLSPQNLVDCVSENDGCGGGYMTNAFQYVQKNRGIDSEDAYPYVGQEESCMYNPTGKAAKCRGYREIPEGNEKALKRAVARVGPVSVAIDASLTSFQFYSKGVYYDESCNSDNLNHAVLAVGYGIQKGNKHWIIKNSWGENWGNKGYILMARNKNNACGIANLASFPKM

SEQ ID NO: 43 - protein, 279 aa, HOMO SAPIENS
DPPAPLPLVIWHGMGDSCCNPLSMGAIKKMVEKKIPGIYVLSLEIGKTLMEDVENSFFLNVNSQVTTVCQALAKDPKLQQGYNAMGFSQGGQFLRAVAQRCPSPPMINLISVGGQHQGVFGLPRCPGESSHICDFIRKTLNAGAYSKVVQERLVQAEYWHDPIKEDVYRNHSIFLADINQERGINESYKKNLMALKKFVMVKFLNDSIVDPVDSEWFGFYRSGQAKETIPLQETSLYTQDRLGLKEMDNAGQLVFLATEGDHLQLSEEWFYAHIIPFLG

SEQ ID NO: 44 - protein, 482 aa, HOMO SAPIENS
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 45 - protein, 560 aa, HOMO SAPIENS
KTANAFKPNILLIMADDLGTGDLGCYGNNTLRTPNIDQLAEEGVRLTQHLAAAPLCTPSRAAFLTGRHSFRSGMDASNGYRALQWNAGSGGLPENETTFARILQQHGYATGLIGKWHQGVNCASRGDHCHHPLNHGFDYFYGMPFTLTNDCDPGRPPEVDAALRAQLWGYTQFLALGILTLAAGQTCGFFSVSARAVTGMAGVGCLFFISWYSSFGFVRRWNCILMRNHDVTEQPMVLEKTASLMLKEAVSYIERHKHGPFLLFLSLLHVHIPLVTTSAFLGKSQHGLYGDNVEEMDWLIGKVLNAIEDNGLKNSTFTYFTSDHGGHLEARDGHSQLGGWNGIYKGGKGMGGWEGGIRVPGIFHWPGVLPAGRVIGEPTSLMDVFPTVVQLVGGEVPQDRVIDGHSLVPLLQGAEARSAHEFLFHYCGQHLHAARWHQKDSGSVWKVHYTTPQFHPEGAGACYGRGVCPCSGEGVTHHRPPLLFDLSRDPSEARPLTPDSEPLYHAVIARVGAAVSEHRQTLSPVPQQFSMSNILWKPWLQPCCGHFPFCSCHEDGDGTP

SEQ ID NO: 46 - protein, 439 aa, HOMO SAPIENS
DTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNIAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 47 - protein, 720 aa, HOMO SAPIENS
DEAREAAAVRALVARLLGPGPAADFSVSVERALAAKPGLDTYSLGGGGAARVRVRGSTGVAAAAGLHRYLRDFCGCHVAWSGSQLRLPRPLPAVPGELTEATPNRYRYYQNVCTQSYSFVWWDWARWEREIDWMALNGINLALAWSGQEAIWQRVYLALGLTQAEINEFFTGPAFLAWGRMGNLHTWDGPLPPSWHIKQLYLQHRVLDQMRSFGMTPVLPAFAGHVPEAVTRVFPQVNVTKMGSWGHFNCSYSCSFLLAPEDPIFPIIGSLFLRELIKEFGTDHIYGADTFNEMQPPSSEPSYLAAATTAVYEAMTAVDTEAVWLLQGWLFQHQPQFWGPAQIRAVLGAVPRGRLLVLDLFAESQPVYTRTASFQGQPFIWCMLHNFGGNHGLFGALEAVNGGPEAARLFPNSTMVGTGMAPEGISQNEVVYSLMAELGWRKDPVPDLAAWVTSFAARRYGVSHPDAGAAWRLLLRSVYNCSGEACRGHNRSPLVRRPSLQMNTSIWYNRSDVFEAWRLLLTSAPSLATSPAFRYDLLDLTRQAVQELVSLYYEEARSAYLSKELASLLRAGGVLAYELLPALDEVLASDSRFLLGSWLEQARAAAVSEAEADFYEQNSRYQLTLWGPEGNILDYANKQLAGLVANYYTPRWRLFLEALVDSVAQGIPFQQHQFDKNVFQLEQAFVLSKQRYPSQPRGDTVDLAKKIFLKYYPRWVAGSW

SEQ ID NO: 48 - protein, 643 aa, HOMO SAPIENS
YVLDDSDGLGREFDGIGAVSGGGATSRLLVNYPEPYRSQILDYLFKPNFGASLHILKVEIGGDGQTTDGTEPSHMHYALDENYFRGYEWWLMKEAKKRNPNITLIGLPWSFPGWLGKGFDWPYVNLQLTAYYVVTWIVGAKRYHDLDIDYIGIWNERSYNANYIKILRKMLNYQGLQRVKIIASDNLWESISASMLLDAELFKVVDVIGAHYPGTHSAKDAKLTGKKLWSSEDFSTLNSDMGAGCWGRILNQNYINGYMTSTIAWNLVASYYEQLPYGRCGLMTAQEPWSGHYVVESPVWVSAHTTQFTQPGWYYLKTVGHLEKGGSYVALTDGLGNLTIIIETMSHKHSKCIRPFLPYFNVSQQFATFVLKGSFSEIPELQVWYTKLGKTSERFLFKQLDSLWLLDSDGSFTLSLHEDELFTLTTLTTGRKGSYPLPPKSQPFPSTYKDDFNVDYPFFSEAPNFADQTGVFEYFTNIEDPGEHHFTLRQVLNQRPITWAADASNTISIIGDYNWTNLTIKCDVYIETPDTGGVFIAGRVNKGGILIRSARGIFFWIFANGSYRVTGDLAGWIIYALGRVEVTAKKWYTLTLTIKGHFASGMLNDKSLWTDIPVNFPKNGWAAIGTHSFEFAQFDNFLVEATR

SEQ ID NO: 49 - protein, 132 aa, HOMO SAPIENS
EPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 50 - protein, 347 aa, HOMO SAPIENS
TDCPCPEPELCRPIRHHPDFEVFVFDVGQKTWKSYDWSQITTVATFGKYDSELMCYAHSKGARVVLKGDVSLKDIIDPAFRASWIAQKLNLAKTQYMDGINIDIEQEVNCLSPEYDALTALVKETTDSFHREIEGSQVTFDVAWSPKNIDRRCYNYTGIADACDFLFVMSYDEQSQIWSECIAAANAPYNQTLTGYNDYIKMSINPKKLVMGVPWYGYDYTCLNLSEDHVCTIAKVPFRGAPCSDAAGRQVPYKTIMKQINSSISGNLWDKDQRAPYYNYKDPAGHFHQVWYDNPQSISLKATYIQNYRLRGIGMWNANCLDYSGDAVAKQQTEEMWEVLKPKLLQR

SEQ ID NO: 51 - protein, 331 aa, HOMO SAPIENS
SPPAAPRFNVSLDSVPELRWLPVLRHYDLDLVRAAMAQVIGDRVPKWVHVLIGKVVLELERFLPQPFTGEIRGMCDFMNLSLADCLLVNLAYESSVFCTSIVAQDSRGHIYHGRNLDYPFGNVLRKLTVDVQFLKNGQIAFTGTTFIGYVGLWTGQSPHKFTVSGDERDKGWWWENAIAALFRRHIPVSWLIRATLSESENFEAAVGKLAKTPLIADVYYIVGGTSPREGVVITRNRDGPADIWPLDPLNGAWFRVETNYDHWKPAPKEDDRRTSAIKALNATGQANLSLEALFQILSVVPVYNNFTIYTTVMSAGSPDKYMTRIRNPSRK

SEQ ID NO: 52 - protein, 414 aa, HOMO SAPIENS
FRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 53 - protein, 445 aa, HOMO SAPIENS
AKLVCYFTNWAQYRQGEARFLPKDLDPSLCTHLIYAFAGMTNHQLSTTEWNDETLYQEFNGLKKMNPKLKTLLAIGGWNFGTQKFTDMVATANNRQTFVNSAIRFLRKYSFDGLDLDWEYPGSQGSPAVDKERFTTLVQDLANAFQQEAQTSGKERLLLSAAVPAGQTYVDAGYEVDKIAQNLDFVNLMAYDFHGSWEKVTGHNSPLYKRQEESGAAASLNVDAAVQQWLQKGTPASKLILGMPTYGRSFTLASSSDTRVGAPATGSGTPGPFTKEGGMLAYYEVCSWKGATKQRIQDQKVPYIFRDNQWVGFDDVESFKTKVSYLKQKGLGGAMVWALDLDDFAGFSCNQGRYPLIQTLRQELSLPYLPSGTPELEVPKPGQPSEPEHGPSPGQDTFCQGKADGLYPNPRERSSFYSCAAGRLFQQSCPTGLVFSNSCKCCTWN

SEQ ID NO: 54 - protein, 374 aa, HOMO SAPIENS
QHAPPWTEDCRKSTYPPSGPTYRGAVPWYTINLDLPPYKRWHELMLDKAPVLKVIVNSLKNMINTFVPSGKIMQVVDEKLPGLLGNFPGPFEEEMKGIAAVTDIPLGEIISFNIFYELFTICTSIVAEDKKGHLIHGRNMDFGVFLGWNINNDTWVITEQLKPLTVNLDFQRNNKTVFKASSFAGYVGMLTGFKPGLFSLTLNERFSINGGYLGILEWILGKKDVMWIGFLTRTVLENSTSYEEAKNLLTKTKILAPAYFILGGNQSGEGCVITRDRKESLDVYELDAKQGRWYVVQTNYDRWKHPFFLDDRRTPAKMCLNRTSQENISFETMYDVLSTKPVLNKLTVYTTLIDVTKGQFETYLRDCPDPCIGW

SEQ ID NO: 55 - protein, 515 aa, HOMO SAPIENS
GVYYATAYWMPAEKTVQVKNVMDKNGDAYGFYNNSVKTTGWGILEIRAGYGSQTLSNEIIMFVAGFLEGYLTAPHMNDHYTNLYPQLITKPSIMDKVQDFMEKQDKWTRKNIKEYKTDSFWRHTGYVMAQIDGLYVGAKKRAILEGTKPMTLFQIQFLNSVGDLLDLIPSLSPTKNGSLKVFKRWDMGHCSALIKVLPGFENILFAHSSWYTYAAMLRIYKHWDFNVIDKDTSSSRLSFSSYPGFLESLDDFYILSSGLILLQTTNSVFNKTLLKQVIPETLLSWQRVRVANMMADSGKRWADIFSKYNSGTYNNQYMVLDLKKVKLNHSLDKGTLYIVEQIPTYVEYSEQTDVLRKGYWPSYNVPFHEKIYNWSGYPLLVQKLGLDYSYDLAPRAKIFRRDQGKVTDTASMKYIMRYNNYKKDPYSRGDPCNTICCREDLNSPNPSPGGCYDTKVADIYLASQYTSYAISGPTVQGGLPVFRWDRFNKTLHQGMPEVYNFDFITMKPILKLDIK

SEQ ID NO: 56 - protein, 662 aa, HOMO SAPIENS
QEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAVARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEGAVTAVAICCRSRHLAQASQELQ

SEQ ID NO: 57 - protein, 379 aa, HOMO SAPIENS
AGRHPPVVLVPGDLGNQLEAKLDKPTVVHYLCSKKTESYFTIWLNLELLLPVIIDCWIDNIRLVYNKTSRATQFPDGVDVRVPGFGKTFSLEFLDPSKSSVGSYFHTMVESLVGWGYTRGEDVRGAPYDWRRAPNENGPYFLALREMIEEMYQLYGGPVVLVAHSMGNMYTLYFLQRQPQAWKDKYIRAFVSLGAPWGGVAKTLRVLASGDNNRIPVIGPLKIREQQRSAVSTSWLLPYNYTWSPEKVFVQTPTINYTLRDYRKFFQDIGFEDGWLMRQDTEGLVEATMPPGVQLHCLYGTGVPTPDSFYYESFPDRDPKICFGDGDGTVNLKSALQCQAWQSRQEHQVLLQELPGSEHIEMLANATTLAYLKRVLLGP

SEQ ID NO: 58 - protein, 548 aa, HOMO SAPIENS
IPAPGGRWARDGQVPPASRSRSVLLDVSAGQLLMVDGRHPDAVAWANLTNAIRETGWAFLELGTSGQYNDSLQAYAAGVVEAAVSEELIYMHWMNTVVNYCGPFEYEVGYCERLKSFLEANLEWMQEEMESNPDSPYWHQVRLTLLQLKGLEDSYEGRVSFPAGKFTIKPLGFLLLQLSGDLEDLELALNKTKIKPSLGSGSCSALIKLLPGQSDLLVAHNTWNNYQHMLRVIKKYWLQFREGPWGDYPLVPGNKLVFSSYPGTIFSCDDFYILGSGLVTLETTIGNKNPALWKYVRPRGCVLEWVRNIVANRLASDGATWADIFKRFNSGTYNNQWMIVDYKAFIPGGPSPGSRVLTILEQIPGMVVVADKTSELYQKTYWASYNIPSFETVFNASGLQALVAQYGDWFSYDGSPRAQIFRRNQSLVQDMDSMVRLMRYNDFLHDPLSLCKACNPQPNGENAISARSDLNPANGSYPFQALRQRSHGGIDVKVTSMSLARILSLLAASGPTWDQVPPFQWSTSPFSGLLHMGQPDLWKFAPVKVSWD

SEQ ID NO: 59 - protein, 334 aa, HOMO SAPIENS
ATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 60 - protein, 294 aa, HOMO SAPIENS
RPHGDTAKKPIIGILMQKCRNKVMKNYGRYYIAASYVKYLESAGARVVPVRLDLTEKDYEILFKSINGILFPGGSVDLRRSDYAKVAKIFYNLSIQSFDDGDYFPVWGTCLGFEELSLLISGECLLTATDTVDVAMPLNFTGGQLHSRMFQNFPTELLLSLAVEPLTANFHKWSLSVKNFTMNEKLKKFFNVLTTNTDGKIEFISTMEGYKYPVYGVQWHPEKAPYEWKNLDGISHAPNAVKTAFYLAEFFVNEARKNNHHFKSESEEEKALIYQFSPIYTGNISSFQQCYIFD

SEQ ID NO: 61 - protein, 509 aa, HOMO SAPIENS
GFLYPLVDFCISGKTRGQKPNFVIILADDMGWGDLGANWAETKDTANLDKMASEGMRFVDFHAAASTCSPSRASLLTGRLGLRNGVTRNFAVTSVGGLPLNETTLAEVLQQAGYVTGIIGKWHLGHHGSYHPNFRGFDYYFGIPYSHDMGCTDTPGYNHPPCPACPQGDGPSRNLQRDCYTDVALPLYENLNIVEQPVNLSSLAQKYAEKATQFIQRASTSGRPFLLYVALAHMHVPLPVTQLPAAPRGRSLYGAGLWEMDSLVGQIKDKVDHTVKENTFLWFTGDNGPWAQKCELAGSVGPFTGFWQTRQGGSPAKQTTWEGGHRVPALAYWPGRVPVNVTSTALLSVLDIFPTVVALAQASLPQGRRFDGVDVSEVLFGRSQPGHRVLFHPNSGAAGEFGALQTVRLERYKAFYITGGARACDGSTGPELQHKFPLIFNLEDDTAEAVPLERGGAEYQAVLPEVRKVLADVLQDIANDNISSADYTQDPSVTPCCNPYQIACRCQAA

SEQ ID NO: 62 - protein, 546 aa, HOMO SAPIENS
QDWKAERSQDPFEKCMQDPDYEQLLKVVTWGLNRTLKPQRVIVVGAGVAGLVAAKVLSDAGHKVTILEADNRIGGRIFTYRDQNTGWIGELGAMRMPSSHRILHKLCQGLGLNLTKFTQYDKNTWTEVHEVKLRNYVVEKVPEKLGYALRPQEKGHSPEDIYQMALNQALKDLKALGCRKAMKKFERHTLLEYLLGEGNLSRPAVQLLGDVMSEDGFFYLSFAEALRAHSCLSDRLQYSRIVGGWDLLPRALLSSLSGLVLLNAPVVAMTQGPHDVHVQIETSPPARNLKVLKADVVLLTASGPAVKRITFSPPLPRHMQEALRRLHYVPATKVFLSFRRPFWREEHIEGGHSNTDRPSRMIFYPPPREGALLLASYTWSDAAAAFAGLSREEALRLALDDVAALHGPVVRQLWDGTGVVKRWAEDQHSQGGFVVQPPALWQTEKDDWTVPYGRIYFAGEHTAYPHGWVETAVKSALRAAIKINSRKGPASDTASPEGHASDMEGQGHVHGVASSPSHDLAKEEGSHPPVQGQLSLQNTTHTRTSH

SEQ ID NO: 63 - protein, 368 aa, HOMO SAPIENS
ENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 64 - protein, 416 aa, HOMO SAPIENS
VPIDDPEDGGKHWVVIVAGSNGWYNYRHQADACHAYQIIHRNGIPDEQIVVMMYDDIAYSEDNPTPGIVINRPNGTDVYQGVPKDYTGEDVTPQNFLAVLRGDAEAVKGIGSGKVLKSGPQDHVFIYFTDHGSTGILVFPNEDLHVKDLNETIHYMYKHKMYRKMVFYIEACESGSMMNHLPDNINVYATTAANPRESSYACYYDEKRSTYLGDWYSVNWMEDSDVEDLTKETLHKQYHLVKSHTNTSHVMQYGNKTISTMKVMQFQGMKRKASSPVPLPPVTHLDLTPSPDVPLTIMKRKLMNTNDLEESRQLTEEIQRHLDARHLIEKSVRKIVSLLAASEAEVEQLLSERAPLTGHSCYPEALLHFRTHCFNWHSPTYEYALRHLYVLVNLCEKPYPLHRIKLSMDHVCLGHY

SEQ ID NO: 65 - protein, 500 aa, HOMO SAPIENS
IGFRFASYINNDMVLQKEPAGAVIWGFGTPGATVTVTLRQGQETIMKKVTSVKAHSDTWMVVLDPMKPGGPFEVMAQQTLEKINFTLRVHDVLFGDVWLCSGQSNMQMTVLQIFNATRELSNTAAYQSVRILSVSPIQAEQELEDLVAVDLQWSKPTSENLGHGYFKYMSAVCWLFGRHLYDTLQYPIGLIASSWGGTPIEAWSSGRSLKACGVPKQGSIPYDSVTGPSKHSVLWNAMIHPLCNMTLKGVVWYQGESNINYNTDLYNCTFPALIEDWRETFHRGSQGQTERFFPFGLVQLSSDLSKKSSDDGFPQIRWHQTADFGYVPNPKMPNTFMAVAMDLCDRDSPFGSIHPRDKQTVAYRLHLGARALAYGEKNLTFEGPLPEKIELLAHKGLLNLTYYQQIQVQKKDNKIFEISCCSDHRCKWLPASMNTVSTQSLTLAIDSCHGTVVALRYAWTTWPCEYKQCPLYHPSSALPAPPFIAFITDQGPGHQSNVAK

SEQ ID NO: 66 - protein, 490 aa, HOMO SAPIENS
SLLRRLGEHIQQFQESSAQGLGLSLGPGAAALPKVGWLEQLLDPFNVSDRRSFLQRYWVNDQHWVGQDGPIFLHLGGEGSLGPGSVMRGHPAALAPAWGALVISLEHRFYGLSIPAGGLEMAQLRFLSSRLALADVVSARLALSRLFNISSSSPWICFGGSYAGSLAAWARLKFPHLIFASVASSAPVRAVLDFSEYNDVVSRSLMSTAIGGSLECRAAVSVAFAEVERRLRSGGAAQAALRTELSACGPLGRAENQAELLGALQALVGGVVQYDGQTGAPLSVRQLCGLLLGGGGNRSHSTPYCGLRRAVQIVLHSLGQKCLSFSRAETVAQLRSTEPQLSGVGDRQWLYQTCTEFGFYVTCENPRCPFSQLPALPSQLDLCEQVFGLSALSVAQAVAQTNSYYGGQTPGANKVLFVNGDTDPWHVLSVTQALGSSESTLLIRTGSHCLDMAPERPSDSPSLRLGRQNIFQQLQTWLKLAKESQIKGEV

SEQ ID NO: 67 - protein, 280 aa, HOMO SAPIENS
GLYFRRGQTCYRPLRGDGLAPLGRSTYPRPHEYLSPADLPKSWDWRNVDGVNYASITRNQHIPQYCGSCWAHASTSAMADRINIKRKGAWPSTLLSVQNVIDCGNAGSCEGGNDLSVWDYAHQHGIPDETCNNYQAKDQECDKFNQCGTCNEFKECHAIRNYTLWRVGDYGSLSGREKMMAEIYANGPISCGIMATERLANYTGGIYAEYQDTTYINHVVSVAGWGISDGTEYWIVRNSWGEPWGERGWLRIVTSTYKDGKGARYNLAIEEHCTFGDPIV

SEQ ID NO: 68 - protein, 465 aa, HOMO SAPIENS
APAQPRAASFQAWGPPSPELLAPTRFALEMFNRGRAAGTRAVLGLVRGRVRRAGQGSLYSLEATLEEPPCNDPMVCRLPVSKKTLLCSFQVLDELGRHVLLRKDCGPVDTKVPGAGEPKSAFTQGSAMISSLSQNHPDNRNETFSSVISLLNEDPLSQDLPVKMASIFKNFVITYNRTYESKEEARWRLSVFVNNMVRAQKIQALDRGTAQYGVTKFSDLTEEEFRTIYLNTLLRKEPGNKMKQAKSVGDLAPPEWDWRSKGAVTKVKDQGMCGSCWAFSVTGNVEGQWFLNQGTLLSLSEQELLDCDKMDKACMGGLPSNAYSAIKNLGGLETEDDYSYQGHMQSCNFSAEKAKVYINDSVELSQNEQKLAAWLAKRGPISVAINAFGMQFYRHGISRPLRPLCSPWLIDHAVLLVGYGNRSDVPFWAIKNSWGTDWGEKGYYYLHRGSGACGVNTMASSAVVD

SEQ ID NO: 69 - protein, 478 aa, HOMO SAPIENS
AELRAPPDKIAIIGAGIGGTSAAYYLRQKFGKDVKIDLFEREEVGGRLATMMVQGQEYEAGGSVIHPLNLHMKRFVKDLGLSAVQASGGLLGIYNGETLVFEESNWFIINVIKLVWRYGFQSLRMHMWVEDVLDKFMRIYRYQSHDYAFSSVEKLLHALGGDDFLGMLNRTLLETLQKAGFSEKFLNEMIAPVMRVNYGQSTDINAFVGAVSLSCSDSGLWAVEGGNKLVCSGLLQASKSNLISGSVMYIEEKTKTKYTGNPTKMYEVVYQIGTETRSDFYDIVLVATPLNRKMSNITFLNFDPPIEEFHQYYQHIVTTLVKGELNTSIFSSRPIDKFGLNTVLTTDNSDLFINSIGIVPSVREKEDPEPSTDGTYVWKIFSQETLTKAQILKLFLSYDYAVKKPWLAYPHYKPPEKCPSIILHDRLYYLNGIECAASAMEMSAIAAHNAALLAYHRWNGHTDMIDQDGLYEKLKTEL

SEQ ID NO: 70 - protein, 471 aa, HOMO SAPIENS
GARRAPDPGFQERFFQQRLDHFNFERFGNKTFPQRFLVSDRFWVRGEGPIFFYTGNEGDVWAFANNSAFVAELAAERGALLVFAEHRYYGKSLPFGAQSTQRGHTELLTVEQALADFAELLRALRRDLGAQDAPAIAFGGSYGGMLSAYLRMKYPHLVAGALAASAPVLAVAGLGDSNQFFRDVTADFEGQSPKCTQGVREAFRQIKDLFLQGAYDTVRWEFGTCQPLSDEKDLTQLFMFARNAFTVLAMMDYPYPTDFLGPLPANPVKVGCDRLLSEAQRITGLRALAGLVYNASGSEHCYDIYRLYHSCADPTGCGTGPDARAWDYQACTEINLTFASNNVTDMFPDLPFTDELRQRYCLDTWGVWPRPDWLLTSFWGGDLRAASNIIFSNGNLDPWAGGGIRRNLSASVIAVTIQGGAHHLDLRASHPEDPASVVEARKLEATIIGEWVKAARREQQPALRGGPRLSL

SEQ ID NO: 71 - protein, 275 aa, HOMO SAPIENS
APAPHRASYKPVIVVHGLFDSSYSFRHLLEYINETHPGTVVTVLDLFDGRESLRPLWEQVQGFREAVVPIMAKAPQGVHLICYSQGGLVCRALLSVMDDHNVDSFISLSSPQMGQYGDTDYLKWLFPTSMRSNLYRICYSPWGQEFSICNYWHDPHHDDLYLNASSFLALINGERDHPNATVWRKNFLRVGHLVLIGGPDDGVITPWQSSFFGFYDANETVLEMEEQLVYLRDSFGLKTLLARGAIVRCPMAGISHTAWHSNRTLYETCIEPWLS

SEQ ID NO: 72 - protein, 508 aa, HOMO SAPIENS
QDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDFIQLHKLLRKSTFKNAKLYGPDVGQPRRKTAKMLKSFLKAGGEVIDSVTWHHYYLNGRTATKEDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 73 - protein, 452 aa, HOMO SAPIENS
KAICKNGISKRTFEEIKEEIASCGDVAKAIINLAVYGKAQNRSYERLALLVDTVGPRLSGSKNLEKAIQIMYQNLQQDGLEKVHLEPVRIPHWERGEESAVMLEPRIHKIAILGLGSSIGTPPEGITAEVLVVTSFDELQRRASEARGKIVVYNQPYINYSRTVQYRTQGAVEAAKVGALASLIRSVASFSIYSPHTGIQEYQDGVPKIPTACITVEDAEMMSRMASHGIKIVIQLKMGAKTYPDTDSFNTVAEITGSKYPEQVVLVSGHLDSWDVGQGAMDDGGGAFISWEALSLIKDLGLRPKRTLRLVLWTAEEQGGVGAFQYYQLHKVNISNYSLVMESDAGTFLPTGLQFTGSEKARAIMEEVMSLLQPLNITQVLSHGEGTDINFWIQAGVPGASLLDDLYKYFFFHHSHGDTMTVMDPKQMNVAAAVWAVVSYVVADMEEMLPRS

SEQ ID NO: 74 - protein, 341 aa, HOMO SAPIENS
SQEAGTGAGAGSLAGSCGCGTPQRPGAHGSSAAAHRYSREANAPGPVPGERQLAHSKMVPIPAGVFTMGTDDPQIKQDGEAPARRVTIDAFYMDAYEVSNTEFEKFVNSTGYLTEAEKFGDSFVFEGMLSEQVKTNIQQAVAAAPWWLPVKGANWRHPEGPDSTILHRPDHPVLHVSWNDAVAYCTWAGKRLPTEAEWEYSCRGGLHNRLFPWGNKLQPKGQHYANIWQGEFPVTNTGEDGFQGTAPVDAFPPNGYGLYNIVGNAWEWTSDWWTVHHSVEETLNPKGPPSGKDRVKKGGSYMCHRSYCYRYRCAARSQNTPDSSASNLGFRCAADRLPTMD